(19) United States
(12) Patent Application Publication
(10) Pub. No.: US 2025/0134986 A1
(43) Pub. Date: May 1, 2025

(54) STABILIZED LIPID NANOPARTICLE MRNA COMPOSITIONS AND USES THEREOF

(71) Applicant: VERNDARI, INC., Sacramento, CA (US)

(72) Inventors: Azadeh KHEIROLOMOOM, Sacramento, CA (US); Thomas James ELLISON, Sacramento, CA (US); George Charles TALBOTT, Fair Oaks, CA (US); Daniel Robert HENDERSON, Napa, CA (US)

(21) Appl. No.: 18/692,309

(22) PCT Filed: Sep. 15, 2022
(86) PCT No.: PCT/US2022/043674

§ 371 Date: Mar. 14, 2024

Related U.S. Application Data
(60) Provisional application No. 63/244,717, filed on Sep. 15, 2021.
(60) Provisional application No. 63/275,395, filed on Nov. 3, 2021.
(60) Provisional application No. 63/284,614, filed on Nov. 30, 2021.

(51) Int. Cl. A61K 39/215; A61K 9/1271; A61K 9/51; A61K 39/00; A61K 47/10; A61K 47/26; A61K 47/36; A61M 37/00; C12N 7/00

(52) U.S. Cl. CPC A61K 39/215; A61K 9/1271; A61K 9/5123; A61K 47/10; A61K 47/26; A61K 47/36; A61M 37/0015; C12N 7/00; A61K 2039/53; A61K 2039/54; A61K 2039/55555; A61M 2037/0023; C12N 2770/20034

(57) ABSTRACT

Provided herein are, inter alia, compositions or formulations comprising a nucleic acid (e.g. a RNA molecule) encapsulated in a lipid nanoparticle (LNP) and their uses.

### CROSS-REFERENCE TO RELATED APPLICATIONS

This application claims the benefit of priority to U.S. Provisional Application 63/244,717 filed on Sep. 15, 2021, U.S. Provisional Application No. 63/275,395 filed on Nov. 3, 2021 and U.S. Provisional Application No. 63/284,614 filed on Nov. 30, 2021. The entire contents of these applications are incorporated herein by reference in their entirety.

### BACKGROUND

The outbreak of COVID-19 pandemic is caused by a highly pathogenic coronavirus severe acute respiratory syndrome coronavirus-2 (SARS-COV-2). mRNA LNP vaccines were the first to be granted FDA Emergency Use Authorization. This has changed vaccines globally. But mRNA vaccines are dependent on freezing temperatures for storage and distribution as well as complex preparation by medical personnel for administration. There is an urgent need to stabilize mRNA LNP vaccines and simplify vaccine administration for global distribution not only for SARS-COV-2 vaccines but for many vaccine and non-vaccine applications of mRNA LNPs.

### SUMMARY

Provided herein are, inter alia, compositions or formulations comprising a nucleic acid (e.g. a RNA molecule) encapsulated in a lipid nanoparticle (LNP). In some embodiments, the compositions or formulations are capable of increasing the stability of the nucleic acid (e.g. a RNA molecule), encapsulated in the LNP. In some embodiments, the compositions or formulations described herein have an increased stability after a process of drying, storage, and reconstitution (DS&R), compared to a control composition or formulation without the DS&R process. For example, such stability increasing may result in retaining structure and/or activity of the nucleic acid after storing the compositions or formulations at a certain temperature for a time period. In some embodiments, at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% activity of the nucleic acid is retained in the compositions or formulations described herein after storage at room temperature (e.g., about +20° C. to about +30° C., such as 25° C.) for a certain time period, such as at least one day, two days, three day, one week, ten days, two weeks, three weeks, one month, two months, three months, four months, five months, six months, one year, or longer. In some embodiments, the compositions or formulations retain at least 80% to 100% activity of the nucleic acid, encapsulated in the LNP, after storage at room temperature for at least one week or one month.

In one aspect is provided a composition comprising a RNA molecule and a lipid nanoparticle (LNP). In some embodiments, the composition comprises a RNA molecule and a lipid nanoparticle (LNP), wherein the RNA molecule is encapsulated in the LNP, wherein the composition is in the form of sugar glass. In some embodiments, the RNA molecule is a self-amplifying RNA (samRNA). In some embodiments, the RNA molecule is a messenger RNA (mRNA), a small interfering RNA (siRNA), a short hairpin RNAs or small hairpin RNA (shRNA), a microRNA (miRNA), a miRNA inhibitor (antagomirs/antimirs), or a messenger-RNA-interfering complementary RNA (micRNA).

In some embodiments, the RNA molecule described herein encodes a polypeptide or a fragment thereof. In some embodiments, the RNA molecule described herein encodes a viral polypeptide or a fragment thereof. In some embodiments, the viral polypeptide is or is derived from a spike protein of a virus. In some embodiments, the virus is a coronavirus (e.g., SARS-COV-2) or an influenza virus.

In some embodiments, the RNA molecule described herein is a vaccine against a virus comprising the viral polypeptide or fragment thereof to be administered to a subject in need of.

In some embodiments, the composition described herein further comprises a second RNA molecule encapsulated in the LNP. In some embodiments, the composition described herein further comprises more than one additional RNA molecule encapsulated in the LNP.

In some embodiments, the composition described herein is in the form of sugar glass. In some embodiments, the sugar glass comprises one or more of sucrose, glucose, galactose, fructose, trehalose, maltose, and other suitable sugar types. In some embodiments, the sugar glass comprises trehalose.

In some embodiments, the composition described herein further comprises a PEG molecule, an ionizable lipid, and/or a structural lipid.

In some embodiments, the LNP comprises an ionizable lipid. For example, the ionizable lipid may comprise one or more of DLin-MC3-DMA, C12-200, ALC-0315, A9, and SM-102.

In some embodiments, the PEG molecule comprises one or more of polyethylene glycol 2000 (PEG 2000), DMG-PEG2000 (polyethylene glycol 2000 dimyristoyl glycerol), ALC-0159 (2 [(polyethylene glycol)-2000]-N,N-ditetradecylacetamide) and DSPE-PEG2000. In some embodiments, the PEG molecule comprises DSPE-PEG2000.

In some embodiments, the structural lipid described herein comprises one or more of cholesterol, 1,2-distearoyl-sn-glycero-3-phosphocholine (DSPC), 1,2-distearoyl-sn-glycero-3-phosphorylethanolamine (DSPE), and DSPE derivatives [including 1, 2-distearoyl-sn-glycero-3-phosphoethanolamine-Poly(ethylene glycol) (DSPE-PEG), such as DSPE-PEG2000 or DSPE-PEG2K].

In some embodiments, the composition described herein comprises a structural lipid, an ionizable lipid and a PEG molecule in a molar ratio of about 27%-about 40% ionizable lipid, about 45%-about 70% structural lipid and about 0.1%-about 3% PEG molecule. In some embodiments, the composition described herein comprises a molar ratio of about 35%-about 37% ionizable lipid, about 62%-about 64% structural lipid and about 0.3%-about 1% PEG molecule. In some embodiments, the composition described herein comprises a molar ratio of about 36.5% ionizable lipid, about 62% structural lipid and about 0.5% PEG molecule.

In some embodiments, the composition described herein comprises at least one type of sugar. In some embodiments, at least one type of sugar is present in the composition in amount of about 1%-about 60% (w/v), about 1%-about 50% (w/v), about 5%-about 50% (w/v), about 5%-about 20% (w/v), about 5%-about 10% (w/v), about 10%-about 60% (w/v), about 10%-about 50% (w/v), about 10%-about 20% (w/v), or any sub-value and sub-range there between, including endpoints. In some embodiments, at least one type of sugar is present in the composition in amount of about 1%, 5%, 10%, 12%, 15%, 17%, 20%, 25%, 30%, 35%, 40%, 45%, 50%, 55%, 60% (w/v), or more, including all sub-values and sub-ranges there between, including endpoints. In some embodiments, at least one type of sugar is present in the composition in amount of about 5%-about 20% (w/v). In some embodiments, at least one type of sugar is present in the composition in amount of about 12%-about 17% (w/v). In some embodiments, at least one type of sugar is present in the composition in amount of about 15% (w/v).

In some embodiments, the composition described herein comprises any suitable percentage of the water content in the sugar glass. In some embodiments, the water content in the sugar glass is about 0.01 to 100 g·water/g·solid, including all sub-values and sub-ranges there between, including endpoints. In some embodiments, the water content in the sugar glass is about 0.1 to 10, 0.1 to 5, 0.1 to 3, 0.5 to 10, 0.5 to 5, 0.5 to 3, 1 to 10, 1 to 5, or 1 to 3 g·water/g·solid, including all sub-values and sub-ranges there between, including endpoints.

In some embodiments, the composition described herein has an increased stability compared to a similar composition not in the form of sugar glass. The increased stability may be, e.g., at least about 10%, 20%, 30%, 40%, 50%, 60%, 70%, 80%, 90%, 100%, 150%, 200%, 250%, 300%, 350%, 400%, 450%, 500%, 600%, 700%, 800%, 900%, 1000%, 2000%, 5000%, 10000%, or more, including all sub-values and sub-ranges there between, including endpoints.

In some embodiments, the composition described herein has an increased stability at a non-freezing temperature, or the room temperature.

In some embodiments, the composition described herein is capable of being reconstituted after a time period of storage at the non-freezing temperature or room temperature for administration to a subject in need of. The time period of storage may be, e.g., about 1 day, 3 days, one week, 10 days, two weeks, three weeks, one month, or longer.

In some embodiments, the compositions or formulations, prior to administering to the subject, are stored at a certain temperature for a time period, while retaining at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% activity of the nucleic acid, encapsulated in the LNP in the compositions or formulations. In some embodiments, the storage temperature is room temperature (e.g., about +20° C. to about +30° C., such as 25° C.). In some embodiments, the time period of storage is at least one day, two days, three day, one week, ten days, two weeks, three weeks, one month, two months, three months, four months, five months, six months, one year, or longer. In some embodiments, the compositions or formulations retain at least 5%, 10%, 20%, 30%, 40%, 50%, 60%, 70%, 80%, 90%, or 100% activity of the nucleic acid, encapsulated in the LNP, after storage at room temperature for, e.g., at least one week or one month, prior to administering to the subject.

In some embodiments, the composition described herein further comprises an agent capable of facilitating the encapsulation. The agent may, e.g., comprise P188 or HES. In some embodiments, the concentration of the agent in the composition is about 1%, 3%, 5%, 10%, 15%, 20%, 25%, 30%, 35%, 40%, or more, including all sub-values and sub-ranges there between, including endpoints.

In some embodiments, the composition described herein has undergone vitrification. For example, the composition may be vitrified into a sugar glass status.

In another aspect is provided a formulation of the composition described herein. In some embodiments, the formulation further comprises a pharmaceutically acceptable excipient.

In another aspect is provided a vial, ampoule, or pre-filled syringe containing a sugar glass composition or formulation described herein.

In another aspect is provided a microneedle device, a microarray, or similar devices for administering the compositions or formulations described herein. In some embodiments, the microneedle device comprises (a) the composition or the formulation described herein; and (b) a substrate comprising a sheet and a plurality of microneedles extending therefrom, each of said microneedles comprising a tip, a base, a hinge at the base connecting the microneedle to the sheet, and a well comprising the dehydrated composition.

In another aspect is provided a reconstituted composition for administration to a subject in need of, comprising the composition or the formulation described herein. In some embodiments, the reconstituted composition is in the vial, ampoule, or pre-filled syringe described herein.

In another aspect is provided a method of increasing stability of the compositions or formulations described herein, e.g., by vitrification into sugar glass. In some embodiments, the method is a method of increasing stability of an RNA molecule in a composition, comprising formulating the RNA molecule with LNPs and forming the formulation into a sugar glass composition. In some embodiments, the sugar glass composition comprising a composition or formulation described herein. In some embodiments, the sugar glass is formed by vitrification. In some embodiments, the increased stability is a stability at a non-freezing temperature or the room temperature.

In another aspect is provided a method of producing the compositions or formulations described herein by vitrification into sugar glass. In some embodiments, the method is a method of producing a compositions or formulations described herein for administering to a subject in need of. In some embodiments, the method comprises i) vitrifying the composition or formulation into sugar glass; and ii) after a time period of storage at a non-freezing temperature or the room temperature, reconstituting the composition for administering.

In another aspect is provided a method of generating an immune response in a subject, or vaccinating a subject with the compositions or formulations described herein. In some embodiments, the method is a method of generating an immune response a subject in need thereof, comprising administering a therapeutically effective amount of the composition or the formulation described herein.

In another aspect is provided a method of treating a virus-related disease or disorder in a subject by administering the compositions or formulations described herein. In some embodiments, the compositions or the formulations described herein are reconstituted, after a time period of storage at a non-freezing temperature or the room temperature, prior to the administering. In some embodiments, the compositions or formulations, prior to administering to the subject, are stored at a certain temperature for a time period, while retaining at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% activity of the nucleic acid, encapsulated in the LNP in the compositions or formulations. In some embodiments, the storage temperature is room temperature (e.g., about +20° C. to about +30° C., such as 25° C.). In some embodiments, the time period of storage is at least one day, two days, three day, one week, ten days, two weeks, three weeks, one month, two months, three months, four months, five months, six months, one year, or longer. In some embodiments, the compositions or formulations retain at least 80% to 100% activity of the nucleic acid, encapsulated in the LNP, after storage at room temperature for at least one week or one month, prior to administering to the subject.

In another aspect is provided a method for vitrification into a sugar glass in an ampoule, vial or prefilled syringe. In another aspect is provided ampoules, vials or prefilled syringes comprising the compositions and/or formulations described herein. In another aspect is provided a method of using the ampoules, vials or prefilled syringes comprising the compositions and/or formulations, for example, to treat an illness.

### BRIEF DESCRIPTION OF THE DRAWINGS

FIG. 1 is a graph schematically illustrating a DNA construct of template DNA (plasmid pORF57-kan-T7-VEEV-HA) for IVT mRNA.

FIG. 2 is a set of graphs illustrating mRNA qualitative analysis results by agarose gel and Agilent BioAnalyzer of the produced VEEV-HA CA/09 construct.

FIG. 3 is a set of graphs illustrating a DLS (Dynamic Light Scattering) analysis result of examples of samRNA lipid nanoparticles (LNPs).

FIG. 4 is a set of graphs illustrating a CryoElectron microscopy analysis result of examples of samRNA LNPs. The top panel shows a representative image of LNPs and the bottom panel shows a zoom-in view of a single LNP.

FIG. 5 is a set of graphs illustrating the result of fracture analysis of LNPs dried into a sugar glass state.

FIG. 6 is a graph illustrating the result of PAGE analysis of HA produced by samRNA LNP.

FIG. 7 is a set of graphs illustrating immunogenicity of samRNA-containing LNPs in Sprague Dawley rats, delivered by IM Injections. The top panel shows the ELISA titers under each condition. The bottom panel shows the HAI titers under each condition.

FIG. 8 is a set of graphs illustrating a DLS analysis result (the top panel) of samRNA-containing LNPs, after being concentrated, printed on VaxiPatch, and eluted from VaxiPatch and a measurement (the bottom panel) of the activity of the LNPs on 293T cells.

FIG. 9 is a set of graphs illustrating a measurement of the activity of the samRNA-containing LNPs after elution from dried sugar glasses on VaxiPatch.

FIGS. 10A-10E are a set of graphs illustrating DLS and zeta potential of different LNPs, with or without reconstitution, using different drying buffers (DBs).

FIG. 11 is a graph illustrating the plate layout of 293T cells for transfection.

FIG. 12 is a set of graphs illustrating fluorescence of VEE-EGFP replicon mRNA in dried and reconstituted C12-200 LNPs under conditions of using DB1, DB2, DB3 or DB4.

FIG. 13 is a graph comparing fluorescence measured from the data in FIG. 12.

FIG. 14 is a set of graphs illustrating an example of CryoEM image (the left panel) and an example of light microscopy (the right panel) of dried and reconstituted samRNA LNPs, formulated into sugar glass (the right panel).

FIG. 15 is a set of graphs illustrating the loss of water content, measured by the weight of water content in the sugar glass solid (the top panel A) or the ratio of the weight of water content to the weight of the sugar glass solid (the bottom panel B) through time in the sugar glass structure.

FIG. 16 is a set of graphs illustrating EGFP fluorescence intensity of samRNA-containing LNPs, with or without reconstitution, in a similar experimental setting as in FIG. 13. The left panels shows the microscopic images for fluorescence for positive control (SF; the top image), LNPs (the middle image), and reconstituted LNPs (the bottom image). The bars in the right panel, from left to right, represent positive control (SF), LNP, and reconstituted LNPs under conditions of using DB1, DB2, DB3 or DB4.

FIG. 17 is a set of graphs illustrating the stability at room temperature of examples of samRNA-containing LNPs. Freshly made LNPs (the top panels) or LNPs stored at 25° C. for one week (the bottom panels) were compared for their activity by DLS.

FIG. 18 is a set of graphs illustrating the stability at room temperature of examples of samRNA-containing LNPs. Microscopic images (the top panels) were used to compare GFP fluorescence by LNPs and reconstituted LNPs after drying and storage for one week at room temperature. The fluorescence intensities were read and compared (the bottom panel). The columns from left to right represent StemFect (SF), as an external positive control with the same amount of mRNA, and DSPE-PEG2k LNPs, either without reconstitution or being reconstituted under different conditions.

FIG. 19 is a bar chart comparing the HA expression levels by positive control or LNPs reconstituted in the condition of using DB1.

FIGS. 20A and 20B are a set of graphs showing ImageXpress™ Pico fluorescence measurements for total 293T cell numbers (FIG. 20A) and EGFP positive 293T cell numbers (FIG. 20B) after transfection with different LNP formulations. The LNP formulation includes the ionizable lipid C12-200, DOPC, cholesterol (31:11:44), 0.1% DSPE-PEG2K and EGFP samRNA. Each LNP batch included an ethanol solution of lipid mixture at 10 mg/mL and an EGFP-samRNA solution at 116 μg RNA/mL in 25 mM sodium acetate buffer (pH 4). LNPs were assembled using NanoAssemblr at 0.25 mL of lipid solution and 0.75 mL of the RNA solution using the flow rate of 2 mL/min. Ethanol was removed, sodium acetate buffer was exchanged with 15% trehalose in PBS using Amicon centrifugal filter units (100 kDa), and LNPs were concentrated to 125 μL. RNA was quantified by Qubit 3.0 Fluorometer to match RNA concentration prior to addition to the cells. FIG. 20A represents the LNP formulation formulated in 15% trehalose followed by drying, storage at 25° C. and reconstitution in PBS (“A”), whereas “B-7.5” represents the same LNP formulation with the addition of 7.5 μL of DOPC/cholesterol liposomes as excipient after drying, storage and reconstitution (DS&R). For controls shown on the left, the “NTC” is the non-treatment control, the “positive control” is the same amount of EGFP samRNA transfected using Stemfect®, and “LNPs” refers to the LNPs prepared as described above but without DS&R. FIG. 20A shows these treatments are not toxic to cells. FIG. 20B shows the same controls but shows this formulation of LNPs with 20× reduction of PEG from the standard 2% to 0.1% poorly transfect 293T cells unless they are subjected to DS&R.

FIG. 21 is a graph showing the activity of DS&R samRNA Influenza Hemagglutinin (HA) with the same LNPs with different excipient conditions as shown in FIGS. 20A and 20B. “A” represents the LNP formulation without liposomes as excipient after DS&R. “B-1” represents the LNP formulation with 15% trehalose and 1 μL of DOPC/cholesterol liposomes after drying, storage and reconstitution (DS&R). “B-2.5” represents the LNPs formulated with 15% trehalose and 2.5 μL of DOPC/cholesterol liposomes after DS&R. Again, the “positive control” refers to the same amount of EGFP samRNA transfected with Stemfect®.

FIG. 22 is a graph showing gel electrophoresis images to show that HA samRNA, encapsulated in LNPs, is intact after one week storage at 25° C. LNPs were prepared as in FIGS. 20A, 20B, and 21. “0.1 pre” and “0.1 post” refer to 0.1% DSPE-PEG2K before and after DS&R. “0.5 pre” and “0.5 post” refers to 0.5% DSPE-PEG2K before and after DS&R. HA samRNA was extracted from LNPs using 6 volumes of ethanol and vortexing for 30 seconds, followed by an additional 6 volumes of ethanol and an additional 30-second vortexing. The mixture was then centrifuged at 14,000×g for 10 minutes to pellet the RNA. The separated RNA was then suspended in PBS and run on RNA Flash Gels in a Lonza electrophoresis/imaging system.

FIG. 23 is a set of drawings depicting an example for construct design for mRNA that can be used in LNP formulations or vaccines such as described herein. The top panel shows an example of designing mRNA structure for the formulations or vaccines. The bottom panel shows an example of an LNP structure.

FIG. 24 is a diagram showing an example process for making SM-102 self-amplifying mRNA LNPs and the final formulation that can be dried.

FIG. 25 is a set of pictures showing an example of LNP formulations in the form of sugar glass in a vial. The top panel shows a picture of a glass vial containing an example of LNP formulation, with blue dye added to show the dried sugar glass material in the bottom of the vial. The bottom panel shows a picture of an example of the reconstituted formulation in the vial with 2.5 mL PBS, resulting in, for example, five 0.5 mL doses of the LNP formulation, prior to administration.

FIG. 26 is a set of cryo-electron micrograph images of example LNP formulations that include different ionizable lipids (e.g., ALC-0315 or SM-102 lipids). The right panel shows the SM-102 LNP formulations after DS&R.

FIG. 27 is a graph showing that an example of SM-102-containing LNP formulations is stable as sugar glass in glass vials after drying, storage at room temperature and reconstitution (DS&R), maintaining up to 100% activity of the samRNA in the LNP formulation after DS&R, under different conditions 1-4. Control conditions are shown in the leftmost two lanes. As a representation of the samRNAs activity, the expression levels of the HA proteins encoded by the samRNAs in transfected 293T cells were measured and compared in vitro.

FIG. 28 is a depiction showing the analytical composition of LNP formulations that include either mRNA (pink trace, with a higher peak for each of SM-102, cholesterol, and DSPC) or an example of a self-amplifying mRNA (samRNA; black trace, with a lower peak for each of SM-102, cholesterol, and DSPC) encoding HA described herein.

FIG. 29 is a set of bar charts comparing the activity before and after the DS&R process described herein for the exemplary samRNA replicon and LNP formulations with lipids used in the COVID-19 vaccine produced by either Pfizer (the top panel) or Moderna (the bottom panel). EGFP samRNA was used in each formulation and the florescence intensity of EGFP was measured and compared, as an index for RNA stability, for controls (“NTC” and “StemFect-samRNA”) or LNP formulation before (“LNP1” and “LNP2”) and after (“DS&R LNP1” or “DS&R LNP2”) the DS&R process.

FIG. 30 is a set of chats comparing the particle size (the top panel) and the zeta potential (the bottom panel) of the exemplary Pfizer lipid-LNP formulation before and after DS&R, by DLS analysis. In the top panel, the left-side trace refers to LNP before DS&R, while the right-side trace refers to LNP after DS&R. In the bottom panel, the inner trace refers to LNP before DS&R, while the outer trace refers to LNP after DS&R.

FIG. 31 is a chart showing the HPLC analysis of the Pfizer lipids in an exemplary samRNA-LNP formulation.

FIG. 32 is a graph showing the gel electrophoresis of the mRNA expressing HA in the LNPs before and after DS&R. The mRNA of exemplary LNP formulations with Pfizer (“I”) or Moderna (“II”) lipids were extracted before (“Day 0”) or after DS&R (which includes 7 days of storage at room temperature) and compared.

FIG. 33 is a graph showing the gel electrophoresis of the RNA components in an exemplary Pfizer lipid-LNP formulation before and after DS&R. RNAase protection assay was used to test the stability of RNAs in the formulation under each condition.

FIGS. 34A-34C is a set of chats showing the HPLC analyses of the Pfizer lipids in exemplary samRNA-LNP formulations. Extracted LNPs (FIG. 34A), 1×RNA LNPs after DS&R (FIG. 34B), and 1.5×RNA LNPs after DS&R (FIG. 34C) are shown. In FIG. 34B, the outer trace with higher peaks for P-188, ALC-0315 and cholesterol refers to the formulation in vial, compared to the Cap 3 condition. In FIG. 34C, the outer trace with higher peaks for P-188, ALC-0315 and cholesterol refers to the formulation under the Cap 3 condition, compared to the formulation in vial.

FIG. 35 is a chat showing the HPLC analysis of the extracted Moderna lipids in exemplary samRNA-LNP formulations.

### DETAILED DESCRIPTION

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The present disclosure generally relates to compositions and formulations of nucleic acid molecules, for example, DNA and RNA, with improved stability, and methods of making and using the same. The compositions and formulations can comprise lipid nanoparticles (LNPs). For example, some embodiments relate to compositions and formulations of RNA molecules that can be used to generate an immune response or for any other therapeutic purpose. Such compositions and formulations can be stable for periods of time at room temperature. The compositions and formulations can be vitrified or in the form of a sugar glass. The vitrified or sugar glass compositions and formulations can be packaged and/or utilized with any suitable apparatus. For example, the vitrified or sugar glass material can be packaged in a vial, ampoule, syringe or other suitable container, and can be reconstituted prior to use. Upon reconstitution, the material can be administered in any suitable manner, for example, via intramuscular (IM) administration. In another example, the vitrified or sugar glass material can be administered topically. In some instances the material can be packaged in “needle” arrays, such as microneedle arrays, which can be used for example for topical administration of the material.

In some aspects, the present disclosure describes, e.g., methods to stabilize any nucleic acids (e.g., LNP mRNA vaccines) at room temperature. The methods involve vitrification to form a sugar glass using any suitable sugar, including e.g., trehalose, others described herein or any other suitable sugar. These methods can be used to develop mRNA vaccines directed towards a long list of infectious agents whether in traditional vials delivered by needle and syringe, delivered orally, intranasally, or in microneedle arrays such as VaxiPatch. The mRNA vaccines can encode single proteins or multiple proteins and can be self-amplifying mRNAs. mRNA vaccines delivered on VaxiPatch may enable rapid immune response and function as room temperature stable Shelter-In-Place vaccines during a pandemic. Room temperature stable mRNA vaccines will enable renewed health initiatives within the USA, and also globally.

In some embodiments, the nucleic acid lipid nanoparticle composition can be stored at a temperature above freezing, for example at temperature above 3° C. (e.g., approximately 4° C.), or higher temperatures (e.g., 4° C. to 35° C.) such as at room temperature (at about +20° C. to about +30° C./or any sub-value or sub-range therein, including endpoints). In some examples, the nucleic acid LNP composition is in a vitrified form (e.g., in a sugar glass) and is stored and stored at a temperature above freezing, e.g., at room temperature, such as about +20° C. to about +30° C. In some examples, the nucleic acid composition is in a dehydrated and vitrified form (e.g., in a sugar glass) and is stored and stored at a temperature above freezing, e.g., at room temperature, such as about +20° C. to about +30° C. In some embodiments, drying and vitrification, either alone or in combination, increases stability of the replicon composition at a temperature above freezing, e.g., e.g., room temperature such as about +20° C. to about +30° C. Such stability increase may be, e.g., about an increase of at least 10%, 20%, 30%, 40%, 50%, 60%, 70%, 80%, 90%, 100%, 150%, 200%, 250%, 300%, 350%, 400%, 450%, 500%, 600%, 700%, 800%, 900%, 1000%, 2000%, 5000%, 10000%, or more (and all sub-values and sub-ranges there between, including endpoints), compared to a control nucleic acid composition not dried and/or vitrified. Such increase in stability may be measured by various assays, such as the translation of the mRNA in the replicon composition, the function of the translated protein/polypeptide by the mRNA, etc., after a certain time period of storage. The term “stability” generally refers to the ability of the LNPs, when encapsulated with nucleic acids described herein, to remain active after a certain time period of storage, transportation, or other process prior to administration to a subject. Such stability may refer to stability at each and every process after encapsulating nucleic acids with LNPs, such as stability during the time period from forming formulations of encapsulated LNPs to drying, from drying to vitrification, from vitrification to storage, from storage to reconstitution, and/or from reconstitution to administration to a subject. Such stability may refer to maintaining the structure of LNPs and/or sugar glass from degradation, maintaining the structure of nucleic acids from degradation, maintaining the activity of nucleic acids (e.g., self-amplifying RNA) to amplify, to transcribe a RNA molecule, and/or to translate a polypeptide, and/or maintaining the activity of the transcribed RNA molecule or the translated polypeptide (e.g., being able to bind to and infect a cell). Such stability may be measured by various methods to test the structure and/or the function/activity of nucleic acids and/or LNPs.

Prior to December 2019, adjuvanted protein subunit vaccines typified by the 2017 FDA approval of Shingrix® from GSK with the AS01b adjuvant and a subunit protein produced in CHO cells held sway as the apex of approvable protein based vaccine technology. Sanofi had recently purchased Protein Sciences to obtain FluBlok®, a recombinant influenza hemagglutinin (rHA) subunit vaccine produced in insect cells. Novavax was developing a rHA insect cell produced protein based vaccine with their Matrix-MTM adjuvant.

SARS-COV-2 causing COVID-19 changed that paradigm. Not a single mRNA vaccines or Adenovirus vectored vaccine had won FDA approval. But in a pandemic time was of the essence and, based on previous coronavirus work with SARS and MERS, the spike protein of SARS-COV-2 offered an accessible vaccine target.

mRNA vaccine work at Moderna, BioNTech and CureVac aimed at individualized cancer vaccines had not produced a marketed product. Work on an mRNA vaccine for influenza with BioNTech partnered with Pfizer offered promise, but the FDA approval process was uncertain. The FDA approves the release of each lot of vaccine produced commercially. To accomplish this lot release the sponsor and the FDA have to agree on a potency test for the vaccine. No gene based vaccine (DNA, RNA or viral vector) had been FDA approved. Another complication was the delivery of DNA, mRNA and viral vectors.

After twenty years of frustration with gene delivery of virus vectored vaccines and DNA vaccines with lipids including DOTMA, a non-vaccine gene delivery of a small inhibitory (siRNA) product was approved by the FDA in 2018. This double stranded siRNA was formulated into lipid nanoparticles (LNPs) with two structural lipids, cholesterol and 1,2-distearoyl-sn-glycero-3-phosphocholine (DSPC), a PEGylated lipid (α-(3′-{[1,2-di (myristyloxy)propanoxy] carbonylamino} propyl)-ω-methoxy, polyoxyethylene (PEG2000 C-DMG)), and the ionizable lipid (6Z,9Z,28Z,31Z)-heptatriaconta-6,9,28,31 tetraen-19-yl-4-(dimethylamino) butanoate (DLin-MC3-DMA). Chemically, these products are surprisingly well defined. The first product was patrisiran (ONPATTROR) and is a treatment for hereditary transerythyretin amyloidosis. ONPATTRO uses the DLin-MC3-DMA as the ionizable lipid. Pioneering lipid delivery work was performed by Pieter Cullis and colleagues at the University of British Columbia, resulting in several spin out companies such as Precision NanoSystems and Acuitas. Moderna developed its own ionizable lipids.

But delivery of larger single-stranded mRNA molecules remained inefficient. Intense competitive work continued in the development of ionizable lipids to improve upon DLin-MC3-DMA. The issues were to learn how to formulate the mRNA molecule so it was: (1) not immediately destroyed by ubiquitous RNAse enzymes, (2) taken up by cells, (3) transported within the cell to endosomes, and (4) released from the endosome and into the cytoplasm in a form capable of translation into protein. For the current Emergency Use Authorization mRNA COVID-19 vaccines Pfizer/BioNTech used ionizable lipids from Acuitas Therapeutics of Vancouver, BC.

The LNPs of the Pfizer/BNT162b2 and Moderna mRNA-1273 COVID mRNA vaccines granted FDA Emergency Use Authorization (EUA) in December 2020 contain four components: the structural lipids (1) cholesterol and (2) DSPC, (3) a PEGylated lipid such as PEG2000-DMG and (4) an ionizable lipid. Improvements and variations are often seen in the fourth component, the ionizable lipid. The structures of the ionizable lipids DLin-MC3-DMA (Biofine International, Blaine, WA), and C12-200 (CordenPharma, Plankstadt, Germany), ALC-0315 (Acuitas, Vancouver, BC) found in the Prizer/BNT162b2 vaccine, and SM-102 found in mRNA-1273 vaccine are shown below. Other LNP functions such as targeting, stability, reduced toxicity and may involve additional components.

The ionizable lipids (4-hydroxybutyl) azanediyl)bis(hexane-6,1-diyl)bis(2-hexyldecanoate), ALC-0315, and A9 were described by Acuitas. SM-102 was described by Moderna.

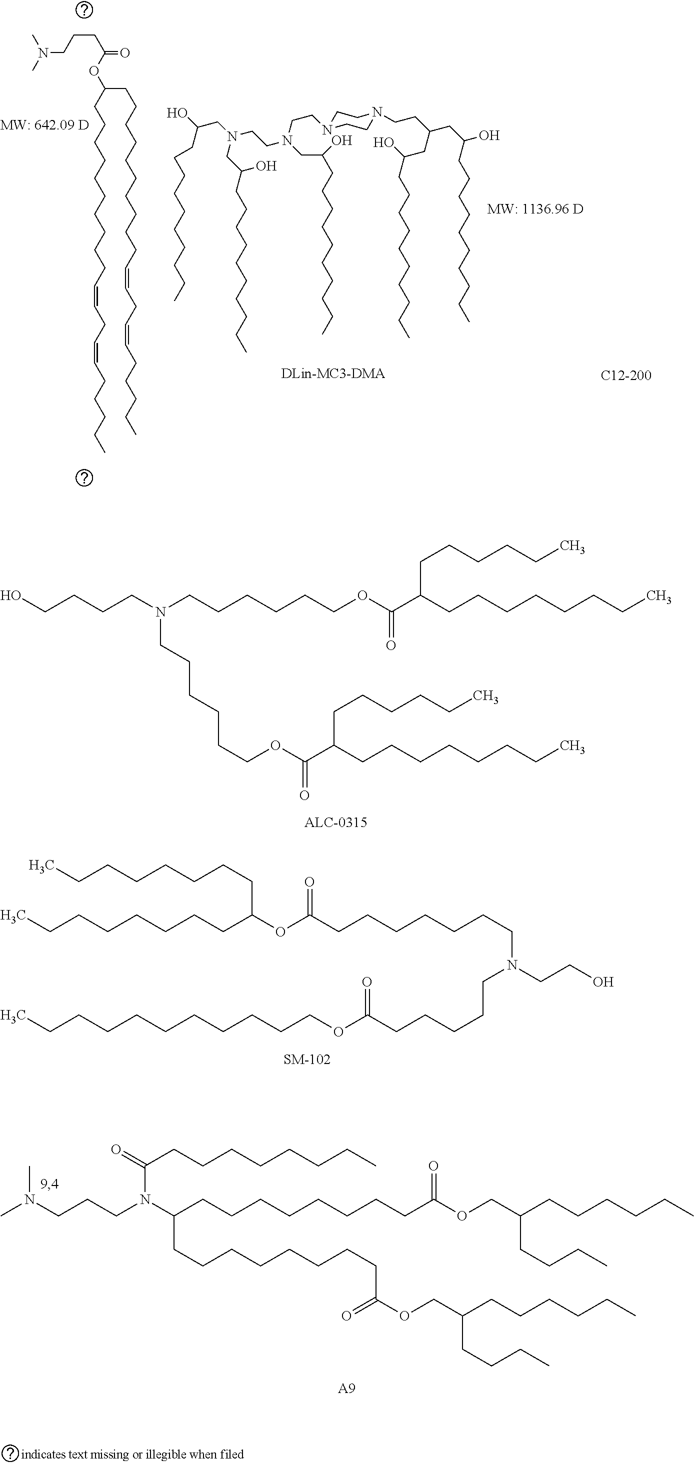

Other lipids useable in the LNPs described herein may include lipid compounds known to a skilled artisan, including stereoisomers, pharmaceutically acceptable salts or tautomers thereof, which can be used alone or in combination with other lipid components such as neutral lipids, charged lipids, steroids (including for example, all sterols) and/or their analogs, and/or polymer conjugated lipids to form lipid nanoparticles for the delivery of therapeutic agents.

As noted above, the lipid nanoparticles can be used to deliver any desired nucleic acid molecule, including various types of RNA. It should be understood that in the various embodiments and aspects described herein, any type of RNA is contemplated and can be utilized. In some instances, the lipid nanoparticles are used to deliver nucleic acids such as antisense oligonucleotides, small interfering RNAs (siRNAs), self-amplifying RNAs (samRNAs), short hairpin RNAs or small hairpin RNAs (shRNAs), messenger RNAs, plasmid DNAs, microRNAs (miRNAs), miRNA inhibitors (antagomirs/antimirs), messenger-RNA-interfering complementary RNA (micRNA), DNAs, multivalent RNAs, dicer substrate RNAs, complementary DNAs (cDNAs), etc.

Examples of lipids useable in the LNP described herein may include lipid compounds disclosed in U.S. Pat. Nos. 11,040,112, 10,723,692, 10,221,127, 10,166,298, 10,106,490, 9,738,593, 9,737,619 (e.g., in Table 1 of these patents), U.S. Pat. Nos. 9,675,668, 9,447,164, 9,301,993, 9,295,689, and 8,754,062, each of which is incorporated by reference herein to its entirety.

Examples of lipids useable in the LNP described herein may include lipid compounds disclosed in U.S. Pat. Nos. 10,022,436 and 10,363,303 and PCT Application Nos. PCT/US2017/045161 and PCT/US2020/044196, each of which is incorporated by reference herein to its entirety.

In January 2020, the sequence of SARS-COV-2 was published. Organizations globally started work on new vaccines for COVID-19. On Mar. 11, 2020 the World Health Organization (WHO) declared COVID-19 a pandemic. In March 2020, the Pfizer/BioNTech/Acuitas influenza vaccine partnership officially shifted to deliver the mRNA of the spike protein of SARS-COV-2.

The US Government Operation WARP Speed was officially announced on May 15, 2020. The Pfizer/BioNTech program escued R&D WARP support but joined WARP in a supply agreement. WARP supported R&D at Moderna (mRNA vaccine), Johnson and Johnson and AstraZeneca/Oxford (both Adenovirus vaccines), and Novavax and Sanofi/GlaxoSmith Kline (both adjuvented recombinant spike protein vaccines produced in insect cells). In December 2020 Emergency Use Authorization was granted by the FDA to the Pfizer/BioNtech vaccine. In a 0.3 mL dose, the vaccine contained 30 mcg of pseudouridine (Kariko) modified mRNA encoding the viral spike(S) glycoprotein of SARS-COV-2. Each dose also contains the lipids: (1) an ionizable lipid from Acuitas ALC-0315 (4-hydroxybutyl) azanediyl)bis(hexane-6,1-diyl)bis(2-hexyldecanoate), (2) a PEGylated lipid (2 [(polyethylene glycol)-2000]-N,N-ditetradecylacetamide), and the structural lipids 1,2-distearoyl-sn-glycero-3-phosphocholine (DSPC) and cholesterol. Also included is sodium chloride and sodium phosphate buffer.

While in December 2020 the FDA granted Emergency Use Authorization to the Pfizer/BioNTech and Moderna mRNA vaccines, as of Aug. 1, 2021 the FDA has only granted one additional EUA. That EUA is to the Johnson and Johnson human Adenovirus type 26 replication defective vaccine. The adjuvanted subunit protein vaccines, the apex of subunit protein vaccines prior to COVID-19, from Novavax and Sanofi/GSK are not yet approved. Phase 3 clinical trials of the two mRNA vaccines showed safety and 95% protection from COVID-19 in clinical trials. These stunningly positive results overwhelmed doubts and concerns of mRNA vaccines and any criticism of potency testing methodologies. With the Adenovirus vaccines showing more production and clinical concerns than the mRNA vaccines, the clear winner is the new mRNA vaccine technology.

However, the mRNA technology was so new, the initial roll-out of the Pfizer/BioNTech COVID-19 vaccine in the first quarter of 2021 required dry ice shipment and storage at −80° C. Pfizer/BioNTech had not had the time required to perform the necessary stability studies. Shipment and storage today has improved, but still requires freezing at −20° C.

In the second quarter of 2020, supply, delivery and administration of COVID vaccines has taken center stage along with concerns of SARS-COV-2 variants. Vaccination rates beyond the US and Western Europe are lagging. There is a global concern for the unvaccinated population, anywhere globally, for this population represents incubators for the development of ever more infectious SARS-COV-2 variants. Delivery and distribution of vaccines is a global problem long complicated by: (1) dependence on reliable electricity to support the vaccine distribution cold-chain, and (2) the limited supply of skilled medical professionals to administer vaccines.

Verndari has developed VaxiPatch, a skin patch vaccine delivery system capable of room temperature distribution and administration by lower skilled professionals and even capable of self-administration. Verndari has an issued patent that describes the Vaxipatch microarray produced by photochemical etching, methods for loading vaccine on the microarray and includes the use of dendrimers to deliver mRNA in a dried state. However, the clinical success of the LNP ionizable lipid technology identifies the LNP technology as preeminent.

U.S. Patent Application Publication Nos. 20120258046 and 20170182081 by Mutzke assigned to Moderna describes free RNA lyophilized with mannose. U.S. Pub No. 20190133950 by Eber, et al., assigned to CureVac describes RNA containing LNPs preserved as a dry powder prepared by spray-freeze drying. U.S. Pub No. 20200069599 by Smith, et al., assigned to Moderna describe lyophilization LNPs with an amphiphilic polymer such as P188, including an LNP containing mRNA. U.S. Pub No. 2020/02921A1 by Karve, et al., assigned to Translate Bio also describes spray drying of mRNA LNPs.

Proof of Concept (POC) of proteins or peptides using the Verndari VaxiPatch microarray platform was shown with Commercial Fluzone® High Dose (GSK), FluBlok® (Sanofi) and Hepatitis B (GSK). Feasibility work was also shown with a live attenuated measles vaccine (the Schwartz/Moraten strain of measles isolated from MMRII and grown on VERO cells, Merck). Verndari published its VaxiPatch microarray system using recombinant HA of influenza produced in CHO cells with and without VAS 1.0, a liposomal excipient designed to mimic AS01b of Shingrix®, see Thomas J. Ellison, George C. Talbott, Daniel R. Henderson, VaxiPatch™, a novel vaccination system comprised of subunit antigens, adjuvants and microneedle skin delivery: An application to influenza B/Colorado/06/2017, Vaccine 38 (2020) 6839-6848, https://doi.org/10.1016/j.vaccine.2020.07.040. Recently, Verndari has shown POC with an FDA Approved Quadivalent vaccine in partnership with a large pharmaceutical company.

VaxiPatch has been designed to deliver a sterile vaccine. Phase 1 VaxiPatch vaccines are made by hand. But VaxiPatch is unique. It has been designed for automated cGMP production. Both prototype and production systems have been designed to make 20 units/minute and 200 units/minute respectively. Both systems are designed to function in an aseptic ISO5 space. The first system requires a large ISO5 space whereas the second system is designed to fit in a generic SKAN Isolator within an ISO7 space. The 10× based on the second system would require a custom isolator also housed in an ISO7 space. Components of VaxiPatch would be brought to automated system as pre-sterilized components. The prototype system operating at 20 units/minute would generate 10,000 units/8 hour shift and would support FDA product approval. Construction of the 10× increased volume line working 2 shifts/day 5 days/week would produce 1 M units/week. Four parallel lines at the same site would produce 4 M units/week or 16 M units/month. Even larger systems can be designed and multiple locations can be considered. The envision is that these larger manufacturing systems would be constructed after FDA Approval using the 20 unit/minute prototype production line described above. These systems could be used for as a platform for production of both protein and mRNA vaccines.

Both the automated systems may include multiple camera and computer aided process controls with reject capabilities. The vaccine print mix can have its own set of in process QA and QC parameters. Both protein and nucleic acid (e.g., mRNA) vaccines can be eluted from printed microarrays for final lot release QA/QC testing.

However, the above-described approaches at formulating nucleic acids in a stable manner at temperatures above freezing has proven to be a formidable challenge. As described more fully below, embodiments herein relate generally surprising and unexpected discoveries of vitrified or sugar glass compositions and formulations of lipid nanoparticles and nucleic acid molecules with both surprising room temperature stability for extended periods of time and with activity after such storage. These embodiments were not predictable in view of the prior approaches. The innovative compositions and formulations can used with the already-mentioned microarray devices (e.g., Vaxipatch™) or with any other storage or delivery device (e.g., ampoules, vials, pre-filled syringes, and the like). The following sections provide details of the various components and methods of the surprising and unexpected compositions and methods.

In short, generally the embodiments herein relate to compositions, components and materials, and methods of making and using the vitrified sugar glass compositions. In some embodiments, the sugar glass compositions and formulations described herein comprise a lipid nanoparticle (LNP) and a nucleic acid (e.g., DNA or RNA) described herein. In some embodiments, the LNPs comprise at least one structural lipid and at least one functional lipid. In some embodiments, the functional lipid includes an ionizable lipid. In some embodiments, the compositions and formulations described herein comprises a first structural lipid, a second structural lipid, an ionizable lipid. In some embodiments, the compositions and formulations can include a PEG molecule. Any suitable PEG molecule can be utilized including for example a PEGylated lipid molecule. Examples of such molecules are described herein. The vitrified sugar compositions can comprise any suitable sugar. The sugar can include those known to the skilled artisan, including for example, trehalose, or any other described herein. Other excipients optionally can be included.

In some embodiments, the first structural lipid comprises any lipid suitable for use herein. In some embodiments, the first structural lipid is 1,2-distearoyl-sn-glycero-3-phosphocholine (DSPC). In some embodiments, the second structural lipid comprises any lipid suitable for use herein. In some embodiments, the second structural lipid is cholesterol. In some embodiments, the first and the second structural lipid are selected from cholesterol, 1,2-distearoyl-sn-glycero-3-phosphocholine (DSPC), 1,2-distearoyl-sn-glycero-3-phosphorylethanolamine (DSPE), and DSPE derivatives [including 1, 2-distearoyl-sn-glycero-3-phosphoethanolamine-Poly (ethylene glycol) (DSPE-PEG), such as DSPE-PEG2000 or DSPE-PEG2K].

In some embodiments, the ionizable lipid comprises any lipid suitable for use herein. In some embodiments, the ionizable lipid is DLin-MC3-DMA, C12-200, ALC-0315, A9, or SM-102. In some embodiments, the ionizable lipid is C12-200.

In some embodiments, the PEG molecule comprises any polyethylene glycol molecule or derivative thereof suitable for use herein. In some embodiments, the PEG comprises polyethylene glycol 2000 (PEG 2000), DMG-PEG2000 (polyethylene glycol 2000 dimyristoyl glycerol), ALC-0159 (2 [(polyethylene glycol)-2000]-N,N-ditetradecylacetamide), DSPE-PEG2000, etc. In some embodiments, the PEG comprises DSPE-PEG2000.

### Potential Usage of the Nucleic Acid-LNP Delivery System

As described herein, embodiments of the lipid nanoparticles (LNPs) of the present invention are particularly useful for the delivery of nucleic acids as described herein. Compared to virus-based vector vaccines, a nucleic acid (e.g., an mRNA) vaccine based on the nucleic acid-LNP delivery system described herein can have multiple advantages, such as, for example, there is no vehicle antigenicity, pre-existent neutralizing antibodies, or virus replication, shedding, or integration into chromosomes in the host.

In one example of preparing an mRNA construct for delivery by Synthesized in vitro Transcription (ivT-mRNA), a reaction mix previously stored at −20° C. may be used, including ATP, GTP, UTP, and CTP (100 mM for each), 10×T7 reaction buffer, 0.5 μg/μl DNA template, and T7 RNA polymerase. The synthesized mRNA chain may be further added with a 5′ Cap and a 3′ poly A tail, prior to purification.

An ivT-mRNA vaccine can be delivered with a stable nucleic acid lipid particle (SNALP). An example of SNALP may contain 10% zwitterionic lipid, 40% cationic lipid, 48% cholesterol, 2% PEGlyated lipid, and self-amplifying RNA.

Such mRNA vaccines may be used to target different diseases or disorders, such as, for example, influenza, diphtheria, tetanus, & acellular pertussis (DTaP), (inactivated) poliovirus, measles, mumps, rubella, meningococcus, Haemophilus Influenzae Type b5, hepatitis B (HepB), varicella (VAR), hepatitis A (HepA), pneumococcal conjugate, rotavirus, human papillomavirus, etc.

Suitable viral polypeptides encodable by the mRNA vaccine may include, e.g., Has, NA, M2, M2e, or other proteins for universal flu, HA for seasonal flu, VP1 for polio virus or HPV, GP or hemagglutinin for measles, capsid C and/or GP E1 & E2 for Hepatitis C virus (HCV), GP for rabies, gag, pol, or other viral proteins for HIV, Ag85A for tuberculosis, MSP1, AMA1, CSP, and/or 85A for malaria, fusion protein F for respiratory syncitial virus (RSV), A2 for visceral leishmaniasis (VL), T. cruzi gp83 for Chagas Disease, Schistosoma japonicum inhibitor apoptosis protein (Ad-SjIAP) for certain viruses, and certain viral proteins for Ebola or Chikungunya viruses. Other mRNA vaccines may be used against infectious diseases, such as influenza, zika, HIV, Ebola, rabies, chikungunya, malaria, genital herpes, Toxoplasma gondii, etc., as reported in Gómez-Aguado et al., Nanomaterials 2020, 10:364; Kowalski et al. Mol. Ther. 2019, 27:710-728; Alameh et al. Messenger RNA-Based Vaccines Against Infectious Diseases. In Current Topics in Microbiology and Immunology; Springer: Berlin/Heidelberg, Germany, 2020; pp. 1-35; and Maruggi et al. Mol. Ther. 2019, 27:757-772, the contents of which are incorporated by reference herein to their entirety. For a summary of nanomaterial delivery systems for mRNA vaccines, such as current mRNA LNPs for SARs-COV-2 clinical trials, see Buschmann et al., Vaccines 2021, 9:65, the contents of which is incorporated by reference herein to its entirety.

In some embodiments, the nucleic acid-LNP delivery system described herein may be used to replace current gene therapies (such as virus-based gene therapies). In some embodiments, the system may be used to deliver a nucleic acid comprising of or capable of translating into a functional RNA molecule (e.g., mRNA, siRNA, miRNA, shRNA, gRNA, or other RNAs described herein) or capable of encoding a functional polypeptide (e.g., a therapeutic protein, an antibody, etc.). In some embodiments, the system may be used to deliver a gRNA. In some embodiments, the system may comprise may be used to deliver a nucleic acid encoding at least one polypeptide in a CRISPR system (e.g., a CRISPR/cas9 or other CRISPR systems). By delivering either a gRNA or a CRISPR-related polypeptide, an example of the nucleic acid-LNP delivery system described herein may induce gene editing through CRISPR systems in cells.

In some embodiments, the nucleic acid-LNP delivery system described herein may be used to produce vaccines for protecting or treating a subject in need thereof, such as a subject infected by or exposed to a pathogen. Examples of pathogens may include chemical compounds, nucleic acids (e.g., DNAs, RNAs, etc.), amino acids (e.g., peptides or proteins), pathogenic organisms (e.g., bacteria, viruses, malignant cells, etc.), etc. Examples of vaccines may include cancer/tumor vaccines, anti-viral vaccines, anti-poison (e.g., anti-Ricin) vaccines, etc.

### Nucleic Acids

As described herein, embodiments of the lipid nanoparticles of the present invention are particularly useful for the delivery of nucleic acids, including, e.g., mRNA, antisense oligonucleotide, small interfering RNA (siRNA), self-amplifying RNA (samRNA), short hairpin RNAs or small hairpin RNA (shRNA), plasmid DNA, microRNA (miRNA), miRNA inhibitors (antagomirs/antimirs), messenger-RNA-interfering complementary RNA (micRNA), DNA, multivalent RNA, dicer substrate RNA, complementary DNA (cDNA), etc. In some embodiments, the nucleic acid comprises any nucleic acid suitable for use herein. In some embodiments, the nucleic acid comprises mRNA, antisense oligonucleotide, small interfering RNA (siRNA), self-amplifying RNA (samRNA), short hairpin RNAs or small hairpin RNA (shRNA), plasmid DNA, microRNA (miRNA), miRNA inhibitors (antagomirs/antimirs), messenger-RNA-interfering complementary RNA (micRNA), DNA, multivalent RNA, dicer substrate RNA, complementary DNA (cDNA), etc. In some embodiments, the nucleic acid comprises mRNA, samRNA, siRNA or shRNA.

In some embodiments, the optional sugar in the compositions and formulations described herein comprises any sugar suitable for use herein. In some embodiments, the optional sugar comprises sucrose, glucose, galactose, fructose, trehalose, maltose, or a combination thereof. In some embodiments, the optional sugar comprises a disaccharide. In some embodiments, the optional sugar comprises a mixture or combination of at least two sugars, such as sucrose and trehalose in a suitable ratio.

In some embodiments, the compositions and formulations described herein comprise an optional liposome. Such optional liposome may have a pre-determined size and/or components suitable for uses herein (e.g., pharmaceutical uses). For a review on liposomes, see Akbarzadeh et al., Nanoscale Res Lett. 2013; 8 (1): 102. In some embodiments, the optional liposome comprises DOPC and/or cholesterol.

### MRNA Molecules

Nucleic acids for use, as described in the present disclosures, may be prepared according to any available technique. For mRNA, the primary methodology of preparation is, but not limited to, enzymatic synthesis (also termed in vitro transcription) which currently represents the most efficient method to produce long sequence-specific mRNA. In vitro transcription describes a process of template-directed synthesis of RNA molecules from an engineered DNA template comprised of an upstream bacteriophage promoter sequence (e.g. including but not limited to that from the T7, T3 and SP6 coliphage) linked to a downstream sequence encoding the gene of interest. Template DNA can be prepared for in vitro transcription from a number of sources with appropriate techniques which are well known in the art including, but not limited to, plasmid DNA and polymerase chain reaction amplification (see Linpinsel, J. L and Conn, G. L., General protocols for preparation of plasmid DNA template and Bowman, J. C., Azizi, B., Lenz, T. K., Ray, P., and Williams, L. D. in RNA in vitro transcription and RNA purification by denaturing PAGE in Recombinant and in vitro RNA syntheses Methods v. 941 Conn G. L. (ed), New York, N.Y. Humana Press, 2012). Transcription of the RNA occurs in vitro using the linearized DNA template in the presence of the corresponding RNA polymerase and adenosine, guanosine, uridine and cytidine ribonucleoside triphosphates (rNTPs) under conditions that support polymerase activity while minimizing potential degradation of the resultant mRNA transcripts. In vitro transcription can be performed using a variety of commercially available kits including, but not limited to RiboMax Large Scale RNA Production System (Promega), MegaScript Transcription kits (Life Technologies) as well as with commercially available reagents including RNA polymerases and rNTPs. The methodology for in vitro transcription of mRNA is well known in the art (see, e.g. Losick, R., 1972, In vitro transcription, Ann Rev Biochem v. 41 409-46; Kamakaka, R. T. and Kraus, W. L. 2001. In Vitro Transcription. Current Protocols in Cell Biology. 2:11.6:11.6.1-11.6.17; Beckert, B. And Masquida, B., (2010) Synthesis of RNA by In Vitro Transcription in RNA in Methods in Molecular Biology v. 703 (Neilson, H. Ed), New York, N.Y. Humana Press, 2010; Brunelle, J. L. and Green, R., 2013, Chapter Five—In vitro transcription from plasmid or PCR-amplified DNA, Methods in Enzymology v. 530, 101-114; all of which are incorporated by reference herein to their entireties).

The desired in vitro transcribed mRNA is then purified from the undesired components of the transcription or associated reactions (including unincorporated INTPs, protein enzyme, salts, short RNA oligos, etc.). Techniques for the isolation of the mRNA transcripts are well known in the art. Well known procedures include phenol/chloroform extraction or precipitation with either alcohol (ethanol, isopropanol) in the presence of monovalent cations or lithium chloride. Additional, non-limiting examples of purification procedures which can be used include size exclusion chromatography (Lukavsky, P. J. and Puglisi, J. D., 2004, Large-scale preparation and purification of polyacrylamide-free RNA oligonucleotides, RNA v. 10, 889-893), silica-based affinity chromatography and polyacrylamide gel electrophoresis (Bowman, J. C., Azizi, B., Lenz, T. K., Ray, P., and Williams, L. D. in RNA in vitro transcription and RNA purification by denaturing PAGE in Recombinant and in vitro RNA syntheses Methods v. 941 Conn G. L. (ed), New York, N.Y. Humana Press, 2012). Purification can be performed using a variety of commercially available kits including, but not limited to SV Total Isolation System (Promega) and In Vitro Transcription Cleanup and Concentration Kit (Norgen Biotek).

Furthermore, while reverse transcription can yield large quantities of mRNA, the products can contain a number of aberrant RNA impurities associated with undesired polymerase activity which may need to be removed from the full-length mRNA preparation. These include short RNAs that result from abortive transcription initiation as well as double-stranded RNA (dsRNA) generated by RNA-dependent RNA polymerase activity, RNA-primed transcription from RNA templates and self-complementary 3′ extension. It has been demonstrated that these contaminants with dsRNA structures can lead to undesired immunostimulatory activity through interaction with various innate immune sensors in eukaryotic cells that function to recognize specific nucleic acid structures and induce potent immune responses. This in turn, can dramatically reduce mRNA translation since protein synthesis is reduced during the innate cellular immune response. Therefore, additional techniques to remove these dsRNA contaminants have been developed and are known in the art including but not limited to scalable HPLC purification (see e.g. Kariko, K., Muramatsu, H., Ludwig, J. and Weissman, D., 2011, Generating the optimal mRNA for therapy: HPLC purification eliminates immune activation and improves translation of nucleoside-modified, protein-encoding mRNA, Nucl Acid Res, v. 39 e142; Weissman, D., Pardi, N., Muramatsu, H., and Kariko, K., HPLC Purification of in vitro transcribed long RNA in Synthetic Messenger RNA and Cell Metabolism Modulation in Methods in Molecular Biology v. 969 (Rabinovich, P. H. Ed), 2013). HPLC purified mRNA has been reported to be translated at much greater levels, particularly in primary cells and in vivo.

A significant variety of modifications have been described in the art which are used to alter specific properties of in vitro transcribed mRNA, and improve its utility. These include, but are not limited to modifications to the 5′ and 3′ termini of the mRNA. Endogenous eukaryotic mRNA typically contain a cap structure on the 5′-end of a mature molecule which plays an important role in mediating binding of the mRNA Cap Binding Protein (CBP), which is in turn responsible for enhancing mRNA stability in the cell and efficiency of mRNA translation. Therefore, highest levels of protein expression are achieved with capped mRNA transcripts. The 5′-cap contains a 5′-5′-triphosphate linkage between the 5′-most nucleotide and guanine nucleotide. The conjugated guanine nucleotide is methylated at the N7 position. Additional modifications include methylation of the ultimate and penultimate most 5′-nucleotides on the 2′-hydroxyl group.

Multiple distinct cap structures can be used to generate the 5′-cap of in vitro transcribed synthetic mRNA. 5′-capping of synthetic mRNA can be performed co-transcriptionally with chemical cap analogs (i.e. capping during in vitro transcription). For example, the Anti-Reverse Cap Analog (ARCA) cap contains a 5′-5′-triphosphate guanine-guanine linkage where one guanine contains an N7 methyl group as well as a 3′-O-methyl group. However, up to 20% of transcripts remain uncapped during this co-transcriptional process and the synthetic cap analog is not identical to the 5′-cap structure of an authentic cellular mRNA, potentially reducing translatability and cellular stability. Alternatively, synthetic mRNA molecules may also be enzymatically capped post-transcriptionally. These may generate a more authentic 5′-cap structure that more closely mimics, either structurally or functionally, the endogenous 5′-cap which have enhanced binding of cap binding proteins, increased half-life, reduced susceptibility to 5′ endonucleases and/or reduced 5′ de-capping. Numerous synthetic 5′-cap analogs have been developed and are known in the art to enhance mRNA stability and translatability (see, e.g., Grudzien-Nogalska, E., Kowalska, J., Su, W., Kuhn, A. N., Slepenkov, S. V., Darynkiewicz, E., Sahin, U., Jemielity, J., and Rhoads, R. E., Synthetic mRNAs with superior translation and stability properties in Synthetic Messenger RNA and Cell Metabolism Modulation in Methods in Molecular Biology v. 969 (Rabinovich, P. H. Ed), 2013).

On the 3′-terminus, a long chain of adenine nucleotides (poly-A tail) is normally added to mRNA molecules during RNA processing. Immediately after transcription, the 3′ end of the transcript is cleaved to free a 3′ hydroxyl to which poly-A polymerase adds a chain of adenine nucleotides to the RNA in a process called polyadenylation. The poly-A tail has been extensively shown to enhance both translational efficiency and stability of mRNA (see Bernstein, P. and Ross, J., 1989, Poly (A), poly (A) binding protein and the regulation of mRNA stability, Trends Bio Sci v. 14 373-377; Guhaniyogi, J. and Brewer, G., 2001, Regulation of mRNA stability in mammalian cells, Gene, v. 265, 11-23; Dreyfus, M. and Regnier, P., 2002, The poly (A) tail of mRNAs: Bodyguard in eukaryotes, scavenger in bacteria, Cell, v. 111, 611-613).

Poly (A) tailing of in vitro transcribed mRNA can be achieved using various approaches including, but not limited to, cloning of a poly (T) tract into the DNA template or by post-transcriptional addition using Poly (A) polymerase. The first case allows in vitro transcription of mRNA with poly (A) tails of defined length, depending on the size of the poly (T) tract, but requires additional manipulation of the template. The latter case involves the enzymatic addition of a poly (A) tail to in vitro transcribed mRNA using poly (A) polymerase which catalyzes the incorporation of adenine residues onto the 3′ termini of RNA, requiring no additional manipulation of the DNA template, but results in mRNA with poly (A) tails of heterogeneous length. 5′-capping and 3′-poly (A) tailing can be performed using a variety of commercially available kits including, but not limited to Poly (A) Polymerase Tailing kit (EpiCenter), mMESSAGE mMACHINE T7 Ultra kit and Poly (A) Tailing kit (Life Technologies) as well as with commercially available reagents, various ARCA caps, Poly (A) polymerase, etc.

In addition to 5′ cap and 3′ poly adenylation, other modifications of the in vitro transcripts have been reported to provide benefits as related to efficiency of translation and stability. It is well known in the art that pathogenic DNA and RNA can be recognized by a variety of sensors within eukaryotes and trigger potent innate immune responses. The ability to discriminate between pathogenic and self DNA and RNA has been shown to be based, at least in part, on structure and nucleoside modifications since most nucleic acids from natural sources contain modified nucleosides In contrast, in vitro synthesized RNA lacks these modifications, thus rendering it immunostimulatory which in turn can inhibit effective mRNA translation as outlined above. The introduction of modified nucleosides into in vitro transcribed mRNA can be used to prevent recognition and activation of RNA sensors, thus mitigating this undesired immunostimulatory activity and enhancing translation capacity (see e.g. Kariko, K. And Weissman, D. 2007, Naturally occurring nucleoside modifications suppress the immunostimulatory activity of RNA: implication for therapeutic RNA development, Curr Opin Drug Discov Devel, v. 10 523-532; Pardi, N., Muramatsu, H., Weissman, D., Kariko, K., In vitro transcription of long RNA containing modified nucleosides in Synthetic Messenger RNA and Cell Metabolism Modulation in Methods in Molecular Biology v. 969 (Rabinovich, P. H. Ed), 2013); Kariko, K., Muramatsu, H., Welsh, F. A., Ludwig, J., Kato, H., Akira, S., Weissman, D., 2008, Incorporation of Pseudouridine Into mRNA Yields Superior Nonimmunogenic Vector With Increased Translational Capacity and Biological Stability, Mol Ther v. 16, 1833-1840. The modified nucleosides and nucleotides used in the synthesis of modified RNAs can be prepared using general methods and procedures known in the art. A large variety of nucleoside modifications are available that may be incorporated alone or in combination with other modified nucleosides to some extent into the in vitro transcribed mRNA (see, e.g., US2012/0251618). In vitro synthesis of nucleoside-modified mRNA have been reported to have reduced ability to activate immune sensors with a concomitant enhanced translational capacity.

Other components of mRNA which can be modified to provide benefit in terms of translatability and stability include the 5′ and 3′ untranslated regions (UTR). Optimization of the UTRs (favorable 5′ and 3′ UTRs can be obtained from cellular or viral RNAs), either both or independently, have been shown to increase mRNA stability and translational efficiency of in vitro transcribed mRNA (see e.g. Pardi, N., Muramatsu, H., Weissman, D., Kariko, K., In vitro transcription of long RNA containing modified nucleosides in Synthetic Messenger RNA and Cell Metabolism Modulation in Methods in Molecular Biology v. 969 (Rabinovich, P.H. Ed), 2013).

The compositions and formulations described herein may comprise an mRNA molecule (e.g., as a vaccine) for expressing a target polypeptide in a subject to induce immune response. In some embodiments, the polypeptide encoded by the mRNA molecules is an antigen from a virus, such as a coronavirus. In some embodiments, the polypeptide is an antigen from SARS-COV-2. In some embodiments, the polypeptide is the full-length or a fragment of spike protein of SARS-COV-2, or a mutant or variant thereof. For example, the polypeptide may have at least 50%, 55%, 60%, 65%, 70%, 75%, 80%, 85%, 90%, 95%, 99%, or more (and all sub-values and sub-ranges there between, including endpoints) sequence identity as the full-length or a fragment of spike protein of SARS-COV-2.

### Self-Amplifying (Self-Replicating) RNA Molecules

In some embodiments, the bioactive agents (e.g., RNA, such as samRNAs, or mRNA) packaged for delivery into or onto microneedles are replicons. A “replicon” refers to a DNA or RNA molecule that is capable of undergoing self-replication, in whole or in part, such as in a self-replicating nucleic acid molecule. In preferred embodiments, the replicon is an RNA molecule. Replicon RNA can substantially amplify the production of an encoded protein, leading to sustained translation and protein production in a target cell. In some embodiments, RNA replicons are based on or derived from viruses. A variety of suitable viruses (e.g., RNA viruses) are available, including, but not limited to, picornavirus, flavivirus, coronavirus, pestivirus, rubivirus, calcivirus, and hepacivirus. In some embodiments, the RNA replicon is derived from a virus (e.g., a coronavirus, such as SARS-COV-2). In some embodiments, replicons are positive-stranded so that they are directly translated by the host cell without requiring intermediate replication steps, such as reverse transcription. In some embodiments, replicons are derived from negative-stranded viruses. In some embodiments, the negative-stranded virus derived replicon is from Sendai virus or vesicular stomatitis virus. In some embodiments, when a positive-stranded replicon is delivered to a host cell, it is directly translated, generating an RNA-dependent RNA polymerase which then produces both antisense and sense transcripts from the delivered RNA. In some embodiments, these RNA transcripts are translated directly such that the host cell produces an encoded polypeptide, or they are further transcribed to produce more transcripts that are translated by the host cell to produce more encoded polypeptide.

In some embodiments, the RNA replicon is used as a vector to deliver a nucleic acid encoding an exogenous transcript or polypeptide to a host cell. In some embodiments, the RNA replicon contains an RNA (e.g., an mRNA) sequence that, when delivered to a host cell, results in the production of a polypeptide or active transcript. In some embodiments, the RNA sequence contains the genetic code for a selected polypeptide and the RNA replicon is said to “encode” that polypeptide or active RNA. In some embodiments, an exogenous sequence is inserted into a viral replicon by way of recombinant techniques or artificial synthesis to produce a recombinant polynucleotide comprising the exogenous sequence. In some embodiments, an exogenous polypeptide is derived from an organism other than the virus from which the viral sequence portions of the recombinant replicon are derived. In some embodiments, the replicon is engineered to encode an exogenous polypeptide such that delivery of the engineered RNA replicon into a host cell results in the production of a large amount of exogenous polypeptide by the host cell. In some embodiments, the replicon is engineered to encode a fragment or full-length of SARS-COV-2 spike protein.

A variety of methods are available for producing a recombinant replicon. In some embodiments, a recombinant replicon is generated in the laboratory by in vitro transcription (IVT) techniques. In some embodiments, IVT uses a linear DNA template, such as a cDNA, linearized bacterial plasmid, or PCR product. In some embodiments, the template DNA has a promoter sequence specific for a DNA-dependent RNA polymerase enzyme to initiate RNA synthesis. Although any suitable technique is contemplated, DNA templates are typically generated and propagated in a bacterial plasmid or are created synthetically (e.g., PCR or other synthetic DNA methods known in the art). In some embodiments, the linear DNA molecule acts as a template for an in vitro enzymatic reaction using a polymerase (e.g., a bacteriophage RNA polymerase) that results in an RNA transcript (“copy”) of the template DNA molecule. Examples of bacteriophage RNA polymerases useful in such processes include T7, T3, and SP6 RNA polymerases. In some embodiments, the template DNA comprises a viral replicon comprising four non-structural genes (e.g., nsP1-4) and a sequence encoding the exogenous polypeptide such that the mRNA transcript comprises the recombinant viral replicon encoding the viral non-structural genes (e.g., nsP1-4) and the exogenous polypeptide. Methods for producing viral replicons are available and any suitable method for producing a viral replicon is contemplated for use with the compositions and methods disclosed herein.

The disclosed recombinant viral replicons in accordance with the present disclosure have various lengths. In some embodiments, the recombinant viral replicons are about 4000, 5000, 6000, 7000, 8000, 9000, 10000, 15000, 20000, 30000, or more (and all sub-values and sub-ranges there between, including endpoints) nucleotides in length. In some embodiments, the recombinant viral replicon is 5000-25000 nucleotides in length (e.g., 8000-15000 nucleotides, or 9000-12000 nucleotides). In some embodiments, a replicon comprises a 5′ cap. In some embodiments, the 5′ cap is a 7-methylguanosine. In some embodiments, the 5′ cap enhances in vivo translation of the RNA.

In some embodiments, the 5′ nucleotide of a replicon has a 5′ triphosphate group. In some embodiments, in a capped RNA, the 5′ triphosphate group is linked to a 7-methylguanosine via a 5′-to-5′ bridge. In some embodiments, a 5′ triphosphate enhances RIG-I binding and thus promotes adjuvant effects. In some embodiments, a replicon comprises a 3′ poly-A tail. In some embodiments, the replicon includes a poly-A polymerase recognition sequence (e.g., AAUAAA) near its 3′ end. In some embodiments, a replicon for delivery to a subject is single-stranded. In some embodiments, single-stranded RNAs initiate an adjuvant effect by binding to TLR7, TLR8, RNA helicases, and/or PKR. In some embodiments, RNA is delivered in double-stranded form (dsRNA) and binds to TLR3. In some embodiments, TLR3 is triggered by dsRNA which is formed either during replication of a single-stranded RNA or within the secondary structure of a single-stranded RNA. In some embodiments, a replicon comprises (in addition to any 5′ cap structure) one or more nucleotides having a modified nucleobase. In some embodiments, a self-replicating RNA comprises one or more modified pyrimidine nucleobases, such as pseudouridine and/or 5-methylcytosine residues. In some embodiments, the RNA includes no modified nucleobases. In some embodiments, the RNA includes no modified nucleotides (e.g., all of the nucleotides in the RNA are standard A, C, G and U ribonucleotides, except for any 5′ cap structure, which, in some embodiments, comprise a 7′-methylguanosine). In some embodiments, the replicon is an RNA comprising a 5′ cap comprising a 7′-methylguanosine, and the first 1, 2, 3, or more 5′ ribonucleotides are modified at the 2′ OH position of the ribose. A variety of 2′ OH ribose modifications are available and contemplated for use with the compositions and methods disclosed herein. Examples of 2′ OH modifications include, but are not limited to: 2′-O-Me, 2′-MOE, 2′-amino, and 2′-F. In some embodiments, an RNA replicon comprises only phosphodiester linkages between nucleosides. In some embodiments, the RNA replicon comprises phosphoramidate, phosphorothioate, methylphosphonate, or other linkages (such as 2′-4′-locked/bridged sugars (e.g., LNA, ENA, or UNA)).

In some embodiments, the polypeptide encoded by the recombinant viral replicon is an antigen from a virus, such as a coronavirus. In some embodiments, the polypeptide encoded by the recombinant viral replicon is an antigen from SARS-COV-2. In some embodiments, the polypeptide encoded by the recombinant viral replicon is the full-length or a fragment of spike protein of SARS-COV-2, or a mutant or variant thereof. For example, the polypeptide may have at least 50%, 55%, 60%, 65%, 70%, 75%, 80%, 85%, 90%, 95%, 99%, or more (and all sub-values and sub-ranges there between, including endpoints) sequence identity as the full-length or a fragment of spike protein of SARS-COV-2.

### Other DNA or RNA Molecules

In addition to mRNA, other nucleic acid payloads may be used for this invention. In some embodiments, the nucleic acid in the compositions and formulations described herein may comprise a DNA or RNA molecule other than mRNAs and samRNAs, such as antisense oligonucleotide, small interfering RNA (siRNA), short hairpin RNAs or small hairpin RNA (shRNA), plasmid DNA, microRNA (miRNA), miRNA inhibitors (antagomirs/antimirs), messenger-RNA-interfering complementary RNA (micRNA), multivalent RNA, dicer substrate RNA, complementary DNA (cDNA), etc. In some embodiments, the nucleic acid in the compositions and formulations described herein comprises an siRNA and/or shRNA.

For oligonucleotides, methods of preparation include but are not limited to chemical synthesis and enzymatic, chemical cleavage of a longer precursor, in vitro transcription as described above, etc. Methods of synthesizing DNA and RNA nucleotides are widely used and well known in the art (see, e.g. Gait, M. J. (ed.) Oligonucleotide synthesis: a practical approach, Oxford [Oxfordshire], Washington, D.C.: IRL Press, 1984; and Herdewijn, P. (ed.) Oligonucleotide synthesis: methods and applications, Methods in Molecular Biology, v. 288 (Clifton, N.J.) Totowa, N.J.: Humana Press, 2005; both of which are incorporated herein by reference).

For plasmid DNA, preparation for use with this invention commonly utilizes but is not limited to expansion and isolation of the plasmid DNA in vitro in a liquid culture of bacteria containing the plasmid of interest. The presence of a gene in the plasmid of interest that encodes resistance to a particular antibiotic (penicillin, kanamycin, etc.) allows those bacteria containing the plasmid of interest to selectively grow in antibiotic-containing cultures. Methods of isolating plasmid DNA are widely used and well known in the art (see, e.g. Heilig, J., Elbing, K. L. and Brent, R (2001) Large-Scale Preparation of Plasmid DNA. Current Protocols in Molecular Biology. 41: II: 1.7:1.7.1-1.7.16; Rozkov, A., Larsson, B., Gillstrom, S., Bjornestedt, R. and Schmidt, S. R. (2008), Large-scale production of endotoxin-free plasmids for transient expression in mammalian cell culture. Biotechnol. Bioeng., 99:557-566; and U.S. Pat. No. 6,197,553B1). Plasmid isolation can be performed using a variety of commercially available kits including, but not limited to Plasmid Plus (Qiagen), GenJET plasmid MaxiPrep (Thermo) and PureYield MaxiPrep (Promega) kits as well as with commercially available reagents.

### Encoded Polypeptides

The nucleic acids delivered by the LNPs as described herein can encode polypeptides, which can be produced after entering cells. Alternatively, either the delivered nucleic acids or the polypeptides encodable by the delivered nucleic acids may perform the designed biological function in cells. Generally there is no limitation on the type or identity of the encoded polypeptides. For example, the delivered nucleic acids may encode a polypeptide capable of inducing host immune response. Such polypeptide may originate from a pathogen (such as a viral protein) or may comprise a therapeutic drug (e.g., a therapeutic protein or antibody). The nucleic acid-LNP delivery system described herein may be used for, e.g., prophylactic vaccines, therapeutic vaccines, therapeutic delivery, etc. For example, prophylactic vaccines may be prepared used the nucleic acid-LNP delivery system described herein targeting COVID-19, respiratory syncytial viruses, influenza virus, etc., in which a viral protein may be encodable by the delivered nucleic acid to illicit host immune response. Therapeutic vaccines may be prepared used the nucleic acid-LNP system described herein to deliver at least one target antigens (e.g., tumor antigens), such as mRNA-5671 developed by Moderna/Merck & Co. for KRAS-mutated cancers and BNT113 developed by BioNTech for HPV-16+ cancers, both of which are in phase I. Multi-antigen vaccines may be used as personalized cancer vaccines (PCVs), such as Moderna's mRNA-4157 as a monotherapy and in combination with pembrolizumab.

The nucleic acid-LNP delivery system described herein may be used for delivering therapeutics, too. For example, therapeutic proteins such as erythropoietin (EPO), and monoclonal antibodies may be encoded by the delivered nucleic acids. Such encoded monoclonal antibodies may modulate host immune responses, such as directly antagonizing pathogens (e.g., viruses, tumors or cancers, etc.) or boosting immune system against pathogens. For example, an mRNA delivered using the system described herein may encode a bispecific antibody that binds to CD3 on T cells and a target antigen on tumor cells. For genetic disorders and rare diseases, mRNA therapeutics will probably need to compete with gene therapies. For a summary of current progress on mRNA platforms, see Xie et al., Nat Rev Drug Discov. 2021; 20 (10): 735-736, the contents of which are incorporated by reference herein to its entirety.

In some embodiments, the nucleic acid-LNP system described herein may be used to deliver mRNAs encoding therapeutic antibodies, either modified or unmodified. The benefit of such system may include, e.g., endogenous protein synthesis benefiting from native post-translation modifications and a simplified manufacturing method that does not require cell culture and extensive purification and characterization of the protein product. An example are Acuitas LNPs, using purified nucleoside-modified mRNAs encoding the light and heavy chains of VRC01, a broadly neutralizing antibody against HIV-1. The feasibility of therapeutic non-modified mRNA-encoded antibodies was confirmed in a study by CureVac, also using Acuitas LNPs, where an IgG mAb with broad neutralization ability for a variety of rabies strains was chosen, as well as a heavy chain-only Vh domain-based (VHH) neutralizing agent against the botulinum toxin. An mRNA-encoded rituximab, targeting CD20, the gold standard for treating non-Hodgkin's lymphoma, was also produced. Bispecific antibodies that recruit T cells to tumor cells were also encoded in modified mRNA constructs and delivered in vivo using a commercial transfection reagent, TransIT, which is not as efficient as current LNPs for liver delivery. Bispecific antibodies in the VHH format, such as one VHH that binds the conserved influenza A matrix protein 2 ectodomain (M2e) was genetically linked to a second VHH that specifically binds to the mouse Fc receptor IV (Fc RIV) in order to recruit innate immune cells expressing Fc RIV to influenza infected cells expressing M2e. These examples of nucleoside-modified mRNA constructs may be delivered using DOTAP/cholesterol LNPs by intratracheal instillation into the lung.

Other examples on assembly and structure of lipid nanoparticles, as well as determinants of performance of the mRNA delivery system for vaccines, may be found in Buschmann et al., Vaccines 2021, 9:65, the contents of which is incorporated by reference herein to its entirety.

### Lipid Nanoparticles (LNPS)

As noted above, the nucleic acids are formulated with lipid nanoparticles. The term “lipid nanoparticle” or “LNP” refers to particles having at least one dimension on the order of nanometers (e.g., 1-1,000 nm) which include one or more of the compounds of structure (I) or other specified cationic lipids. In some embodiments, lipid nanoparticles are included in a formulation that can be used to deliver an active agent or therapeutic agent, such as a nucleic acid (e.g., mRNA) to a target site of interest (e.g., cell, tissue, organ, tumor, and the like). In some embodiments, the lipid nanoparticles of the invention comprise a nucleic acid. Such lipid nanoparticles typically comprise a compound of structure (I) and one or more excipient selected from neutral lipids, charged lipids, steroids and polymer conjugated lipids. In some embodiments, the active agent or therapeutic agent, such as a nucleic acid, may be encapsulated in the lipid portion of the lipid nanoparticle or an aqueous space enveloped by some or all of the lipid portion of the lipid nanoparticle, thereby protecting it from enzymatic degradation or other undesirable effects induced by the mechanisms of the host organism or cells e.g. an adverse immune response.

In various embodiments, the lipid nanoparticles have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm, and are substantially non-toxic. In certain embodiments, nucleic acids, when present in the lipid nanoparticles, are resistant in aqueous solution to degradation with a nuclease. Lipid nanoparticles comprising nucleic acids and their method of preparation are disclosed in, e.g., U.S. Patent Publication Nos. 2004/0142025, 2007/0042031 and PCT Pub. Nos. WO 2013/016058 and WO 2013/086373, the full disclosures of which are herein incorporated by reference in their entirety for all purposes.

As used herein, “lipid encapsulated” refers to a lipid nanoparticle that provides an active agent or therapeutic agent, such as a nucleic acid (e.g., mRNA), with full encapsulation, partial encapsulation, or both. In an embodiment, the nucleic acid (e.g., mRNA) is fully encapsulated in the lipid nanoparticle.

In some embodiments, the use of dendrimers can be expressly excluded.

As used herein, the term “aqueous solution” refers to a composition comprising water.

In some embodiments, LNPs used for formulating viral replicons described herein do not include dendrimer nanoparticles. For example, examples of LNPs may comprise nanoparticles which do not comprise dendrimers.

Examples of LNPs used for formulating viral replicons include at least one structural lipid and a functional lipid (e.g., an ionizable lipid). Cholesterol, DSPE, and 1,2-distearoyl-sn-glycero-3-phosphocholine (DSPC) are examples of structural lipids that can be utilized. For example, DSPC may help to form a stable bilayer underneath the PEG surface and cholesterol plays several roles, including filling gaps in the particle, limiting LNP-protein interactions and possibly promoting membrane fusion. Other examples, include phosphatidylethanolamines, phosphatidylcholines, phosphatidylserines, and phosphatidylglycerols, dioleoyl trimethylammonium propane (DOTAP), 1,2-distearyloxy-N,N-dimethyl-3-aminopropane (DSDMA), 1,2-dioleyloxy-N,Ndimethyl-3-aminopropane (DODMA), 1,2-dilinoleyloxy-N,N-dimethyl-3-aminopropane (DLinDMA), 1,2-dilinolenyloxy-N,N-dimethyl-3-aminopropane (DLenDMA), acyl zwitterionic lipids, ether zwitterionic lipids, DPPC, DOPC, dodecylphosphocholine, etc.

Examples of ionizable lipids are known in the art, such as MC3, lipid 319, 5A2-SC8, 306Oi10, DLin-MC3-DMA, C12-200, ALC-0315, A9, SM-102, etc. Ionizable lipids may play a central role by being neutral at physiological pH, thus eliminating any cationic charge in circulation, but becoming protonated in the endosome at pH ˜6.5 to facilitate endosomal release. Other examples of ionizable lipids may include Moderna Lipid 5, Arcturus Lipid 2,2 (8,8) 4C CH3, and Genevant CL1. Some of these ionizable lipids have the structure and theoretical pKa values as below:

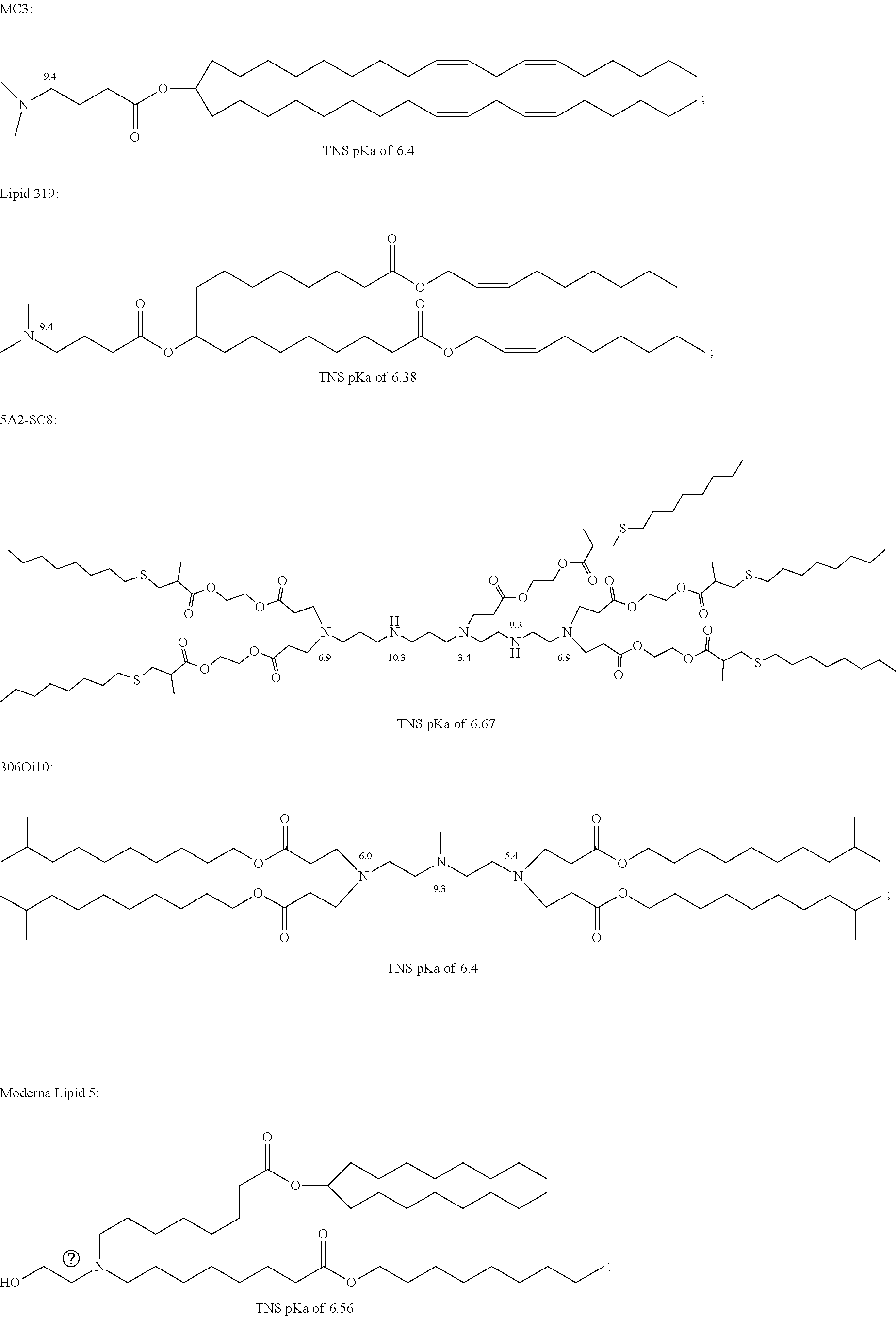

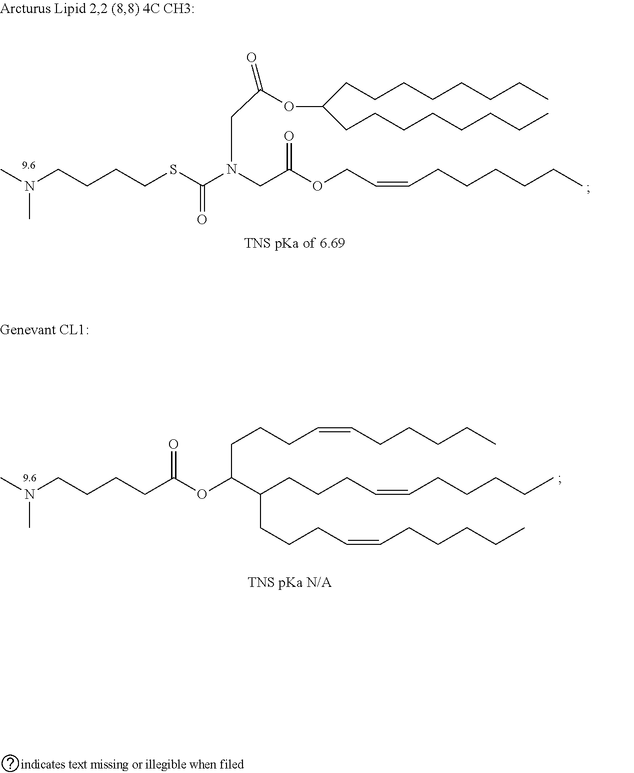

For other examples of useful lipids, see Buschmann et al., Vaccines 2021, 9:65, the contents of which is incorporated by reference herein to its entirety.

In some embodiments, the recombinant viral replicon is formulated using a microfluidic mixing device. Microfluidic mixing devices facilitate controlled, bottom-up, molecular self-assembly of nanoparticles via custom-engineered microfluidic mixing cartridges that allow millisecond mixing of nanoparticle components at the nanoliter scale.

Rapid mixing on a small scale allows reproducible control over particle formation compared to traditional methods of nanoparticle formation (such as hand mixing). In some embodiments, the microfluidic device is a NanoAssemblr™. (Precision NanoSystems).

### Encapsulation in Liposomes

In some embodiments, the one or more bioactive agents (e.g., polypeptides, DNA, RNA or recombinant viral replicons) are encapsulated in a liposome. In some embodiments, various amphiphilic lipids form bilayers in an aqueous environment to encapsulate a bioactive agent-containing aqueous core as a liposome. In some embodiments, these lipids have an anionic, cationic or zwitterionic hydrophilic head group. In some embodiments, some phospholipids are anionic whereas others are zwitterionic. Suitable classes of phospholipid include, but are not limited to, phosphatidylethanolamines, phosphatidylcholines, phosphatidylserines, and phosphatidylglycerols. Examples of cationic lipids include, but are not limited to, dioleoyl trimethylammonium propane (DOTAP), 1,2-distearyloxy-N,N-dimethyl-3-aminopropane (DSDMA), 1,2-dioleyloxy-N,N-dimethyl-3-aminopropane (DODMA), 1,2-dilinoleyloxy-N,N-dimethyl-3-aminopropane (DLinDMA), 1,2-dilinolenyloxy-N,N-dimethyl-3-aminopropane (DLenDMA). Zwitterionic lipids include, but are not limited to, acyl zwitterionic lipids and ether zwitterionic lipids. Examples of useful zwitterionic lipids are DPPC, DOPC and dodecylphosphocholine. Other examples of useful cationic polymers may include example poly (L-lysine), polyethylenimine (PEI), DEAE-dextran, poly (β-amino esters) (PBAE), hyperbranched poly (β-amino esters) (hPBAEs), poly (amine-co-ester) (PACE) terpolymer, disulfide-linked poly (amido amine) (pABOL), and chitosan. In some embodiments, the lipids are saturated. In some embodiments, the lipids are unsaturated.

In some embodiments, liposomes are formed from a single lipid or from a mixture of lipids. In some embodiments, a mixture comprises: (i) a mixture of anionic lipids; (ii) a mixture of cationic lipids; (iii) a mixture of zwitterionic lipids; (iv) a mixture of anionic lipids and cationic lipids; (v) a mixture of anionic lipids and zwitterionic lipids; (vi) a mixture of zwitterionic lipids and cationic lipids; or (vii) a mixture of anionic lipids, cationic lipids and zwitterionic lipids. In some embodiments, a mixture comprises both saturated and unsaturated lipids. In some embodiments, a mixture comprises DSPC (zwitterionic, saturated), DlinDMA (cationic, unsaturated), and/or DMPG (anionic, saturated). In some embodiments, where a mixture of lipids is used, not all of the component lipids in the mixture need to be amphiphilic, e.g., one or more amphiphilic lipids are mixed with cholesterol.

In some embodiments, the hydrophilic portion of a lipid is modified by attachment (e.g., covalent attachment) of a polyethylene glycol (also referred to as PEGylation). In some embodiments, PEGylation increases stability and prevent non-specific adsorption of the liposomes. In some embodiments, lipids are conjugated to PEG using any suitable technique.

Liposomes are usually divided into three groups: multilamellar vesicles (MLV); small unilamellar vesicles (SUV); and large unilamellar vesicles (LUV). MLVs have multiple bilayers in each vesicle, forming several separate aqueous compartments. SUVs and LUVs have a single bilayer encapsulating an aqueous core; SUVs typically have a diameter <50 nm, and LUVs have a diameter >50 nm. In some embodiments, liposomes are LUVs with a diameter in the range of 50-220 nm. In some embodiments, for a composition comprising a population of LUVs with different diameters: (i) at least 80% by number have diameters in the range of 20-220 nm; (ii) the average diameter (Zav, by intensity) of the population are in the range of 40-200 nm; and/or (iii) the diameters have a polydispersity index <0.2. A variety of techniques for preparing suitable liposomes are available and any suitable liposomal preparation technique is contemplated.

In some embodiments, encapsulating a bioactive agent increases the stability of the bioactive agent. In some embodiments, the time that a unit dose of a microneedle composition comprising a recombinant viral particle encoding an antigen remains effective in inducing a detectable immune response to the antigen is increased by encapsulating the replicon in liposomes, relative to the replicon without encapsulation. In some embodiments, the increase is about or more than about 10%, 25%, 50%, 75%, 100%, 200%, 500%, or more (and all sub-values and sub-ranges there between, including endpoints). In some embodiments, the stability of the bioactive agent is more than doubled by encapsulating in liposomes. In some embodiments, encapsulating a bioactive agent in a liposome is effective in increasing efficiency of delivering the bioactive agent to a cell. In some embodiments, encapsulating in liposomes decrease the amount of bioactive agent necessary to achieve a desired result (e.g., a protective immune response) by 10%, 20%, 30%, 40%, 50%, 60%, 70%, 80%, 90%, 95%, or more (and all sub-values and sub-ranges there between, including endpoints), as compared to the amount required if the bioactive agent were delivered without encapsulation.

### Dehydrated Compositions

Disclosed herein, in some embodiments, are microneedle devices for administering a recombinant viral replicon or RNA molecule encoding an exogenous polypeptide comprising: a substrate comprising a plurality of microneedles; and a composition comprising a recombinant viral replicon or RNA molecule encoding an exogenous polypeptide coated onto or embedded into the plurality of microneedles. Examples of devices can be found in, e.g., U.S. Pat. Nos. 11,040,112, 10,723,692, 10,221,127, 10,166,298, 10,106,490, 9,738,593, 9,737,619, 9,675,668, 9,447,164, 9,301,993, 9,295,689, 8,754,062, 10,022,436 and 10,363,303 and PCT Application Nos. PCT/US2017/045161 and PCT/US2020/044196, each of which is incorporated by reference herein to its entirety. Also disclosed herein, in some embodiments, are methods of preparing a microneedle device, comprising: obtaining a substrate comprising a plurality of microneedles; and coating or embedding a recombinant viral replicon encoding an exogenous polypeptide onto or into the plurality of microneedles. Also disclosed herein, in some embodiments, are methods of inducing an immune response in an individual in need thereof, comprising: (a) contacting the dermal surface of an individual with a microneedle device comprising (i) a plurality of microneedles comprising a recombinant viral replicon encoding an exogenous polypeptide coated onto or embedded into the plurality of microneedles, and (b) delivering the recombinant viral replicon to the individual, thereby inducing an immune response in the individual.

In some embodiments, the RNA compositions described herein are provided in a dehydrated form. In some embodiments, a recombinant viral replicon is in a dehydrated form, such as before or after applying to or embedding within microneedles. In some embodiments, the replicon encapsulated in a liposome is in a dehydrated form. In some embodiments, dehydration offers several advantages including increased stability of the composition, increased shelf-life of the composition, and reduced weight of the composition.

In general, the term “dehydration” refers to the removal of an amount of water from a composition. In some embodiments, a composition is dehydrated so as to remove about or at least about 10%, 20%, 30%, 40%, 50%, 60%, 70%, 80%, 90%, 95% or more (and all sub-values and sub-ranges there between, including endpoints) of a starting amount of water. In some embodiments, at least 50% of a starting amount of water is removed. In some embodiments, the amount of water desired to be removed depends on the starting amount of water, so as to arrive at an amount of water at or below a target amount. In some embodiments, a composition is dehydrated so as to contain about or less than about 10%, 9%, 8%, 7%, 6%, 5%, 4%, 3%, 2%, 1%, 0.1%, 0.01%, or less water. In some embodiments, the dehydrated form contains less than 1% water. In some embodiments, a composition, or component thereof, is dehydrated to remove substantially all, e.g., 90%, 91%, 92%, 93%, 94%, 95%, 96%, 97%, 98%, 98.5%, 99%, or more (and all sub-values and sub-ranges there between, including endpoints) water content. In some embodiments, a dehydrated replicon composition is substantially free of water content. In some embodiments, 98% of the water content of a composition is removed. In some embodiments, the starting form of a composition to be dehydrated is liquid, semi-liquid, semi-solid, solid, or a gel. In general, a composition that has been dehydrated is referred to as a “dried” composition. In some embodiments, a dried composition is in a powdered form. In some embodiments, a dried composition is gel-like. In some embodiments, a dehydrated composition includes a liquid replicon composition that has been reduced to a dried composition. In some embodiments, a dehydrated replicon composition has increased stability at room temperatures (e.g., temperatures between 20° C. to 30° C., or 21° C. to 28° C.). In some embodiments, a dehydrated replicon composition has increased shelf-life at room temperatures. In some embodiments, the increase in stability at room temperature is about or more than about 10%, 25%, 50%, 75%, 100%, 200%, 500%, or more, (and all sub-values and sub-ranges there between, including endpoints) relative to the composition before dehydration, or an equivalent composition provided in liquid form.

In some embodiments, dehydration is accomplished by any number of methods. In one embodiments, dehydration of the replicon composition is accomplished by freeze-drying, also referred to as “lyophilization.” In some embodiments, lyophilization involves the steps of freezing the water in a composition to the solid state, followed by sublimation. In some embodiments, sublimation is performed in a vacuum. In some embodiments, heat is applied to the composition to accelerate the sublimation process. In some embodiments, lyophilization removes substantially all of the water content of the composition. In some embodiments, lyophilization is used when low-temperature drying methods are desired. In some embodiments, freeze-drying machines are readily available from a number of different manufacturers.

In some embodiments, dehydration methods for compositions and formulations described herein do not include lyophilization. In some embodiments, a replicon composition is air-dried. In some embodiments, the replicons are washed with a volatile alcohol (e.g., isopropanol or ethanol). In some embodiments, this step replaces the water content in the composition with the volatile alcohol and precipitates the nucleic acid molecule. In some embodiments, after a step of centrifugation to pellet the precipitated nucleic acid molecule, the volatile alcohol is removed, such as by pipetting or pouring off the volatile alcohol, and the pelleted nucleic acid molecule is allowed to air-dry. In some embodiments, dehydration comprises the use of a desiccant. Examples of desiccants include alumina, aluminum amalgam, barium oxide, barium perchlorate, boric anhydride, calcium chloride (anhydrous), calcium oxide, calcium sulphate (anhydrous), copper (II) sulfate (anhydrous), magnesium amalgam, magnesium perchlorate (anhydrous), magnesium sulphate (anhydrous), phosphorus pentoxide, potassium, potassium carbonate (anhydrous), potassium hydroxide, silica gel, sodium, sodium hydroxide, sodium-potassium alloy, sodium sulphate (anhydrous), sulfuric acid, and the like. Other available methods of dehydration include heat drying, freeze-drying with liquid nitrogen, and spray drying.

In some embodiments, at least a part of water content in the compositions and/or formulations described herein is removed by the drying methods described herein. In some embodiments, such drying methods do not include spray-freeze drying. In some embodiments, such drying methods do not include lyophilization. In some embodiments, such drying methods remove certain amount, but not all, of water contents from the compositions and formulations described herein for vitrification (e.g., into sugar glass). In some embodiments, the compositions and formulations described herein are partly dried prior to vitrification. The drying may be used to reduce the volume of the compositions and formulations, and, thus, increase the concentration of the LNP in the compositions and formulations. Optionally, the drying process may be used to adjust the concentration of the water content and/or the LNP concentration in the compositions and formulations prior to vitrification. Optionally, the compositions and formulations may be added with water and/or other solutions to adjust the water content and/or the LNP concentration in the compositions and formulations.

In some embodiments, dehydrated bioactive agents are coated directly onto microneedle structures for administration to a subject. In some embodiments, a liquid replicon composition is spray-dried directly onto the microneedle to coat the structure. In another example, the microneedle is dipped into the liquid replicon composition and then the composition is air-dried onto the structure. In some embodiments, the liquid replicon or polypeptide composition is coated onto the microneedle using a microfluidic device (e.g., the BioDot printer described herein). In some embodiments, a dehydrated replicon composition is coated directly onto a metal microneedle structure. In other embodiments, the dehydrated replicon composition is coated directly onto a polymer microneedle structure. In yet other embodiments, the dehydrated replicon composition is coated directly onto a polymer-coated microneedle structure. In some embodiments, the composition including the microneedles themselves is dehydrated.

In some embodiments, the recombinant viral replicon is packaged onto a microneedle using a microfluidic dispensing device. Microfluidic dispensing devices are non-contact liquid handling systems with high speed aspirating and dispensing capabilities that reproducibly dispense an accurate printing volume onto a microneedle, plurality of microneedles, or microneedle array. A microfluidic dispensing device typically utilizes a moveable stage holding a ceramic needle which accurately picks up a pre-determined volume of reagent (e.g., replicon RNA) and then ejects (prints) nanoliter volumes onto a substrate (e.g., a microneedle array) positioned on a printing table. In some embodiments, the microfluidic dispensing device is a BioDot AD1520 tabletop workstation.

In some embodiments, a dehydrated bioactive agent (e.g., a polypeptide or replicon) is incorporated into the microneedle itself (e.g., embedded into the microneedle). In some embodiments, a dehydrated replicon is mixed with a polymer before molding and polymerization. In some embodiments, the replicon-polymer composition is then molded and polymerized to form a solid microneedle structure wherein the replicon is contained within the microneedle structure. In some embodiments, the polymer substance is dissolvable, biodegradable, biosoluble, or a combination thereof such that upon application of the microneedle to the skin of a subject, the polymer is dissolved, biodegraded, and/or solubilized and the replicon is released.

### Vitrification and Sugar Glass

LNPs or vaccines described herein may be dried and/or vitrified into sugar glass for storage and/or transportation. In some embodiments, such vitrification into sugar glass increases stability of the LNPs or vaccines described herein, especially at a temperature or environment different from the traditional low temperature environment to store/transport RNAs, LNPs, and/or vaccines. For example, such dried/vitrified LNPs or vaccines, in sugar glass status, may be more stable at room temperature (e.g., from about 20° C. to about 30° C.), compared to other LNPs or vaccines not dried and/or vitrified.

Examples of vitrification processes known in the art may be used in the present disclosure. See Fahy and Wowk (2015) Principles of cryopreservation by vitrification. Methods Mol Biol. 1257:21-82. Also see PCT Application no. PCT/US2017/045161, incorporated herein by reference to its entirety. Vitrification is an alternative to slow freezing for the cryopreservation of cells or other compounds or compositions, such as LNPs or vaccines described herein. Compared to the slow-freezing method, a much faster freezing rate are required.

In some embodiments, the substance such as LNPs or vaccines described herein is formulated to be vitrified into a sugar glass structure. For a review of sugar glass, see Santivarangkna et al. (2008) Protection mechanisms of sugars during different stages of preparation process of dried lactic acid starter cultures. Food Microbiol. 25 (3): 429-41 (incorporated herein by reference in its entirety, including for all methods, materials, etc.). In some embodiments, the substance loaded into the microtip reservoirs of a Micro Array are formulated as a sugar glass or sugar crystal. In some embodiments, the sugar glass comprises sucrose, glucose, galactose, fructose, trehalose, maltose, or a combination thereof. In some embodiments, the sugar glass comprises a disaccharide. In some embodiments, the sugar glass comprises a mixture or combination of different sugar components in suitable ratios. Any suitable sugar, including any described herein or otherwise known, can be utilized in any suitable amount. For example, in some embodiments, the sugar glass is a sucrose and/or trehalose sugar glass. In some embodiments, the sugar glass comprises trehalose. Other examples of sugar useful in the compositions and formulations described herein to form a sugar glass may comprise d-glucose, d (−)-fructose, d-sorbitol, sucrose, d (+)-trehalose, and raffinose. In some embodiments, any sugar type suitable for vitrification and sugar glass formation may be used for the compositions and formulations described herein. Examples of formations and properties of sugar glasses include those known in the art. Two examples include forming arrays of projections such as seen with the Vaxipatch technology, or vitrifying the material into a sugar glass that is in a vial, an ampoule, syringe, etc., which can be reconstituted and administered.

In some embodiments, a forming press bends the plurality of microtips out of planarity to an angle relative to the plane of the substrate. In some embodiments, the forming press comprises a plurality of forming supports and a plurality of forming dies. In some embodiments, each forming die in the plurality of forming dies comprises a plurality of projections that bend the microtips out of planarity to an angle relative to the plane of the substrate. In some embodiments, each forming support in the plurality of forming supports comprises a plurality of microtip. In some embodiments, a vaccine is trapped in a sugar glass. In contrast to conventional vaccines in suspension, vaccines immobilized within a sugar glass are able to withstand degradation at relatively high temperatures for extended periods of time (e.g. months). The ability to preserve vaccines without the need for refrigeration (i.e. cold chain for storage and transport) is of great importance in third world countries and/or areas in which electricity and/or refrigeration vehicles are not readily available.

The term “sugar glass” in the present disclosure may refer to a solid or semi-solid structure formed by vitrification of a formulation or composition containing LNPs described herein. In that sense, a sugar glass, as a whole structure or product, includes a formulation or composition containing LNPs described herein, plus the solution (e.g. drying buffers) used to dry and/or vitrify the LNP formulation or composition. In some embodiments, when the term “sugar glass” is used in parallel to the LNP formulation or composition, it may refer to only the vitrified solution (as a “container”) which does not include the LNP formulation or composition. In some embodiments lyophilization is expressly excluded from the methods of forming the sugar glass materials.

The formulation of LNPs or vaccines described herein may comprise a sugar, such as a disaccharide (e.g., sucrose, trehalose, or others described herein or a combination thereof). For example, the concentration of the sugar in total ranges between 0 w/w and about 60% w/w (or any sub value or sub range there between, inclusive of endpoints) prior to drying steps. For example, the concentration of the sugar ranges between 0 w/w and about 60% w/w (e.g., about 0-30% w/w, 0-25% w/w, 0-20% w/w, 0-15% w/w, 0-10% w/w, about 5% w/w, about 8% w/w, about 10% w/w, about 15% w/w, about 20% w/w, about 25% w/w, about 30% w/w, about 35% w/w, about 40% w/w, about 45% w/w, about 50% w/w, about 55% w/w, about 60% w/w, or more, and all sub-values and sub-ranges there between, including endpoints) prior to drying. Examples of sugar concentrations may be found in US Application No. US20200069599A1, incorporated by reference herein in its entirety.

Sugar glass in the present disclosure may contain water (e.g., moisture). In some embodiments, the water/moisture content in the sugar glass may be important for the property of the sugar glass. Moisture content can be measured by various methods (e.g., by weight) before and/or after drying. Other measurement methods, such as head space laser measurement, may also be used. Higher moisture contents may help preserve the RNA activity after reconstitution. However, higher moisture contents may also result in a lower glass transition temperature Tg. Tg may also be important for the property of the sugar glass. Tg can be measured by various methods, such as differential scanning calorimetry. Moisture content and Tg can be considered and regulated in a balance to yield a proper sugar glass containing the samRNA-containing LNPs described herein, which may exhibit a commercially viable amount of room temperature stability (e.g., about 1, 2, 3, or 4 weeks). In some embodiments, the moisture content of the vitrified or sugar glass compositions may be, for example, from about 5% to about 95%, from about 20% to about 95%, from about 40% to about 80%, from about 50% to about 75%, or any sub-range or sub-value within those examples of ranges. In some embodiments the water/moisture content of the vitrified or sugar glass compositions may be, for example, about 5%, 10%, 15%, 20%, 25%, 30%, 35%, 40%, 45%, 50%, 55%, 60%, 65%, 70%, 75%, 80%, 85%, 90%, 95%, or more (and all sub-values and sub-ranges there between, including endpoints). In some embodiments the water/moisture content of the vitrified or sugar glass compositions may be, for example, about 10% to about 50%, about 15% to about 40%, about 20% to about 35%, about 20% to about 30%, about 23% to about 27%, and all sub-values and sub-ranges there between, including endpoints. The water content can be modified by as desired, for example, depending on various LNP compositions or formulations, as well as various Tg used.

### Microneedles and Microneedle Arrays

Disclosed herein, in some embodiments, are microneedle devices for administering a recombinant viral replicon or RNA molecule encoding an exogenous polypeptide comprising: a substrate comprising a plurality of microneedles; and a composition comprising a recombinant viral replicon or RNA molecule encoding an exogenous polypeptide coated onto or embedded into the plurality of microneedles. Also disclosed herein, in some embodiments, are methods of preparing a microneedle device, comprising: obtaining a substrate comprising a plurality of microneedles; and coating or embedding a recombinant viral replicon encoding an exogenous polypeptide onto or into the plurality of microneedles. Also disclosed herein, in some embodiments, are methods of inducing an immune response in an individual in need thereof, comprising: (a) contacting the dermal surface of an individual with a microneedle device comprising (i) a plurality of microneedles comprising a recombinant viral replicon encoding an exogenous polypeptide coated onto or embedded into the plurality of microneedles, and (b) delivering the recombinant viral replicon to the individual, thereby inducing an immune response in the individual. Examples of microneedles, formable into microarrays, can be found in, e.g., U.S. Pat. No. 10,022,436 and PCT Application no. PCT/US2017/045161, each of which is incorporated herein by reference to its entirety.

Microneedles are structures of typically micrometer to millimeter size, and preferably designed to pierce the skin and deliver a composition to the epidermis or dermis of a subject. Microneedles offer some advantages over traditional sub-cutaneous or intramuscular injections. In some embodiments, microneedles are used to deliver the replicon directly to the immune cells in the skin, which is advantageous for immunization purposes. The amount of replicon needed for microneedle administration, compared to traditional sub-cutaneous or intramuscular injections, is smaller and can reduce production cost and time. In some embodiments, the microneedle is self-administered. In some embodiments, the replicon is dried onto the microneedle, which greatly increases the stability of the composition at room temperature. Microneedle administration is painless, making it a more tolerated form of administration.

“Microneedles” (also known as microtips) provide an alternative to conventional transdermal patches and syringe injections. Microneedles penetrate skin only to a depth of about 400 to 500μπΘ, an insufficient depth to reach nerves or blood vessels. Additionally, penetrating agents are not typically used with microneedles, and thus microneedles are not transdermal delivery devices per se. Microneedles are able to penetrate the stratum corneum and the epidermis, but penetrate into only a portion of the dermis. Without full penetration of the dermis, microneedles are more correctly referred to as “transepithelial delivery devices” rather than “transdermal delivery devices.” Microneedles arranged in a small array (also known as a microneedle array, hereinafter referred to as a “MicroArray”), each coated with a very small amount of a drug or other substance, provide ease of self-administration, reduced drug delivery costs, avoidance of hypodermic needle stick injuries, smaller dosage volumes, less fear compared to a home injection when self-administering, and reduced healthcare training burden. Microneedles are painless because, as mentioned, the individual needles are too short in length to stimulate nerve endings. Examples of microneedles useful in the present disclosure may be found in, e.g., U.S. Pat. Nos. 11,040,112, 10,723,692, 10,221,127, 10,166,298, 10,106,490, 9,738,593, 9,737,619, 9,675,668, 9,447,164, 9,301,993, 9,295,689, 8,754,062, 10,022,436 and 10,363,303 and PCT Application Nos. PCT/US2017/045161 and PCT/US2020/044196, each of which is incorporated by reference herein to its entirety.

In some embodiments, microneedles are solid structures. In some embodiments, microneedles are hollow structures. In some embodiments, bioactive agents for delivery (e.g., polypeptides or recombinant viral replicons or RNA molecules) are released through hollow structures (e.g., a liquid composition is injected or infused into the skin). In some embodiments, bioactive agents (e.g., polypeptides or recombinant viral replicons or RNA molecules) are packaged onto a microneedle (for example, coated onto a surface of the microneedle after formation). In some embodiments, the bioactive agents are packaged onto a microneedle as a dried form. In some embodiments, the bioactive agents are dehydrated after being packaged onto a microneedle. In some embodiments, compositions are packaged into a microneedle (for example, forming part of the microneedle itself, such as by deposition into the interior of the microneedle, or by inclusion in a mixture used to form the microneedle). In some embodiments, the replicon is dissolved in the skin compartment. In some embodiments, the replicon is injected into the skin. In some embodiments, microneedles are formed in an array comprising a plurality of microneedles. In some embodiments, the microneedle array is a 5×5 array of microneedles. In some embodiments, the microneedle array is physically or operably coupled to a solid support or substrate. In some embodiments, the solid support is a patch. In some embodiments, the microneedle array is applied directly to the skin for intradermal administration of a composition.

A microneedle array patch can be any suitable shape or size. In some embodiments, a microneedle array patch is shaped to mimic facial features, e.g., an eyebrow. In some embodiments, a microneedle array patch is the smallest size allowable to deliver a selected amount of bioactive agent.

The size and shape of the microneedles varies as desired. In some embodiments, microneedles include a cylindrical portion physically or operably coupled to a conical portion having a tip. In some embodiments, microneedles have an overall pyramidal shape or an overall conical shape. In some embodiments, the microneedle includes a base and a tip. In some embodiments, the tip has a radius that is less than or equal to about 1 micrometer. In some embodiments, the microneedles are of a length sufficient to penetrate the stratum corneum and pass into the epidermis or dermis. In certain embodiments, the microneedles have a length (from their tip to their base) between about 0.1 micrometer and about 5 millimeters in length, for instance about 5 millimeters or less, 4 millimeters or less, between about 1 millimeter and about 4 millimeters, between about 500 micrometers and about 1 millimeter, between about 10 micrometers and about 500 micrometers, between about 30 micrometers and about 200 micrometers, or between about 250 micrometers to about 1,500 micrometers. In some embodiments, the microneedles have a length (from their tip to their base) between about 400 micrometers to about 600 micrometers.

In some embodiments, the size of individual microneedles is optimized depending upon the desired targeting depth or the strength requirements of the needle to avoid breakage in a particular tissue type. In some embodiments, the cross-sectional dimension of a transdermal microneedle is between about 10 nm and 1 mm, or between about 1 micrometer and about 200 micrometers, or between about 10 micrometers and about 100 micrometers. In some embodiments, the outer diameter of a hollow needle is between about 10 micrometers and about 100 micrometers and the inner diameter of a hollow needle is between about 3 micrometers and about 80 micrometers.

Microneedles can be arranged in a variety of different patterns. In some embodiments, the microneedles are spaced apart in a uniform manner, such as in a rectangular or square grid or in concentric circles. In some embodiments, the microneedles are spaced on the periphery of the substrate, such as on the periphery of a rectangular grid. In some embodiments, the spacing depends on numerous factors, including height and width of the microneedles, the characteristics of a film to be applied to the surface of the microneedles, as well as the amount and type of a substance that is intended to be moved through the microneedles. In some embodiments, the arrangement of microneedles is a “tip-to-tip” spacing between microneedles of about 50 micrometers or more, about 100 micrometers to about 800 micrometers, or about 200 micrometers to about 600 micrometers.

### Ampoules, Vials, Pre-Filled Syringes, Etc.

In some embodiments, the compositions and/or formulations described herein may be prepared and stored in devices such as ampoules, vials, pre-filled syringes, etc., for, e.g., storage, transportation, etc., prior to uses or administration. In some embodiments, the compositions and/or formulations described herein may be dried prior to transferring into the ampoules, vials, pre-filled syringes, etc. For example, the compositions and/or formulations described herein may be lyophilized, freeze dried, spray-freeze dried, air dried, etc. to remove at least a part of water content (i.e., dehydrated). In some embodiments, the compositions and/or formulations described herein, with or without a drying process, may be vitrified to form a sugar glass structure prior to transferring into the ampoules, vials, pre-filled syringes, etc. In some embodiments, the compositions and/or formulations described herein in the ampoules, vials, pre-filled syringes, etc. are stable at a certain temperature for a certain time period. In some embodiments, the compositions and/or formulations described herein in the ampoules, vials, pre-filled syringes, etc. have increased stability at a certain temperature for a certain time period, compared to the same compositions and/or formulations without the drying step and/or the vitrification step, or not stored in the ampoules, vials, pre-filled syringes, etc. Such stability may be analyzed by measuring the remaining structure and/or function of the nucleic acid in the LNP in the compositions and/or formulations after the storage at a certain temperature for a certain time period. Examples of the temperature and the time period for storage are described in the instant disclosure. After a certain time of storage, the compositions and/or formulations described herein may be transferred out from the ampoules, vials, pre-filled syringes, etc. for further uses, such as administration to a subject, using any route described herein (e.g., through intramuscular injections).

### Pharmaceutical Compositions

In some embodiments, the microneedle devices of the disclosure include the active components (e.g., a recombinant viral replicon and/or a polypeptide) formulated as a pharmaceutical composition. In some embodiments, the pharmaceutical composition comprises a recombinant viral replicon and a pharmaceutically acceptable carrier or excipient. In some embodiments, the microneedle devices of the present disclosure include a recombinant viral replicon in water or in a buffer (e.g., a phosphate buffer, a Tris buffer, a borate buffer, a succinate buffer, a histidine buffer, or a citrate buffer), or any other pharmaceutically acceptable carrier or excipient. Any suitable pharmaceutically acceptable carriers or excipients are contemplated by the disclosure herein. In some embodiments, buffer salts, when present, are included in the 5-20 mM range. In some embodiments, pharmaceutical compositions have a pH between 5.0 and 9.5, e.g., between 6.0 and 8.0. In some embodiments, compositions include sodium salts (e.g., sodium chloride) to give tonicity. In some embodiments, a concentration of 10.+−.2 mg/ml NaCl is typical, e.g., about 9 mg/ml. In some embodiments, pharmaceutical compositions include metal ion chelators. In some embodiments, chelators prolong RNA stability by removing ions which accelerate phosphodiester hydrolysis. Examples of chelators include, but are not limited to, EDTA, EGTA, BAPTA, pentetic acid, etc., which, in some embodiments, are present at between 10-500 μM e.g., 0.1 mM. In some embodiments, a citrate salt, such as sodium citrate, act as a chelator, while advantageously also providing buffering activity. In some embodiments, pharmaceutical compositions have an osmolality of between 200 mOsm/kg and 400 mOsm/kg, e.g., between 240-360 mOsm/kg, or between 290-310 mOsm/kg. In some embodiments, pharmaceutical compositions include one or more preservatives, such as thiomersal or 2-phenoxyethanol. In some embodiments, pharmaceutical compositions are mercury-free. In some embodiments, the pharmaceutical composition is preservative-free. In some embodiments, pharmaceutical compositions are sterile or sterilized. In some embodiments, pharmaceutical compositions are non-pyrogenic, e.g., containing <1 EU (endotoxin unit, a standard measure) per dose, and in some cases <0.1 EU per dose. In some embodiments, pharmaceutical compositions contain an RNAse inhibitor. Any suitable RNAse inhibitor is contemplated for use herein, such as those sold commercially by, e.g., Life Technologies, Sigma-Aldrich, & Roche. In some embodiments, pharmaceutical compositions are prepared in unit dose form.

In some embodiments, the pharmaceutical compositions disclosed herein further comprise a small molecule immunopotentiators. In some embodiments, the pharmaceutical composition includes a TLR2 agonist (e.g., Pam3CSK4), a TLR4 agonist (e.g., an aminoalkyl glucosaminide phosphate, such as E6020), a TLR7 agonist (e.g., imiquimod), a TLR8 agonist (e.g., resiquimod) and/or a TLR9 agonist (e.g., IC31). In some embodiments, any such agonist is selected to have a molecular weight of <2000 Da.

In some embodiments, the nucleic acid compositions are stored at a temperature above freezing. In some embodiments, the compositions are stored at room temperature, such as about +20° C. to about +30° C. The compositions can be in a vitrified form (e.g., in a sugar glass state) and is stored at temperature above freezing, for example, at room temperature, such as about +20° C. to about +30° C. In some embodiments, drying and vitrification, either alone or in combination, increases stability of the nucleic acid composition at, e.g., a temperature above freezing such as at room temperature (about +20° C. to about +30° C.). Such stability increase may be, e.g., about an increase of at least 10%, 20%, 30%, 40%, 50%, 60%, 70%, 80%, 90%, 100%, 150%, 200%, 250%, 300%, 350%, 400%, 450%, 500%, 600%, 700%, 800%, 900%, 1000%, 2000%, 5000%, 10000%, or more (and all sub-values and sub-ranges there between, including endpoints), compared to the same control nucleic acid composition not vitrified. Such increase in stability may be measured by various assays, such as the translation of the mRNA in the replicon composition, the function of the translated protein/polypeptide by the mRNA, etc., after a certain time period of storage.

The vitrified compositions can be stored above freezing, for example at room temperature for extended periods of time, and still maintain greater than 15% stability for example. In some embodiments the compositions can be stored above freezing (e.g., at room temperature) for 1 week up to 1 year (or any sub value or sub range therein, inclusive of endpoints), for example, while maintaining from 15% to 100% (or any sub value or sub range therein, inclusive of endpoints) stability of the nucleic acids. In some embodiments, the vitrified nucleic acid compositions can be stored above freezing (e.g., at room temperature) for about 2 weeks to about 2 months or about 6 months (or any sub value or sub range therein, inclusive of endpoints) while maintaining greater than 50% stability, even up to 100% stability (or any sub value or sub range therein, inclusive of endpoints).

Methods of Treatment The compositions (e.g., the lipid nanoparticles (LNPs) containing RNA or other nucleic acids) provided herein, including embodiments thereof, are contemplated as providing effective treatments for diseases or disorder caused by infectious entities, such as COVID-19 and/or coronavirus-related diseases, and/or diseases or disorder caused by insufficiency of a protein, are also provided.

### A. Methods of Administration

Any of the disclosed vaccines are administered to a subject by any suitable method. Suitable methods of administration include, but are not limited to, intradermal, intramuscular, intraperitoneal, parenteral, intravenous, systemic, subcutaneous, mucosal, vaginal, rectal, intranasal, inhalation or oral. In some embodiments, parenteral administration, such as subcutaneous, intravenous or intramuscular administration, is achieved by injection. In some embodiments, injectables are prepared in conventional forms, either as liquid solutions or suspensions, solid forms suitable for solution or suspension in liquid prior to injection, or as emulsions. In some embodiments, injection solutions and suspensions are prepared from sterile powders, granules, tablets, and the like. In some embodiments, the administration is systemic. In some embodiments, the administration is local. In some embodiments, the vaccines provided herein are formulated for mucosal vaccination, such as oral, intranasal, pulmonary, rectal and vaginal. In a specific example, this is achieved by intranasal administration. In some embodiments, the administration comprises administering a vaccine as described herein comprising a sugar glass. In some embodiments, the sugar glass comprises trehalose. In some embodiments, any sugar type suitable for vitrification and sugar glass formation may be used for the compositions and formulations described herein.

In some embodiments, the administration comprises administration by a syringe. In some embodiments, the administration comprises administration by one or more needles or microneedles. In some embodiments, 100 pL-20 nL of the vaccine is administered by each microneedle. In some embodiments, the administration comprises intranasal, intradermal, intramuscular, skin patch, topical, oral, subcutaneous, intraperitoneal, intravenous, systemic, or intrathecal administration.

In some embodiments, the administration comprises rubbing or wiping a subject's skin with a wipe at a site of administration prior to injecting the vaccine with a needle or microneedle. In some embodiments, the wipe is a cleaning wipe. In some embodiments, the wipe is an imiquimod wipe. In some embodiments, the imiquimod wipe is rubbed into a subject's skin at the subject's site of administration such that the imiquimod is rubbed into the skin at the site be vaccinated prior to injecting the vaccine into the site of administration with a microneedle device.

Some embodiments include microneedle administration. Some embodiments include skin patch administration. Some embodiments include microneedle skin patch administration. In some embodiments, microneedles are placed on cleaned skin of the subject and pressed into the skin. In some embodiments, the microneedle skin patch comprises a dose of vaccine loaded on or in the microneedles in a liquid dispensing step. In some embodiments, microfluidic dispensing of 10-20 nL per microneedle is used.

In some embodiments, the vaccines are dried in a well inside each microneedle. In some embodiments, this keeps the microneedles sharp enough for a light force of under 10 Newtons to be successful in delivery. In some embodiments, the vaccines are dried outside each microneedle. In some embodiments, a microneedle array is used for administration.

In some embodiments, vaccines are packaged onto microneedles. In some embodiments, vaccines are packaged or embedded into microneedles. In some embodiments, the vaccine is dehydrated after packaging into or onto the microneedle. In some embodiments, the microneedle is packaged individually at a unit dose of vaccine. In some embodiments, the unit dose is effective in inducing an immune response in a subject to the antigen. In some embodiments, the unit dose is effective in inducing an immune response in a subject to the antigen after storage for at least about one week [e.g., about or more than about 1, 2, 3, 4, 6, 8, 12, or more (and all sub-values and sub-ranges there between, including endpoints) weeks] at room temperature. In some embodiments, the unit dose is effective in inducing an immune response in a subject to the antigen after storage for at least about one month [e.g., about or more than about 1, 2, 3, 4, 5, 6, 8, 10, 12, or more (and all sub-values and sub-ranges there between, including endpoints) months] at room temperature. In some embodiments, the vaccine is present in an amount effective to induce an immune response in the subject to the antigen. In some embodiments, the microneedle administration is painless.

In some embodiments, the vaccine antigen is expressed in terms of an amount of antigen per dose. In some embodiments, a dose has 100 mg antigen or total protein (e.g., from 1-100 mg, such as about 1 mg, 5 mg, 10 mg, 25 mg, 50 mg, 75 mg or 100 mg). In some embodiments, expression is seen at much lower levels (e.g., 1 mg/dose, 100 ng/dose, ng/dose, or 1 ng/dose).

### B. Timing of Administration

In some embodiments, the method comprises multiple administrations or doses 10 of a vaccine as described herein. In some embodiments, a disclosed vaccine is administered as a single or as multiple doses (e.g., boosters). In some embodiments, the first administration is followed by a second administration. In some embodiments, the second administration is with the same, or with a different vaccine than the vaccine administered. In some embodiments, the second administration is with the same vaccine as the first vaccine administered. In some embodiments, the second administration is with a vaccine comprising a different vaccine than the first vaccine administered. In some embodiments, if the first vaccine includes a first HA subtype and a second HA subtype, the second vaccine comprises a third HA subtype and a fourth HA subtype, wherein all four subtypes are different (such as four of H1, H2, H3, H5, H7, and H9).

In some embodiments, the vaccines containing two or more viral replicons are administered as multiple doses, such as 2, 3, 4, 5, 6, 7, 8, 9, or 10 doses (such as 2-3 doses). In some embodiments, the timing between the doses is at least 1 week, at least 2 weeks, at least 3 weeks, at least 4 weeks, at least 6 weeks, at least 8 weeks, at least 12 weeks, at least 2 months, at least 3 months, at least 4 months, at least 5 months, at least 6 months, at least 1 year, at least 2 years, or at least 5 years, such as 1˜4 weeks, 2-3 weeks, 1-6 months, 2-4 months, 1 week, 2 weeks, 3 weeks, 4 weeks, 5 weeks, 6 weeks, 7 weeks, 8 weeks, 9 weeks, 10 weeks, 12 weeks, 1 month, 2 months, 3, months, 4, months, 5 months, 6 months, 1 year, 2 years, 5 years, or 10 years, or combinations thereof (such as where there are at least three administrations, wherein the timing between the first and second, and second and third doses, are in some embodiments the same or different).

### C. Dosages

In some embodiments, the method comprises administering a dose of 1 pg, 10 pg, 25 pg, 100 pg, 250 pg, 500 pg, 750 pg, 1 ng, 5 ng, 10 ng, 15 ng, 20 ng, 25 ng, 50 ng, 100 ng, 250 ng, 500 ng, 1 mg, 10 mg, 50 mg, 100 mg, 500 mg, 1 mg, 5 mg, 10 mg, 50 mg, 100 mg, 500 mg, or 1 g of the vaccine (e.g. samRNA-containing LNPs), or a range of doses defined by any two of the aforementioned doses.

In some embodiments, the subject is administered (e.g., intravenous or systemic) about 1 to about 100 mg of a vaccine or each of vaccines, such as about 1 mg to about 50 mg, 1 mg to about 25 mg, 1 mg to about 5 mg, about 5 mg to about 20 mg, or about 10 mg to about 15 mg of a vaccine or each of vaccines. In some embodiments, the subject is administered about 15 mg of a vaccine or each of vaccines. In some embodiments, the subject is administered about 10 mg of a vaccine or each of vaccines. In some embodiments, the subject is administered about 20 mg of a vaccine or each of vaccines. In some embodiments, the subject is administered about 1 mg or 2 mg of a vaccine or each of vaccines.

In some embodiments, the dose administered to a subject is sufficient to induce a beneficial therapeutic response in the subject over time, or to inhibit or prevent an infection. In some embodiments, the dose varies from subject to subject, or is administered depending on the species, age, weight and general condition of the subject, the severity of an infection being treated, and/or the particular vaccine being used and its mode of administration.

The dosages described herein may be determined according to the administration routes or methods. For example, microneedles and arrays described herein, such as Vaxipatch, provide a way to administer to a subject compositions and formulations described herein in a relative lower dosage. In some embodiments, the dosage for the compositions and formulations administered by these routes may be about 1 pg, 10 pg, 25 pg, 100 pg, 250 pg, 500 pg, 750 pg, 1 ng, 5 ng, 10 ng, 15 ng, 20 ng, 25 ng, 50 ng, 100 ng, 250 ng, 500 ng, 1 mg, 10 mg, 50 mg, 100 mg, 500 mg, 1 mg, or more (and all sub-values and sub-ranges there between, including endpoints) of the compositions and formulations (e.g. vaccines comprising nucleic acids, such as RNAs), or a range of doses defined by any two of the aforementioned doses. In contrast, traditional administration routs, such as IM injections, may require a relatively higher dosage. In some embodiments, the dosage for the compositions and formulations administered by these routes may be about 1 ng, 5 ng, 10 ng, 15 ng, 20 ng, 25 ng, 50 ng, 100 ng, 250 ng, 500 ng, 1 mg, 10 mg, 50 mg, 100 mg, 500 mg, 1 mg, 5 mg, 10 mg, 50 mg, 100 mg, 500 mg, 1 g, or more (and all sub-values and sub-ranges there between, including endpoints), or a range of doses defined by any two of the aforementioned doses.

### Definitions

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

The abbreviations used herein have their conventional meaning within the chemical and biological arts. The chemical structures and formulae set forth herein are constructed according to the standard rules of chemical valency known in the chemical arts.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, NY 1994); Sambrook et al., MOLECULAR CLONING, A LABORATORY MANUAL, Cold Springs Harbor Press (Cold Springs Harbor, NY 1989). Any methods, devices and materials similar or equivalent to those described herein can be used in the practice of this invention. The following definitions are provided to facilitate understanding of certain terms used frequently herein and are not meant to limit the scope of the present disclosure.

The terms “polynucleotide,” “nucleotide,” “nucleotide sequence,” “nucleic acid,” and “oligonucleotide” are used interchangeably. They refer to a polymeric form of nucleotides of any length, either deoxyribonucleotides or ribonucleotides, or analogs thereof. The following are non-limiting examples of polynucleotides: coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. In some embodiments, a polynucleotide comprises one or more modified nucleotides, such as methylated nucleotides and nucleotide analogs. In some embodiments, modifications to the nucleotide structure are imparted before or after assembly of the polymer. In some embodiments, the sequence of nucleotides is interrupted by non-nucleotide components. In some embodiments, the polynucleotide is further modified after polymerization, such as by conjugation with a labeling component.

The terms “polypeptide,” “peptide,” and “protein” are used interchangeably herein to refer to polymers of amino acids of any length. In some embodiments, a polypeptide is any protein, peptide, protein fragment, or component thereof. In some embodiments, a polypeptide is a protein naturally occurring in nature or a protein that is ordinarily not found in nature. In some embodiments, a polypeptide consists largely of the standard twenty protein-building amino acids or it is modified to incorporate non-standard amino acids. In some embodiments, a polypeptide is modified, typically by the host cell, for example, by adding any number of biochemical functional groups, including phosphorylation, acetylation, acylation, formylation, alkylation, methylation, lipid addition (e.g., palmitoylation, myristoylation, prenylation, etc.) and carbohydrate addition (e.g., N-linked and O-linked glycosylation, etc.). In some embodiments, polypeptides undergo structural changes in the host cell such as the formation of disulfide bridges or proteolytic cleavage.

In general, “sequence identity” refers to an exact nucleotide-to-nucleotide or amino acid-to-amino acid correspondence of two polynucleotides or polypeptide sequences, respectively. Typically, techniques for determining sequence identity include determining the nucleotide sequence of a polynucleotide and/or determining the amino acid sequence encoded thereby, and comparing these sequences to a second nucleotide or amino acid sequence. Any suitable technique is contemplated by the disclosure herein. In some embodiments, two or more sequences (polynucleotide or amino acid) are compared by determining their “percent identity.” In some embodiments, ranges of desired degrees of sequence identity are approximately 80% to 100% and integer values there between. In some embodiments, the percent identities between a disclosed sequence and a claimed sequence are at least 80%, at least 85%, at least 90%, at least 95%, or at least 98%.

The terms “subject,” “individual,” and “patient” are used interchangeably herein to refer to a vertebrate, preferably a mammal, more preferably a human. In some embodiments, mammals include, but are not limited to: murines, simians, humans, farm animals, sport animals, and pets. In some embodiments, tissues, cells, and their progeny of a biological entity obtained in vivo or cultured in vitro are encompassed. None of these terms, as used herein, entail supervision of a medical professional.

As used herein, the term “about” is used to indicate that a value includes the standard deviation of error for the device or method being employed to determine the value.

Unless defined otherwise, all technical and scientific terms used herein have the same meaning as is commonly understood by one of skill in the art to which the claimed subject matter belongs. It is to be understood that the foregoing general description and the following detailed description are examples and for the explanatory purpose only and are not restrictive of any subject matter claimed. In this application, the use of the singular includes the plural unless specifically stated otherwise. It must be noted that, as used in the specification and the appended claims, the singular forms “a,” “an,” and “the” include plural referents unless the context clearly dictates otherwise. In this application, the use of “or” means “and/or” unless stated otherwise. Furthermore, use of the term “including” as well as other forms, such as “include,” “includes,” and “included,” is not intended to be limited solely to the recited items. The section headings used herein are for organizational purposes only and are not to be construed as limiting the subject matter described.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The terms “SARS-COV-2 spike protein” and “SARS-COV-2 protein” as used herein include any of the recombinant or naturally-occurring forms of SARS-COV-2 Spike protein, or variants or homologs thereof that maintain SARS-COV-2 spike protein's activity (e.g. within at least 50%, 80%, 90%, 95%, 96%, 97%, 98%, 99% or 100% activity compared to SARS-COV-2 spike protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring SARS-COV-2 spike protein.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The terms “plasmid”, “vector” or “expression vector” refer to a nucleic acid molecule that encodes for genes and/or regulatory elements necessary for the expression of genes. Expression of a gene from a plasmid can occur in cis or in trans. If a gene is expressed in cis, the gene and the regulatory elements are encoded by the same plasmid. Expression in trans refers to the instance where the gene and the regulatory elements are encoded by separate plasmids.

The terms “transfection”, “transduction”, “transfecting” or “transducing” can be used interchangeably and are defined as a process of introducing a nucleic acid molecule or a protein to a cell. Nucleic acids are introduced to a cell using non-viral or viral-based methods. The nucleic acid molecules may be gene sequences encoding complete proteins or functional portions thereof. Non-viral methods of transfection include any appropriate transfection method that does not use viral DNA or viral particles as a delivery system to introduce the nucleic acid molecule into the cell. Examples of non-viral transfection methods include calcium phosphate transfection, liposomal transfection, nucleofection, sonoporation, transfection through heat shock, magnetifection and electroporation. In some embodiments, the nucleic acid molecules are introduced into a cell using electroporation following standard procedures well known in the art. For viral-based methods of transfection any useful viral vector may be used in the methods described herein. Examples for viral vectors include, but are not limited to retroviral, adenoviral, lentiviral and adeno-associated viral vectors. In some embodiments, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include, but are not limited to, yeast cells and cells derived from plants and animals, for example mammalian, insect (e.g., spodoptera) and human cells.

The term “recombinant” when used with reference, e.g., to a cell, nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all. Transgenic cells and plants are those that express a heterologous gene or coding sequence, typically as a result of recombinant methods.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source.

The term “exogenous” refers to a molecule or substance (e.g., a compound, nucleic acid or protein) that originates from outside a given cell or organism. For example, an “exogenous promoter” as referred to herein is a promoter that does not originate from the cell or organism it is expressed by. Conversely, the term “endogenous” or “endogenous promoter” refers to a molecule or substance that is native to, or originates within, a given cell or organism.

One of skill in the art will understand which standard controls are most appropriate in a given situation and be able to analyze data based on comparisons to standard control values. Standard controls are also valuable for determining the significance (e.g. statistical significance) of data. For example, if values for a given parameter are widely variant in standard controls, variation in test samples will not be considered as significant.

“Patient” or “subject in need thereof” refers to a living organism suffering from or prone to a disease or condition that can be treated by administration of a composition or pharmaceutical composition as provided herein. Non-limiting examples include humans, other mammals, bovines, rats, mice, dogs, monkeys, goat, sheep, cows, deer, and other non-mammalian animals. In some embodiments, a patient is human.

As used herein, “treating” or “treatment of” a condition, disease or disorder or symptoms associated with a condition, disease or disorder refers to an approach for obtaining beneficial or desired results, including clinical results. Beneficial or desired clinical results can include, but are not limited to, alleviation or amelioration of one or more symptoms or conditions, diminishment of extent of condition, disorder or disease, stabilization of the state of condition, disorder or disease, prevention of development of condition, disorder or disease, prevention of spread of condition, disorder or disease, delay or slowing of condition, disorder or disease progression, delay or slowing of condition, disorder or disease onset, amelioration or palliation of the condition, disorder or disease state, and remission, whether partial or total. “Treating” can also mean prolonging survival of a subject beyond that expected in the absence of treatment. “Treating” can also mean inhibiting the progression of the condition, disorder or disease, slowing the progression of the condition, disorder or disease temporarily, although in some instances, it involves halting the progression of the condition, disorder or disease permanently. As used herein the terms treatment, treat, or treating refers to a method of reducing the effects of one or more symptoms of a disease or condition characterized by expression of the protease or symptom of the disease or condition characterized by expression of the protease. Thus in the disclosed method, treatment can refer to a 10%, 20%, 30%, 40%, 50%, 60%, 70%, 80%, 90%, or 100% reduction in the severity of an established disease, condition, or symptom of the disease or condition. For example, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. Further, as used herein, references to decreasing, reducing, or inhibiting include a change of 10%, 20%, 30%, 40%, 50%, 60%, 70%, 80%, 90% or greater as compared to a control level and such terms can include but do not necessarily include complete elimination.

The terms “dose” and “dosage” are used interchangeably herein. A dose refers to the amount of active ingredient given to an individual at each administration. The dose will vary depending on a number of factors, including the range of normal doses for a given therapy, frequency of administration; size and tolerance of the individual; severity of the condition; risk of side effects; and the route of administration. One of skill will recognize that the dose can be modified depending on the above factors or based on therapeutic progress. The term “dosage form” refers to the particular format of the pharmaceutical or pharmaceutical composition, and depends on the route of administration. For example, a dosage form can be in a liquid form for nebulization, e.g., for inhalants, in a tablet or liquid, e.g., for oral delivery, or a saline solution, e.g., for injection.

By “therapeutically effective dose or amount” as used herein is meant a dose that produces effects for which it is administered (e.g. treating or preventing a disease). The exact dose and formulation will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Remington: The Science and Practice of Pharmacy, 20th Edition, Gennaro, Editor (2003), and Pickar, Dosage Calculations (1999)). For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more (and all sub-values and sub-ranges there between, including endpoints) effect over a standard control. A therapeutically effective dose or amount may ameliorate one or more symptoms of a disease. A therapeutically effective dose or amount may prevent or delay the onset of a disease or one or more symptoms of a disease when the effect for which it is being administered is to treat a person who is at risk of developing the disease.

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example COVID-19 or other coronavirus-related therapies. The compounds of the invention can be administered alone or can be co-administered to the patient. Co-administration is meant to include simultaneous or sequential administration of the compounds individually or in combination (more than one compound). Thus, the preparations can also be combined, when desired, with other active substances (e.g. to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

As used herein, the term “pharmaceutically acceptable” is used synonymously with “physiologically acceptable” and “pharmacologically acceptable”. A pharmaceutical composition will generally comprise agents for buffering and preservation in storage, and can include buffers and carriers for appropriate delivery, depending on the route of administration.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “pharmaceutically acceptable salt” refers to salts derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

The pharmaceutical preparation is optionally in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The unit dosage form can be of a frozen dispersion.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

### Example 1: Preparation of samRNA-Containing LNPs

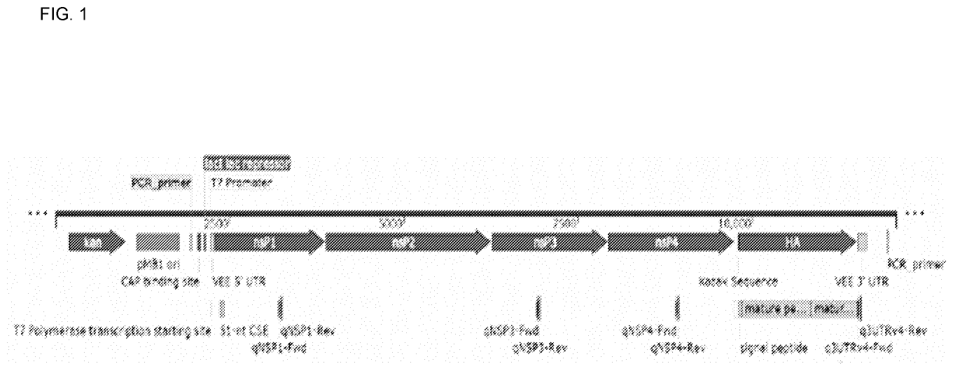

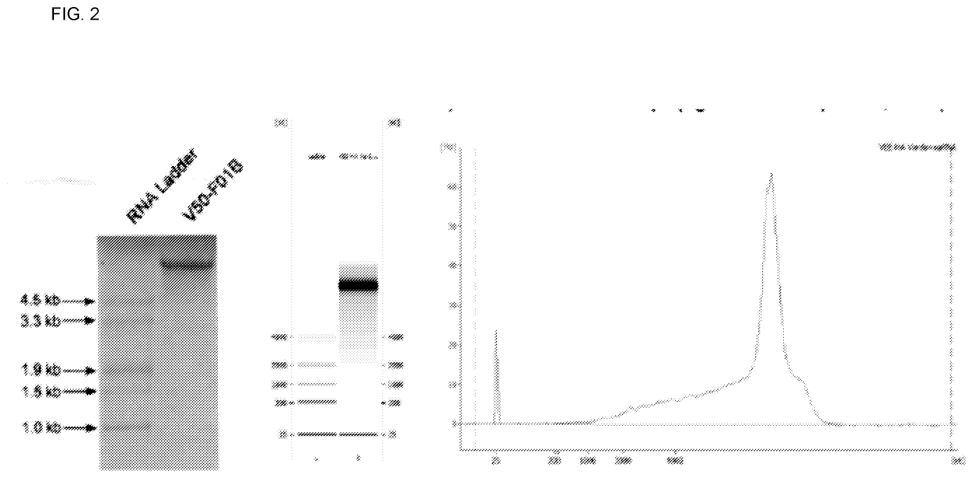

Venezuelan equine encephalitis virus (VEEV)-derived self-amplifying RNA (samRNA) replicons were produced at TriLink BioTechnologies (San Diego, CA) by in vitro transcription of mRNA (IVTmRNA). DNA of the gene of interest (GOI) was inserted into a DNA plasmid so as to produce an mRNA with the T7 RNA polymerase. The reaction contains linearized DNA template, T7 polymerase, four RNA nucleoside triphosphates (with or without modifications such as pseudouridine) and salts. The general structure of the template DNA plasmid constructs is shown in FIG. 1. DNA plasmid constructs encoding the GOIs, EGFP, luciferase, influenza hemagglutinin (HA) of the influenza H1N1/CA09 strain and HBSAg were produced. Large mRNAs were in vitro synthesized by TriLink for all of these constructs. The production of VEEV-HA CA/09 construct was confirmed, as in FIG. 2, in which the left panel shows an agarose gel image illustrating the electrophoresis result of the produced VEEV-HA CA/09 construct (the dark band in the right lane in the image), the middle panel shows an image from an Agilent BioAnalyzer for the construct, and the right panel shows a scan result by the Agilent BioAnalyzer.

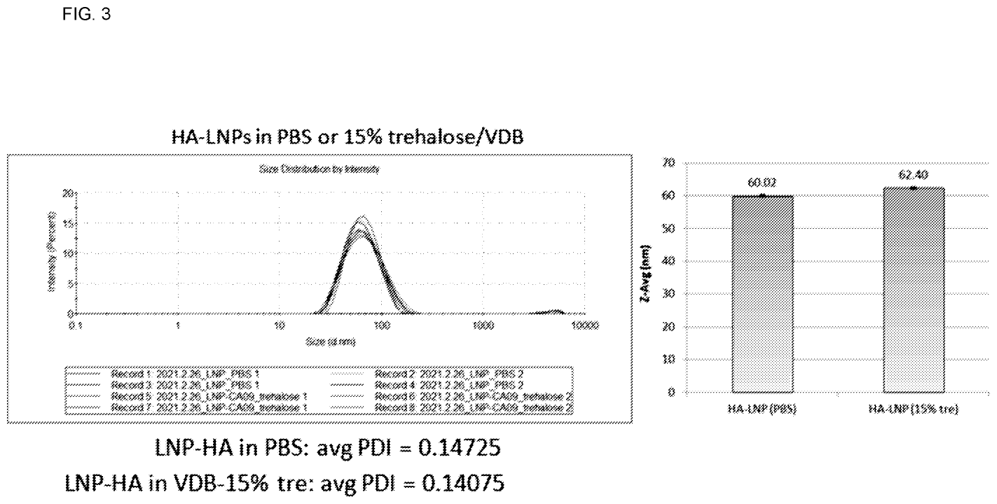

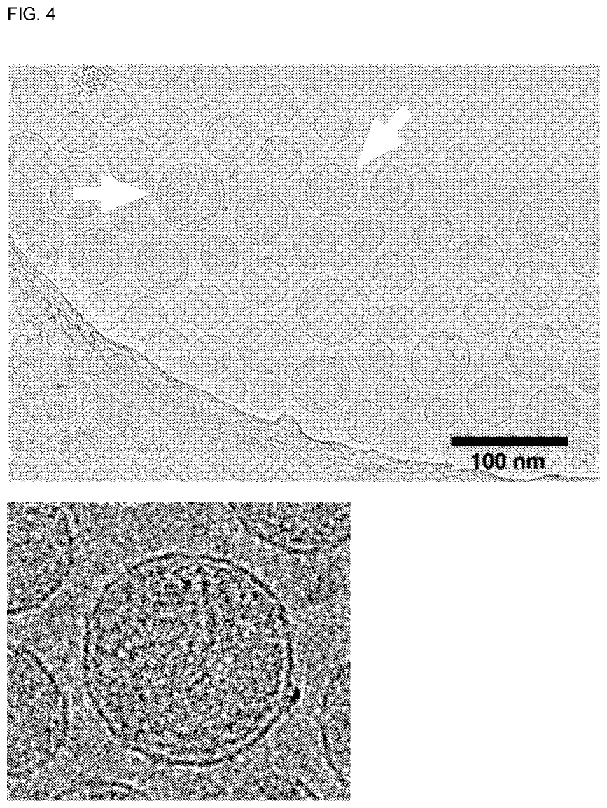

samRNA LNPs were prepared by NanoAssemblr, mixing the dried lipids (cholesterol, DOPC, DMG-PEG-2000 and C12-200) in ethanol at 10 mg/mL (0.25 mL) with 0.75 mL of sodium acetate buffer (pH 4) at a flow rate of 8 mL/min. Ethanol was removed, buffer was changed to PBS (pH 7.4), and LNPs were concentrated to a final 125 μL using the ultrafiltration Amicon tube (MWCO: 100 kDa) in the presence of 15% trehalose in PBS. These samRNA LNPs were imaged by DLS (Dynamic Light Scattering, Malvern Zetasizer Nano ZS; FIG. 3) and cryoelecton microscopy (UC Davis; FIG. 4). As a result, the measured dynamic light scattering (DLS) from the Malvern Zetasizer of LNPs increased from 60 nm to 62 nm, upon formulation with 15% trehalose. Thus, these LNPs are compatible with trehalose in formulations. Generally, LNPs having a reading over 200 may be inactive.

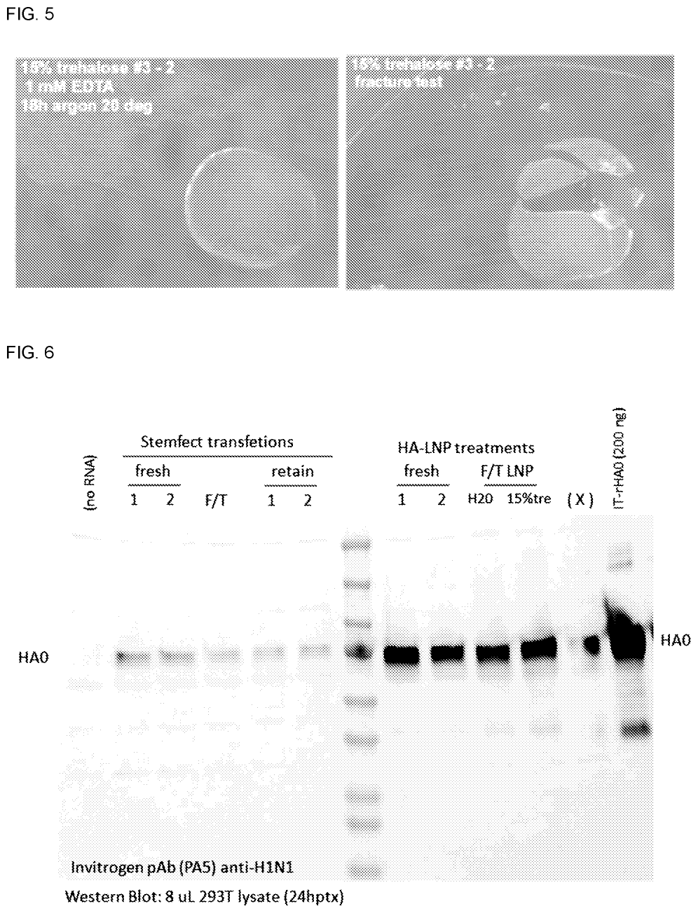

These samRNA LNPs dry into a stable sugar glass as shown by the fracture test (FIG. 5).

Fully functional samRNA LNPs were produced, measured in vitro on 293T cells. FIG. 6 contains a PAGE image showing HA production in rats following IM injection of samRNA LNPs.

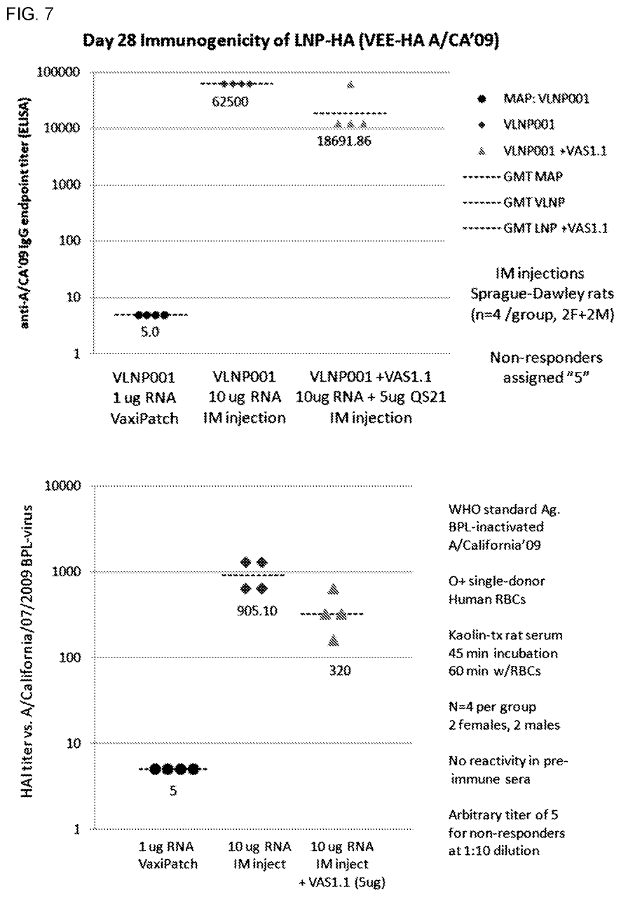

samRNA LNPs were also shown to be highly active following intramuscular (IM) injection. FIG. 7 shows ELISA titers (the top panel) and the HAI titers (the bottom panel) of 28-day bleeds following the IM injection of LNPs containing 10 μg of samRNA. Note the exceedingly high titers of HAI antibodies (the bottom panel). Also note the adjuvant QS-21 was of no help and actually decreased titers (the top panel).

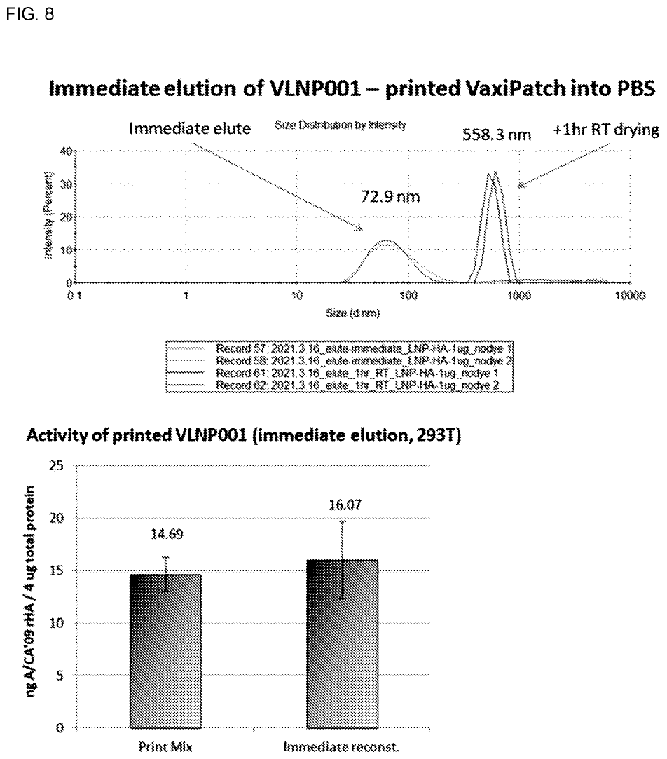

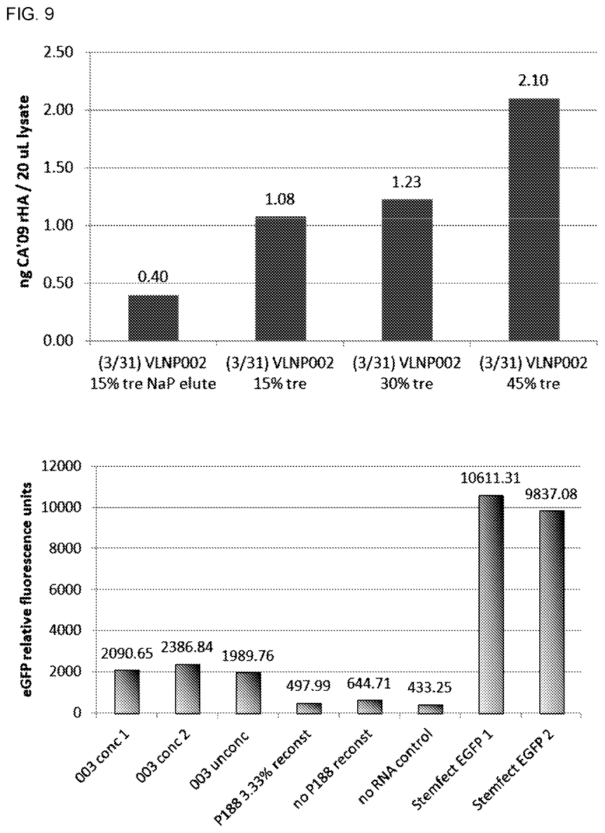

FIGS. 8 and 9 show that tacky dry freshly printed samRNAs are fully active. However, after continued drying for 1 hour into a sugar glass, over 90% of the activity was lost. Importantly, a residual single-digit percentage of samRNA LNP activity was retained upon full drying at room temperature. LNP fusion and lipid component rearrangement appeared to be the problem.

Formulations have been prepared with the goal of increasing the retained activity. One goal is to deliver mRNA vaccines on VaxiPatch in a room temperature stable format. RNA vaccines formulations as described herein incorporated with VaxiPatch have been shown to be room temperature stable for 28 days or more, retaining substantial activity, for example, in terms of ability to be rehydrated and to be functional RNA. For mRNA-based vaccines, real-time as well as multiple temperature Arrhenius plot accelerated shelf-life studies are used.

### Example 2: Discovering Excipients for Formulating LNPs

LNPs were created from the ionizable lipid C12-200, resulting in a slightly larger size with an average surface charge of neutral toward positive values, compared to those prepared from DLin-MC3-DMA that exhibit negative zeta potential values. P188 and HES were used to formulate LNPs for vitrification, sugar glass formation and activity of mRNA LNPs.

Mixtures of excipients added to mRNA LNPs may help keep the LNPs from fusing into large inactive particles. Poloxomer 188 (P188, Pluronic F68, BASF Corporation) was found to be one of such excipients. P188 is nonionic linear copolymer having an average molecular weight of 8400 Daltons and is also referred to as PLURONIC F68, FLOCOR and RheothRx. The copolymer was approved by the FDA nearly 50 years ago as a therapeutic reagent to reduce viscosity in the blood before transfusions. The most well documented characteristic of P188 is its ability to repair damaged cell membranes by mechanisms that are not entirely clear. However, P188 may act by increasing the lipid packing density. It is believed that P188 functions by way of direct incorporation into the phospholipid bilayer, and the process is modulated by the lipid membrane's surface tension as demonstrated by in vitro lipid monolayer experiments. In current experiments, P188 may render the LNP formulation viscous, when concentrated for, e.g., microneedle arrays. However, a LNP formulation comprising P188 in a vial, without a need to concentrate, was not viscous.

Hydroxyethyl cellulose (HEC) is also FDA approved for hypovolemia. HES130 (Volulyte®) and HES200 (Pentaspan®) were tested in experimentation described in this Example. Hypovolemia often occurs in patients presenting with severe trauma or postoperatively after major surgery. Effective management of blood volume replacement is thus paramount to ensure adequate peripheral tissue perfusion and oxygenation. HES is also used in acute renal failure is a frequent complication of sepsis.

In this Example, two types of LNPs containing 2.5 mg lipids and 85.6 μg of either HA-samRNA or EGFP-samRNA and vacant liposomes with the lipid formulation similar to LNPs were prepared from C12-200 with the lipid composition and concentration reported previously. For LNPs, dried lipids were dissolved in ethanol at 10 mg/mL (0.25 mL) and combined with 0.75 mL of either VEE-HA-samRNA (9412 bp, 86 μg) or VEE-EGFP-samRNA (8435 bp, 86 μg) in 25 mM sodium acetate buffer (pH 4) using the Benchtop NanoAssemblr to create VEE-HA-samRNA-LNP and VEE-EGFP-samRNA-LNPs, each at a final volume of 700 μL. Liposomes were prepared by NanoAssemblr, mixing the dried lipids in ethanol at 10 mg/mL (0.25 mL) with 0.75 mL of PBS (pH 7.4) at a flow rate of 8 mL/min. Ethanol was removed, buffer was changed to PBS (pH 7.4), and LNPs were concentrated to a final 125 μL using the ultrafiltration Amicon tube (MWCO: 100 kDa) in the presence of 15% trehalose in PBS.

The concentrations of HA-samRNA or EGFP-samRNA in C12-200-LNPs and HAsamRNA were quantified by the Qubit® RNA BR Assay Kits prior to drying and addition to the cells. A fraction of LNPs was then dried in the drying buffers described as follows:
DB1: 15 μL LNPs (containing 15% trehalose)+7.5 μL of 15% trehalose+7.5 μL of 20% P188 (11.25% trehalose and 5% P188);
DB2: 15 μL LNPs (containing 15% trehalose)+15 μL of 20% P188 (7.5% trehalose and 10% P188);
DB3: 15 μL LNPs (containing 15% trehalose)+15 μL 10% HES (130) (7.5% trehalose and 5% HES130); and
DB4: 15 μL LNPs (containing 15% trehalose)+15 μL 10% HES (200) (7.5% trehalose and 5% HES200).

The mixtures were dried in a total volume of 30 μL in the cap of clean 1.5-mL Eppendorf tubes for 24 h. The dried LNPs, using different DBs, were reconstituted in 30 μL of the corresponding DB in the inverted Eppendorf tubes at 37° C. for 10 min. The reconstituted LNPs were recovered at the bottom of the tube following a gentle spin.

### Characterization of LNPs Before and After Reconstitution

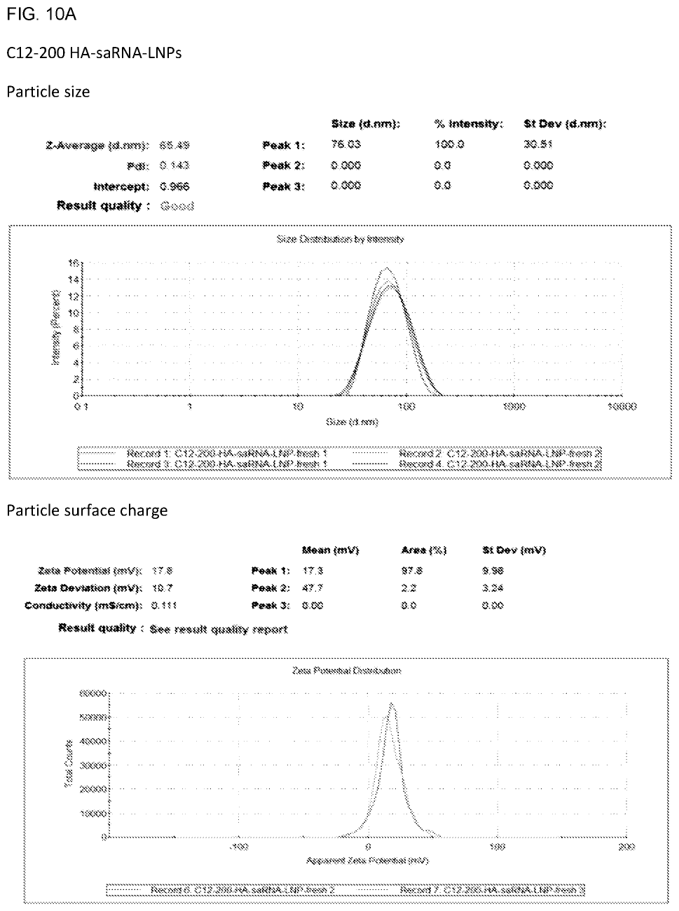

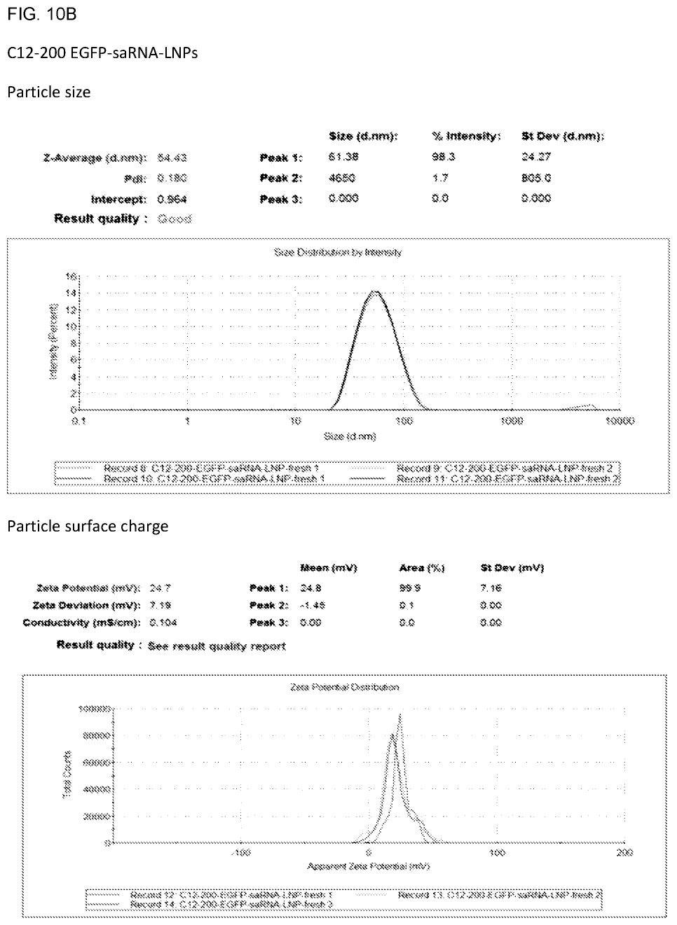

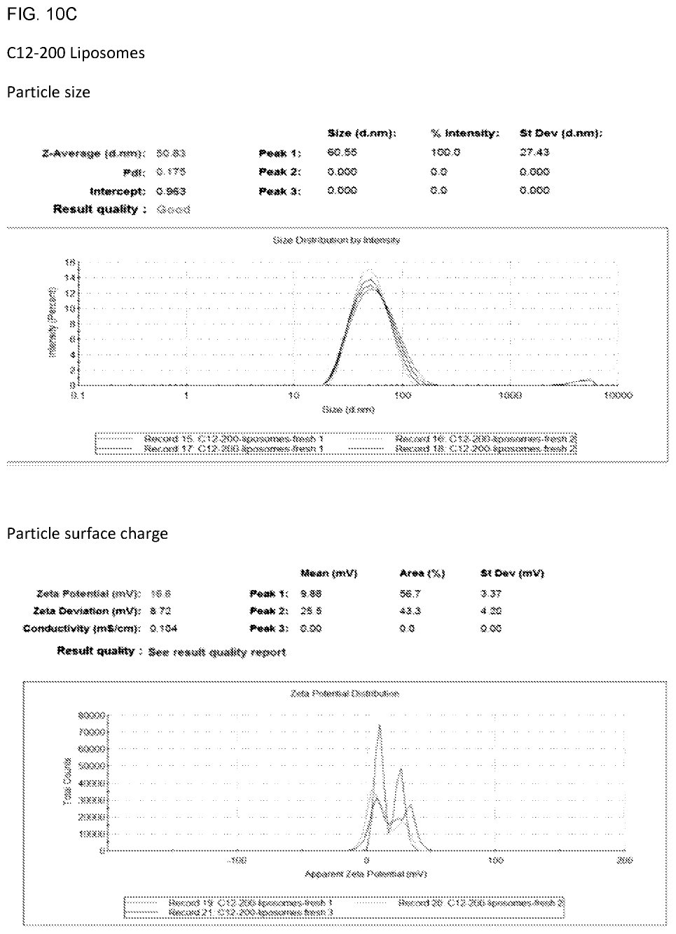

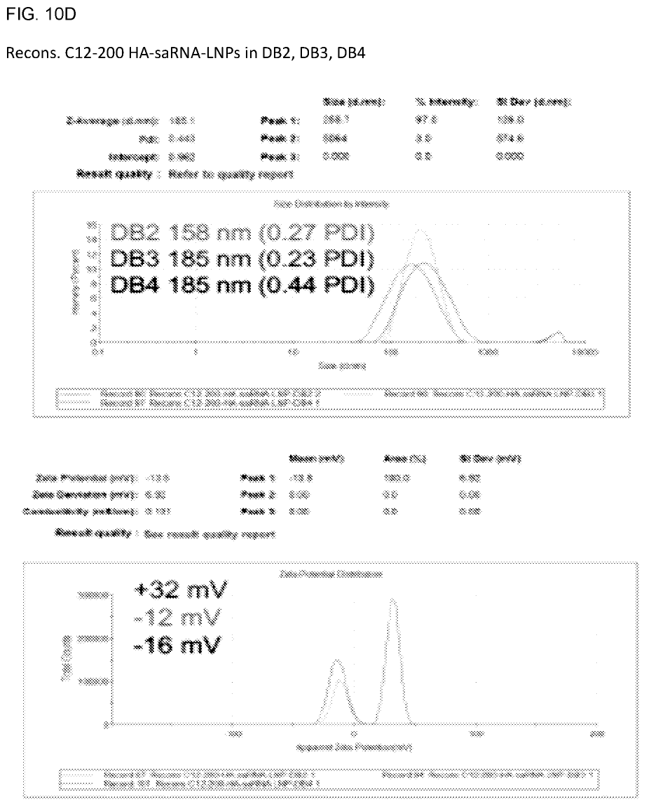

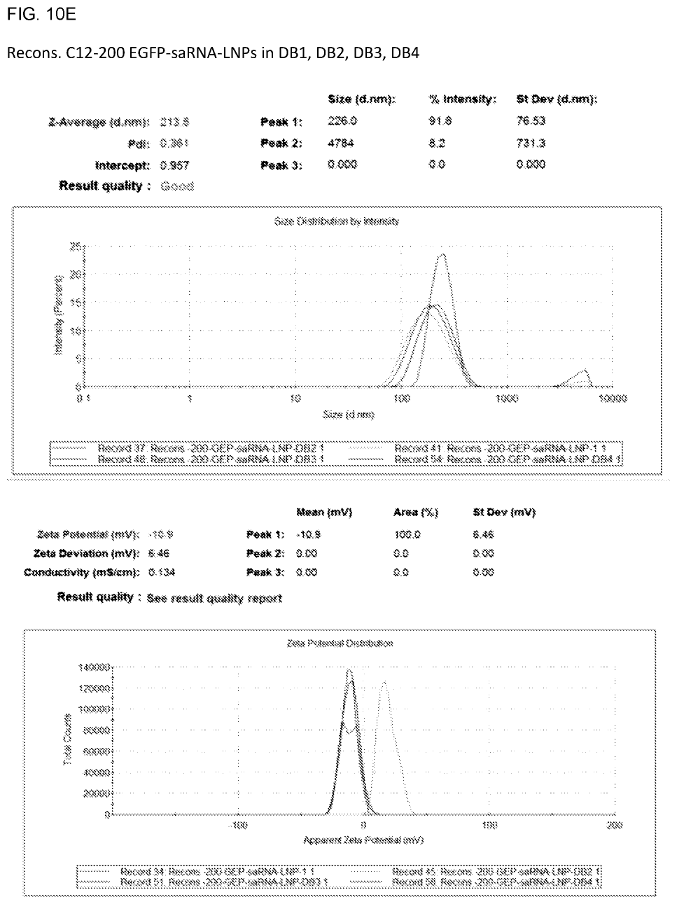

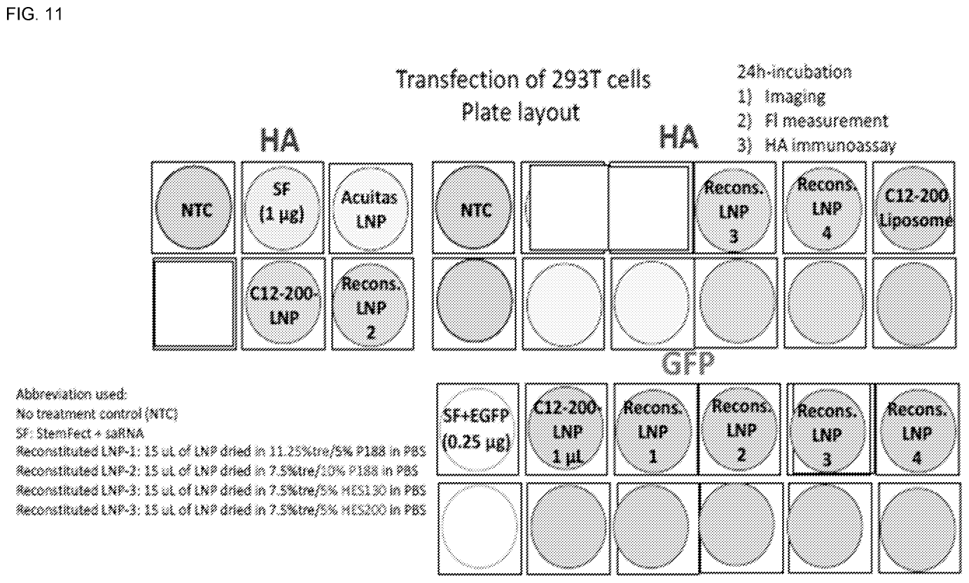

FIGS. 10A-10E compare the size and zeta potential values of the LNPs, with or without reconstitution in different drying buffers (DBs). All LNPs displayed a unimodal size distribution with the average size in the range of 52-72 nm and positive particle charges of +8-22 mV. Vacant liposomes had an average size of 50 nm with +16 mV zeta potential. LNPs reconstituted at various conditions appeared as a single peak on both size and zeta potential distribution.

Interestingly, LNPs dried in drying buffers containing P188 (e.g., DB1 and DB2) or HES (e.g., DB3 and DB4) after 24 h in the vacuum chamber at ambient temperature produced different texture sugar glasses. LNPs, when dried in the presence of HES, produced more uniformly distributed and transparent textures in the formed sugar glass than those dried in the presence of P188.

Quantification of mRNA Loading in LNPs and the Encapsulation Efficiency

RNA loading was then determined for the LNPs listed in Table 1.

TABLE 1

| | | Free/membrane- | | | | Total RNA (× | | |
|---|---|---|---|---|---|---|---|---|
| | | associated RNA | | Total RNA | | dilution factor) | | Encapsulation |
| | | ng/μL | | ng/μL | | μg/μL | | Efficiency |
| LNP Simulation | Date | Avg | Std | Avg | Std | Avg | Std | % |
| C12-200 MA-ssRNA LNP | Jul. 28, 2021 | 15. [symbol] | 0.89 | 75.41 | 0. [symbol] | 0. [symbol] | 0.01 | 79 |
| C12-200-EGFP-ssRNA LNP | Jul. 28, 2021 | 8.04 | 1.30 | [symbol] 8.08 | 0.81 | 0.54 | 0.00 | 88 |
| C12-200-Liposomes | Jul. 28, 2021 | 0.00 | 0.00 | 0.00 | 0.10 | 0.00 | 0.00 | 0 |
| C12-200-EGFP-ssRNA LNP | Jul. 14, 2021 | 7.69 | 1.21 | 57.95 | 2. [symbol] | 0.45 | 0.02 | 87 |
| C12-200-MA-ssRNA LNP | Jul. 20, 2021 | 17.20 | 0.72 | 76.48 | 0.61 | 0.61 | 0.00 | 78 |

[symbol]

indicates data missing or illegible when filed

For C12-200-LNPs the encapsulation efficiency was relatively low (78-79%) for HA-samRNA (78-79%) but much higher for EGFP-samRNA (87-88%). C12-200-VEE-HA-samRNA-LNPs and C12-200-VEE-EGFP samRNA-LNPs had RNA concentrations of 0.6 and 0.54 μg/μL, respectively, which were close to the estimated value of 0.7 μg/μL. A fraction of RNA that was not properly packaged within LNPs was higher for HA-samRNA-LNPs than EGFP-samRNA-LNPs. This RNA fraction could be either free RNA or membrane-associated RNA.

### Transfection in 293T Cell Culture

The expression level of HA was examined in a 6-well plate and in 8 wells of a 24-well plate and that of EGFP in 16 wells of a 24-well plate against StemFect (SF) transfection reagent and the cells with no treatment (NTC). Freshly thawed 293T cells were cultured in one 6-well and one 24-well plates at 7.5×10⁵ and 1.25×10⁵ cells/well, respectively. LNPs dried in various buffers were then reconstituted in the corresponding buffers prior to addition to the cells. The complete media was replaced with reduced serum media (OptiMEM) 15 min prior to the treatments. Since the dried LNPs are reconstituted in a two-fold higher volume with some loss, three times higher RNA concentrations were used for all the reconstituted LNPs compared to non-reconstituted LNPs. In addition, for consistency among various treatments, all samples were added at 100 μL/well to cells in the 6-well plate and at 10 μL/well in the 24-well plate. Following 2 h incubation of cells with various treatments in OptiMEM, an equal volume of the complete media was added per well and the cells were incubated for another 22 h.

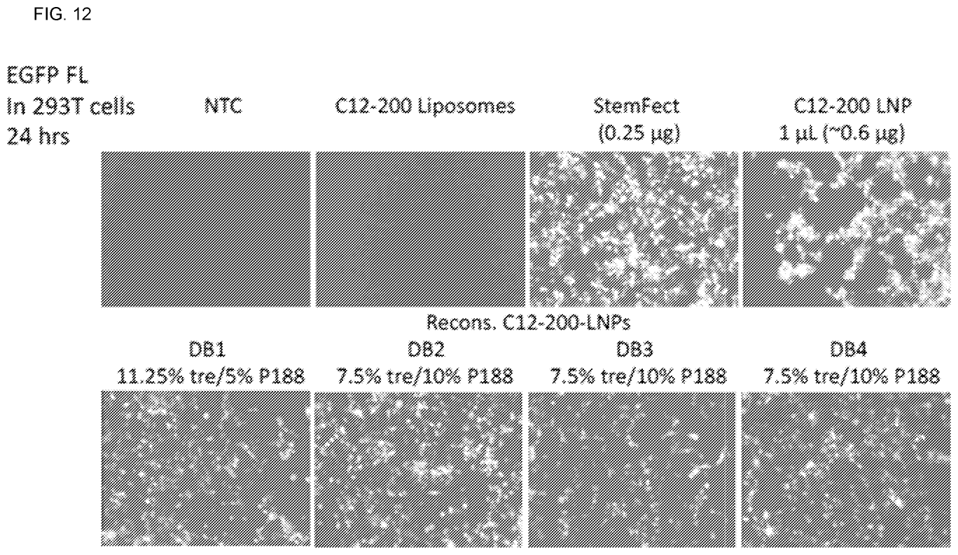

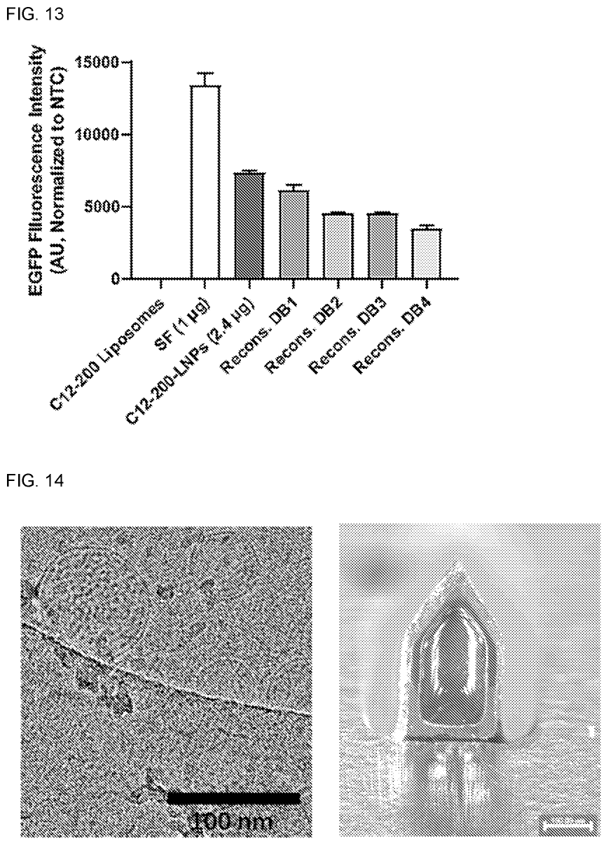

Twenty-four hours following the treatments, fluorescence images were acquired by the fluorescence microscope for the wells assigned for EGFP transfection. Cells were harvested and the cell pellets were re-suspended in 250 μL (6-well plate) and 150 μL (24-well plate) of the Cell Lysis Buffer. Cells were incubated for 10 minutes on ice, then spun at 12,000×g for 10 min. Lysates were diluted and evaluated for expression of EGFP proteins by Qubit 3 Fluorometer (FIG. 12) and HA proteins by the ELISA immunoassay against the rHA protein standard plot (FIG. 13), respectively.

Acquired images clearly demonstrated bright signals of EGFP in the cells treated with the positive control SF (StemFect) and C12-200-LNPs (FIG. 12). No detectable fluorescence signals were observed in the cells treated with vacant liposomes or those with no treatment (NTC). Using vacant liposomes made from C12-200, it was confirmed that the fluorescence detected signals are from the expressed GFP proteins. EGFP signals were also observed for the cells treated with C12-200 LNPs reconstituted in DB1, 2, 3, and 4.

Quantified fluorescence intensity revealed that C12-200-LNPS achieved a high level of expression of ˜55% of that of SF and all reconstituted C12-200 LNPs retained 50-80% of the fresh LNPs (FIG. 13).

### Example 3: mRNA Vaccines for VaxiPatch

mRNA vaccines may prove a very good fit for the VaxiPatch platform. Unlike current mRNA vaccines, the VaxiPatch versions would be temperature-stable, raising the potential to eliminate the cold chain and revolutionize access and delivery of mRNA vaccines globally.

Examples of mRNA useful for vaccine used on VaxiPatch include, e.g., self-amplifying mRNA amplicons such as those disclosed in U.S. Pat. No. 10,022,436, which is incorporated by reference herein to its entirety. It has been shown that these examples of self-amplifying mRNA amplicon vaccines on VaxiPatch have a stability at room temperature.

FIG. 14 shows a CryoEM image of dried and reconstituted samRNA LNPs (the left panel). The data shows that high quality mRNA LNPs were prepared by using a NanoAssemblr. The mRNA LNPs were formulated into sugar glasses (FIG. 14, the right panel).

Examples of samRNAs were encapsulated into LNPs as shown in Table 2 below. The data shows high quality mRNA LNPs were made using a NanoAssemblr.

TABLE 2

| Free/membrane- | Total | Total RNA (x | Encapsulation |
|---|---|---|---|
| associated RNA | RNA | dilution factor) | Efficiency |
| ng/μl | ng/μl | ng/μl | % |
| 5.48 | 63.20 | 0.51 | 91 |
| 3.64 | 61.46 | 0.49 | 94 |
| 4.96 | 61.86 | 0.49 | 92 |

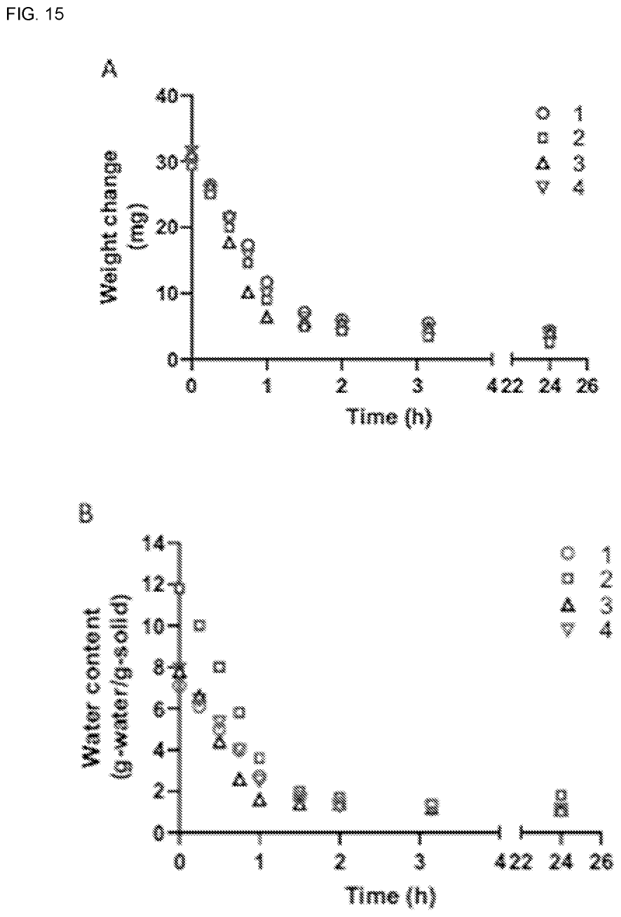

A sugar glass is truly a unique form of matter that has a very high water content, is a solid and confers room temperature stability. Sugar glass water content can be important for stability. FIG. 15 shows a measurement of water contents in an example of sugar glass, showing the loss of water contents in sugar glass through time. To measure the water content of LNPs over time, LNPs were diluted 9-fold in 15% tre/PBS to an RNA concentration of 0.5-0.6 mg/mL of RNA. LNPs (15 μL) was added to 15 μL of 15% tre/PBS in a pre-weighed piece of aluminum foil ˜1 cm×1 cm (7 mg). The piece of aluminum foil was placed in the cap of 1.5-mL Eppendorf tubes and dried under vacuum at room temperature. At indicated timepoints (0-24 h), samples were removed, and the aluminum foil squares were weighed. As control, 30 μL of 15% trehalose was added to a pre-weighed 0.5-mL Eppendorf tube and dried under vacuum for 24 h. Samples were then dried at 90° C. for 24 h, cooled to room temperature in a desiccator, and weighed to quantify the dry solid and calculate the water content. Four LNP samples displayed similar patterns of the water loss over time with water contents of about 2.6, 1.7 and 1.3 g·water/g·dried solid measured at 1 h, 1.5 h, and 24 h, respectively (Table 3). The control 15% trehalose weighed 4.55 mg after 24 h drying at 90° C., which is close to 4.5 mg, the calculated weight of 30 μL of 15% trehalose.

Activity of mRNA LNPs as measured on 293T cells was inversely related to the water content. Compared to lyophilized cake material (1-5% water) the moisture contents of mRNA LNPs sugar glass material is very high (50-75% water).

TABLE 3

| | Water Content (g · |
|---|---|
| Time (hour) | water/g · solid) |
| 1.0 | 2.8 ± 0.8 |
| 1.5 | 1.7 ± 0.2 |
| 24.0 | 1.3 ± 0.4 |

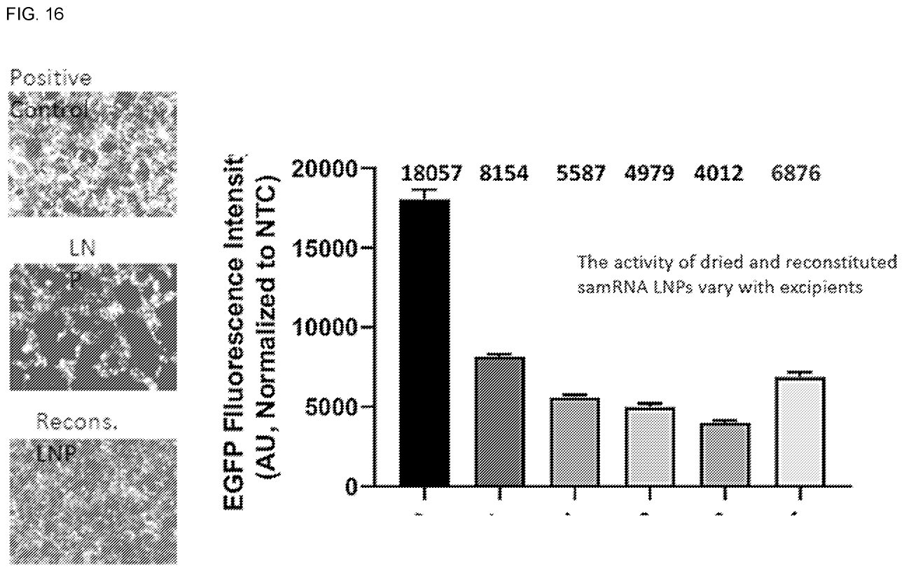

Examples of dried and reconstituted EGFP samRNA LNPs were found active. As shown in FIG. 16, after drying and reconstitution, there was 84% recovered activity remaining, compared to internal control, and 38% recovered activity remaining, compared to external control.

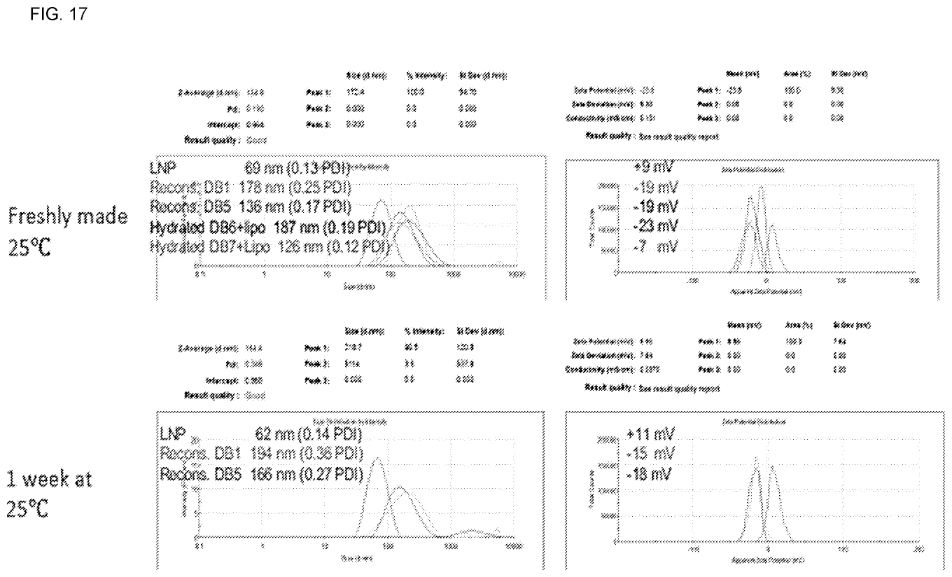

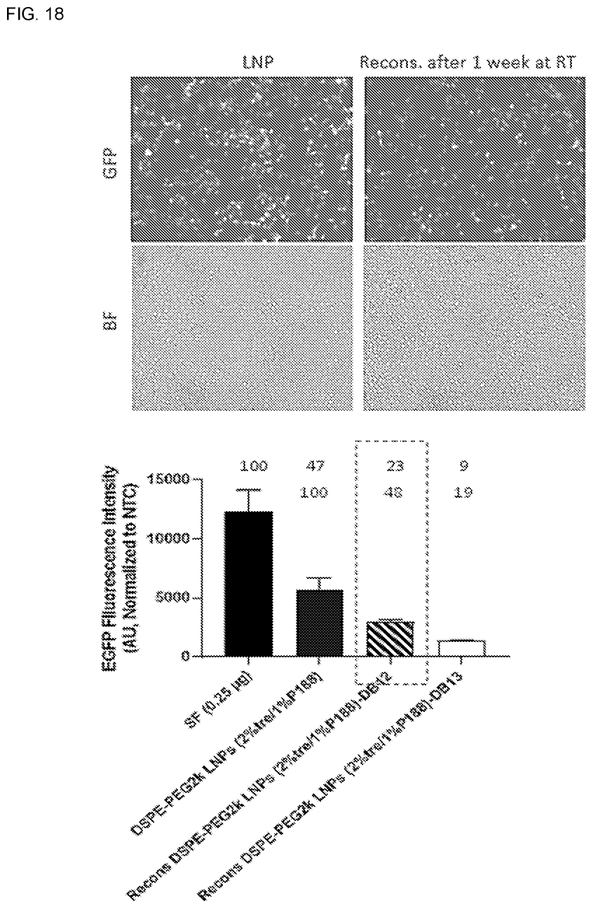

The stability at room temperature of the examples of samRNA-containing LNPs were measured and confirmed. As shown in FIGS. 17 and 18, after one week storage at 25° C., the LNPs still had comparable activity. Experiments in FIG. 18 were performed using C12-200-EGFP-samRNA-LNPs, made with DSPE-PEG2k (or PEG 2000) in the presence of 2% trehalose/1% P188. Specifically, LNPs were concentrated in 15% trehalose in PBS. Two LNP samples were dried with the following formulations for 1 week at RT under vacuum and rehydrated with 30 μl of the rehydrating buffers that included DOPC: Chol liposomes. Liposomes were made in 15% trehalose in PBS. Reconstitution steps for LNPs (the right-side two columns in FIG. 18) include:
1. Drying: 15 μL LNP in 15% trehalose in PBS+15 μL 15% trehalose in PBS (15% trehalose), using DB12 (15% trehalose+100 μg DOPC/cholesterol liposomes); Rehydration: 10 μL DOPC: Chol liposomes in 15% trehalose in PBS+20 μL 15% trehalose in PBS; or
2. Drying: 15 μL LNP in 15% trehalose in PBS+7.5 μL PBS+7.5 μL of 20% P188 in PBS (7.5% trehalose/5% P188), using DB13 (15% trehalose+1.7% P188+100 μg DOPC/cholesterol liposomes);
Rehydration: 10 μL DOPC: Chol liposomes in 15% trehalose in PBS+15 μL PBS +5 μL 20% P188.

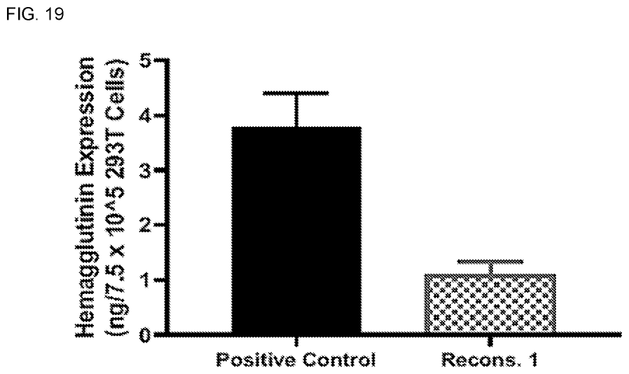

FIG. 19 shows that dried and reconstituted influenza hemagglutinin (HA) samRNA LNPs were still active.

### Example 4: Further Improving Stability of Examples of mRNA Vaccines after Storage at Room Temperature

Appropriately formulated mRNA LNPs after drying, storage and reconstitution (DS&R) does not cause LNP fusion, and thereby retains mRNA function. For example, examples of dried and reconstituted samRNA LNPs were found active at least for 1 week under room-temperature. The stability was shown below by comparing Dynamic Light Scattering (DLS) and Zeta potential for the samRNA LNP formulations freshly made versus being dried using drying buffer DB1 or DB2 as in Example 2, stored at 25° C. for one week, and then reconstituted. They have similar sizes, PDI readings, and zeta potentials.

### Freshly Made Formulations (Dried, Stored at 25° C. for 24 h, and Reconstituted)

| LNP formulations | Size (nm) and PDI | Zeta Potential (mV) |
|---|---|---|
| LNP (dried mRNA LNP) | 69 and 0.13 | +9 |
| DS&R DB1 (DB1) | 178 and 0.25 | −19 |
| DS&R DB2 (DB2) | 138 and 0.17 | −19 |

### Formulations (Dried, Stored at 25° C. for One Week, and Reconstituted)

| LNP formulations | Size (nm) and PDI | Zeta Potential (mV) |
|---|---|---|
| LNP (dried mRNA LNP) | 62 and 0.14 | +11 |
| DS&R DB1 (DB1) | 194 and 0.36 | −15 |
| DS&R DB2 (DB2) | 166 and 0.27 | −118 |

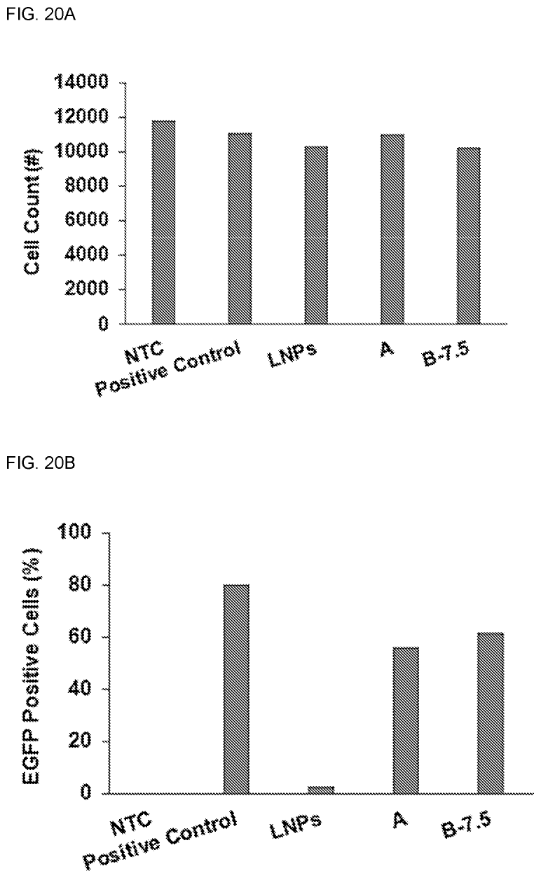

To further improve stability of mRNA LNP, more examples of LNP formulations were prepared to include, e.g., the ionizable lipid C12-200, DOPC, cholesterol (31:11:44), 0.1% DSPE-PEG2K and EGFP samRNAs, plus liposomes (FIGS. 20A, 20B, 21 and 22). Formulations were compared for their activity and toxicity to cells after drying, storage and reconstitution (DS&R). As shown in FIGS. 20A and 20B, parental EGFP samRNA LNPs were no longer toxic to cells but were inactive without DS&R, while DS&R EGFP samRNA LNPs were active in 80% of 293T cells as compared to the Stemfect® positive control.

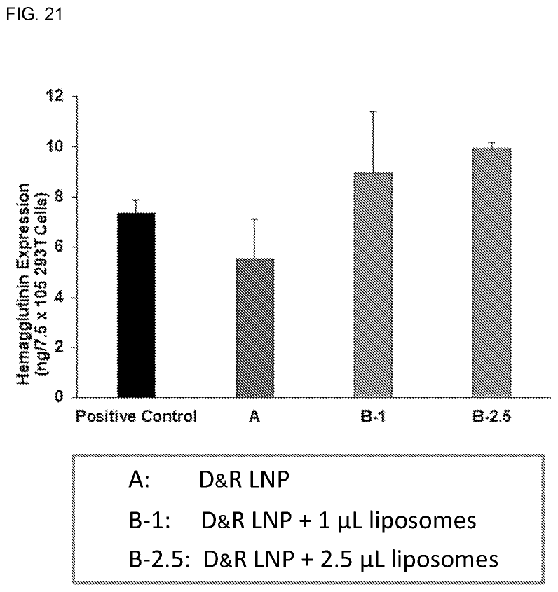

Different excipient conditions also affect LNP activity. For example, in FIG. 21,

DS&R samRNA Influenza Hemagglutinin (HA) LNPs made with an excipient were more active in the protein expression of HA in 293T cells than the Stemfect® samRNA HA LNP positive control. FIG. 21 shows LNPs prepared with 0.1% DSPE-PEG2K with liposomes as an excipient following DS&R retain 100% of the mRNA activity of the Stemfect® control. Repeating this experiment with varying DSPE-PEG2k from 0.1% to 2% show an inverse HA expression activity relationship between liquid LNPs and LNPs following DS&R.

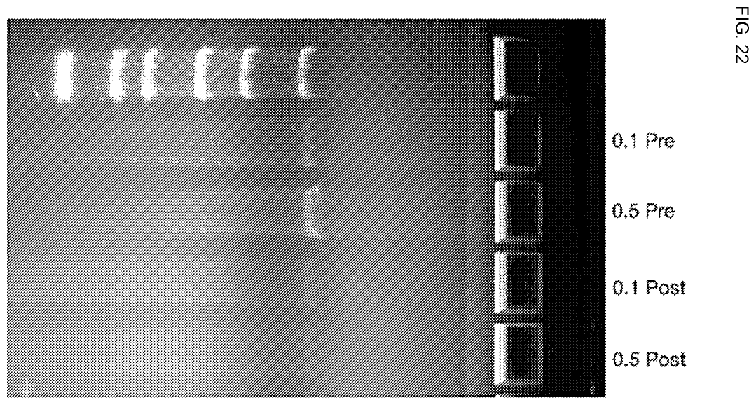

In an example of microarray, dried HA samRNA was very stable on VaxiPatch. As shown in FIG. 22, HA samRNA encapsulated in LNPs, printed on VaxiPatch, was dried and stored at room temperature (25° C.) for one week, and then reconstituted, extracted and analyzed by gel electrophoresis. The HA samRNA was physically intact after storage at 25° C. for one week. Similar experiment also showed that HA samRNA was physically intact after storage at 25° C. for four weeks.

### Example 5: Formulations for Printing on VaxiPatch

Further studies were carried out on components and the ratio between components for compositions and/or formulations described herein, for a better effect to increase their stability after drying, storage, and reconstitution (DS&R), especially involving storage at a temperature higher than the traditional −80° C. (e.g., at room temperature) for nucleic acids (e.g., RNAs) for a time period.

One example of formulation for the LNPs contains a molar ratio of components as follows:
51.2% cholesterol (structural lipid 1)
36.5% C12-200 (ionizable lipid)
11.8% DSPC (structural lipid 2) and
0.5% DSPE-PEG2000 (PEG coating).

Different PEG varieties were tested, such as DMG-PEG2000 (having a shorter hydrophobic anchoring tail) and DSPE-PEG2000 (having a longer anchoring tail). DMG-PEG2000 were found to have a cellular toxicity in the 293T experiments described herein (e.g., in Example 2), when used at, e.g., a 2% molar ratio. More PEG mole ratios between 0 and 2% were tested. Changes in such percentage of PEG come out of the cholesterol portion and, thus, do not affect ionizable lipid or DSPC percentages by mole fraction. As cholesterol has a low MW and PEG2000 has a high one, this changes mass ratios more substantially. In some embodiments, the compositions and formulations described herein contain about 0.01%, 0.05%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.5%, 4%, 4.5%, 5%, 5.5%, 6%, 6.5%, 7%, 7.5%, 8%, 8.5%, 9%, 9.5%, 10%, or more (and all sub-values and sub-ranges there between, including endpoints) PEG molecules. In some embodiments, the compositions and formulations described herein contain a PEG molecule in a percentage with minimal cellular toxicity. For example, in some embodiments, the compositions and formulations described herein contain about 0.1% to about 0.5% PEG molecules.

To assemble the LNPs, the lipid mix (in ethanol) described above in this Example was mixed with RNA in an acetate buffer at a controlled ratio to form the particles. The total lipid-to-mRNA ratio by mass was 1:28.74 (i.e., 174 μg of samRNA per 5 mg of total lipid). For use of the liposomal excipient, DOPC: cholesterol liposomes (at 33% molar ratio cholesterol, same as for VAS1.1) were used. A range of mass ratio of liposomes to total LNP lipids were tested, including the currently preferred range of 0.05-0.125. These line up as 1 μg liposomes for every 8-20 μg of LNPs.

An example of the compositions and formulations used in the print mix contains a final 15% (w/v) of trehalose, irrespective of the concentration of the other components. As an example for a print mix slated for 20 nL prints at 1.5 μg mRNA per VaxiPatch, the following formulation was used:
2.083 mg/mL samRNA (formulated in LNPs)
59.875 mg/mL LNPs (total LNP lipid from ratios above)
3 mg/mL DOPC/chol liposome excipient (assuming 0.05 mass ratio above)
150 mg/mL trehalose (15% w/v final)
9 mg/mL NaCl (from the PBS/formulation buffer) and
5 mg/mL FD&C blue No. 1 dye

The formulation solution was PBS (phosphate buffer 6.7 mM and salt at 137 mM/0.9% w/v) in the print mix, containing a small amount of phosphate and KCl as well.

As already noted, vaccine formulations used with VaxiPatch have been shown to maintain stability at room temperature for at least 28 days, maintaining substantial activity even after rehydration.

### Example 6: Compositions Containing Other RNA Molecules

Compositions and formulations described herein may be used to deliver different types of nucleic acids into a cell or a subject. For example, besides the examples of mRNA and samRNAs vaccines described herein, siRNA or other RNA molecules may be encapsulated into a LNP in the compositions and formulations described herein. An example of using siRNA-LNP formulations may be found in Kauffman et al., Nano Lett. 2015; 15:7300-7306, the contents of which is incorporated by reference herein to its entirety.

### Example 7: Preparation of mRNA LNPs Using SM-102

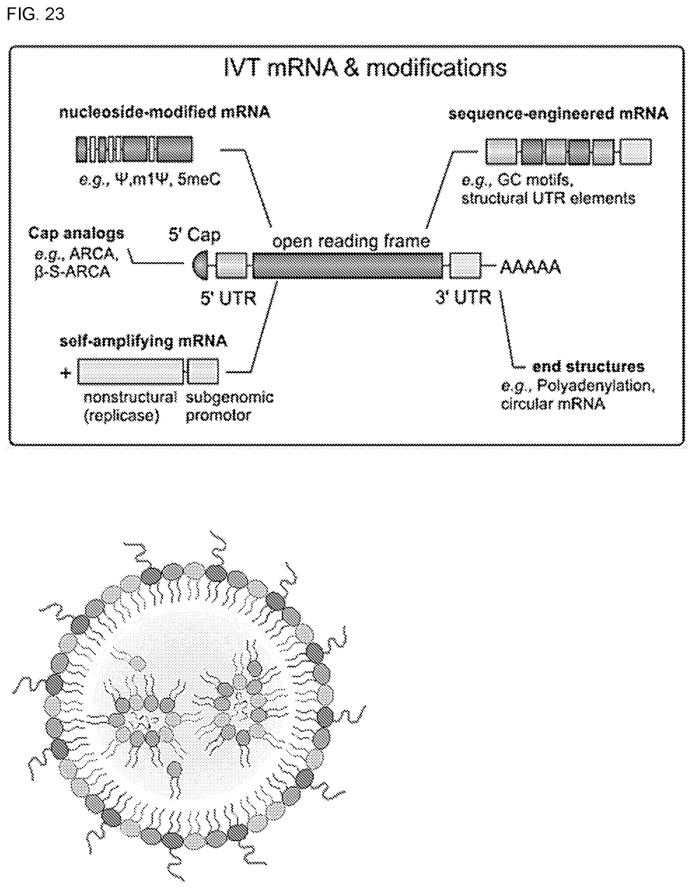

mRNA-containing LNPs were made utilizing formulation ingredients previously approved by FDA for existing COVID vaccines. An example RNA construction amenable for use in LNP production is shown in FIG. 23.

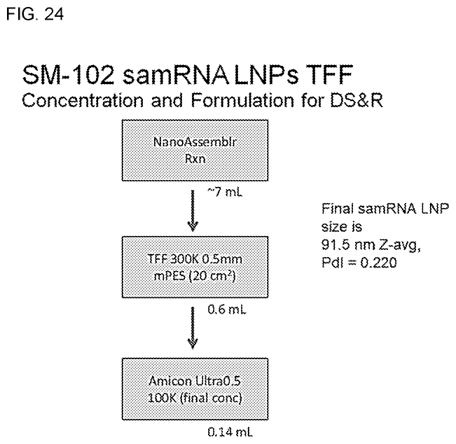

An example of a process used to make self assembling mRNA LNPs is shown in FIG. 24. In one example, samRNAs-containing LNPs were produced with ionizable lipid, SM-102. The LNP preparation was similar to that described above in conection with C12-200, but with SM-102 instead. The LNPs were prepared using the same ingredients as the existing Moderna COVID vaccine, but with a different RNA. The LNPs included structural lipids (e.g., cholesterol and DSPC), DMG-PEG2000, SM-102 (50:1.5:10:38.5 mol %) and other excipients. The particular RNA used in this example was HA mRNA. As shown in FIG. 24, LNPs were assembled using NanoAssemblr, in a solution of a total volume of about 7 mL (pH 4.0). Tangential Flow Filtration (TFF) was then used to concentrate the LNP solution to about 0.6 mL and adjust the pH with PBS to the physiological pH 7.4. Trehalose was added to the solution. Amicon Ultra-0.5 mL Centrifugal Filters were further used [300 kDa, 0.5 mm mPES (modified polyethersulfone)] to concentrate the LNP solution to about 0.14 mL. Final samRNA LNP size in the solution is about 91.5 nm (Z-avg; PdI=0.220). The final trehalose concentration was approximately 20%.

The prepared formulations were stable at room temperature and retained activity after drying, storage and reconstitution. In one set of experiments activity was retained for at least one week.

### Example 8: Preparation of mRNA LNPs Using ALC-0315

In another example, samRNAs-containing LNPs were produced with the ionizable lipids, ALC-0315 and ALC-0519, rather than C12-200 or SM-102 as described in previous Examples, and with cholesterol and DSPC (46.3:1.6:9.4:42.7 mol %). The RNA utilized was an EGFP-samRNA. The same approach as used in Example 7 above was utilized and room temperature stable mRNA LNPs were produced. The average size of the LNP particles in one experiment was about 73 nanometers.

### Example 9: Formulations in Multi-Dose Glass Vial

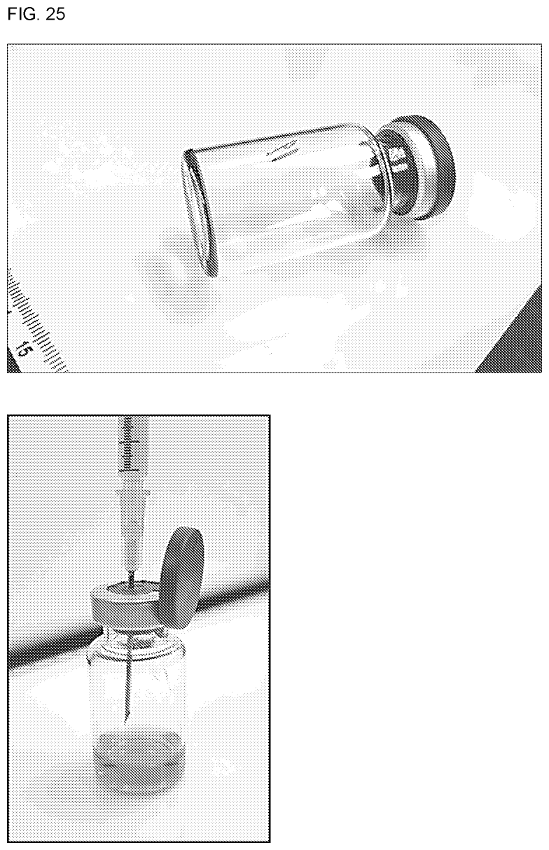

As noted herein, mRNA-containing LNP formulations described herein may be prepared, stored, transported, and/or reconstituted in kits or devices described herein, such as ampoules, vials, pre-filled syringes, etc. FIG. 25 shows an example of a dried samRNA-containing LNP formulation in the form of sugar glass at the bottom of a glass vial (with blue dye added to show the sugar glass material). The dried formulation included SM-102 as the ionizable lipid and also blue dye for illustration purposes. The top photo shows the dried formulation in the vial. The bottom photo shows reconstituted LNP formulation. The dried material was reconstituted in 2.5 mL PBS, which could then be used for up to five 0.5 mL doses.

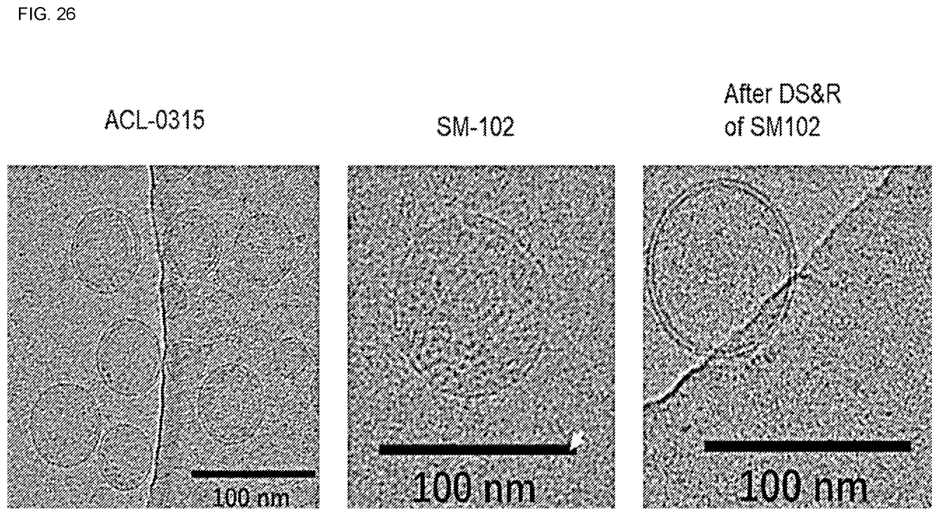

As shown in FIG. 26, LNPs utilizing different lipids (e.g., ALC-0315 and SM-102) were prepared and Cryo-EM images were obtained. The SM-102-containing LNPs prepared herein were additional reconstituted after the drying and storage (at room temperature) processes. Those LNPs after reconstitution maintained nanoparticle structure (FIG. 26) and maintained activity.

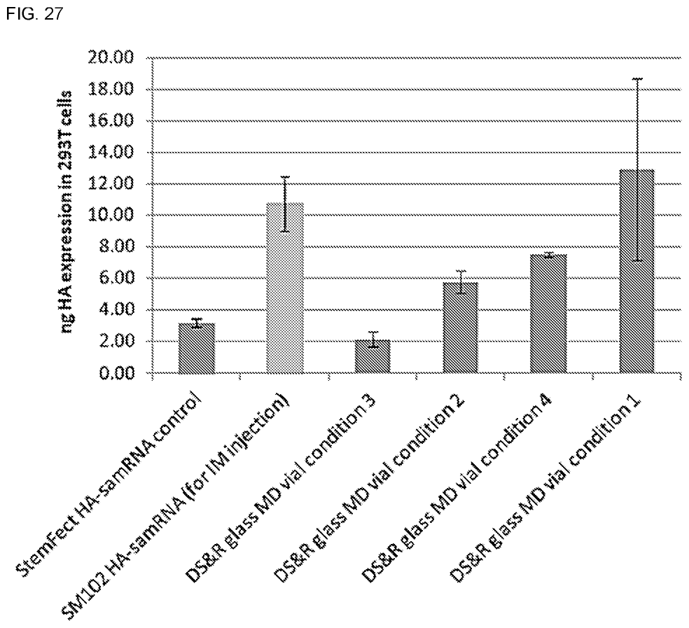

LNP formulations dried, stored and reconstituted (DS&R) in glass vials were prepared. As shown in FIG. 27, compared to the control conditions, the LNP formulation stored as sugar glass in glass vials maintained up to 100% mRNA activity (shown in HA expression levels in transfected 293T cells), under different conditions 1-4, after drying, storage at room temperature and reconstitution. SM-102 LNPs produced herein for DS&R contains 1.5% DMG-PEG and then mixed with VEE-HA samRNAs. Quantitative HA ELISA was performed at 24 h after transfection. Condition 1 showed the highest activity and had the highest final concentration of trehalose at about 20%. The other conditions 2-4 had lower amounts or concentrations of trehalose and showed activity, but lower than what was seen with 20% trehalose.

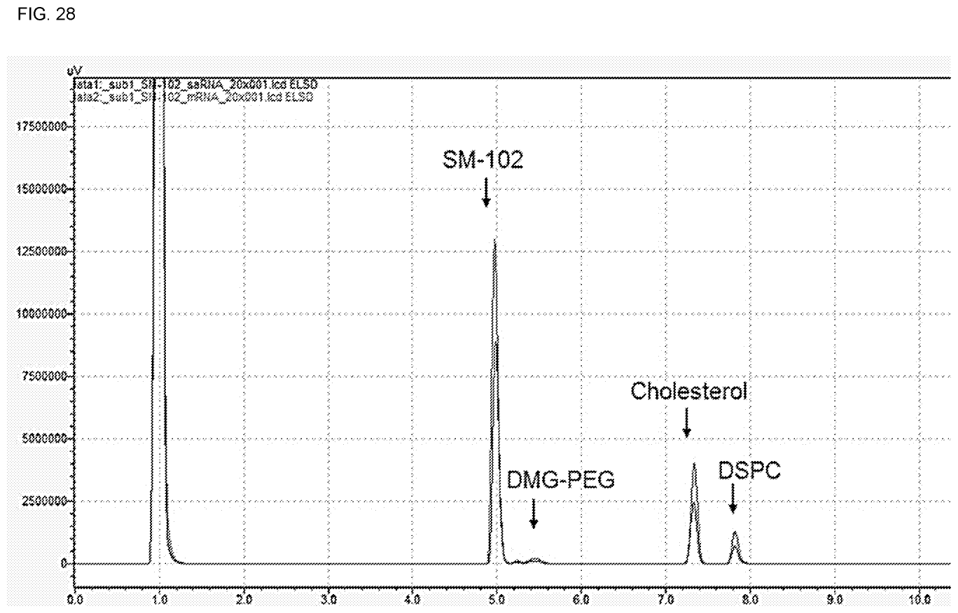

Analytical data confirmed that the LNP components for Moderna's COVID-19 mRNA vaccine and the LNP components described herein (for the HA-encoding samRNAs) are the same (FIG. 28).

### Example 10: Methods of Administering the LNP Formulations

Compositions and formulations described herein may be used to deliver different types of nucleic acids into a cell or a subject in various conditions. For example, the compositions and formulations may be administered as described herein (e.g., ampoules, vials, pre-filled syringes, etc.), or on any microarray devices (e.g., VaxiPatch). VaxiPatch:

The LNP/mRNA-based vaccines and therapeutics can be topically delivered. One approach is to use the VaxiPatch device. This device is a room-temperature-stable topical patch. The patch is easily applied to the skin. Light pressure is used to cause a “snap” mechanism to bring the materials on the patch into contact with the skin painlessly. After a certain time period (e.g., 5 minutes), the patch can be removed and discarded. In this non-limiting patch example, no syringes or trained professionals are required. In testing that has been done, little to no skin irritation has been evident within, e.g., 48 hours of application.

### Example 11: Improving Activity of mRNA LNP Formulations after DS&R

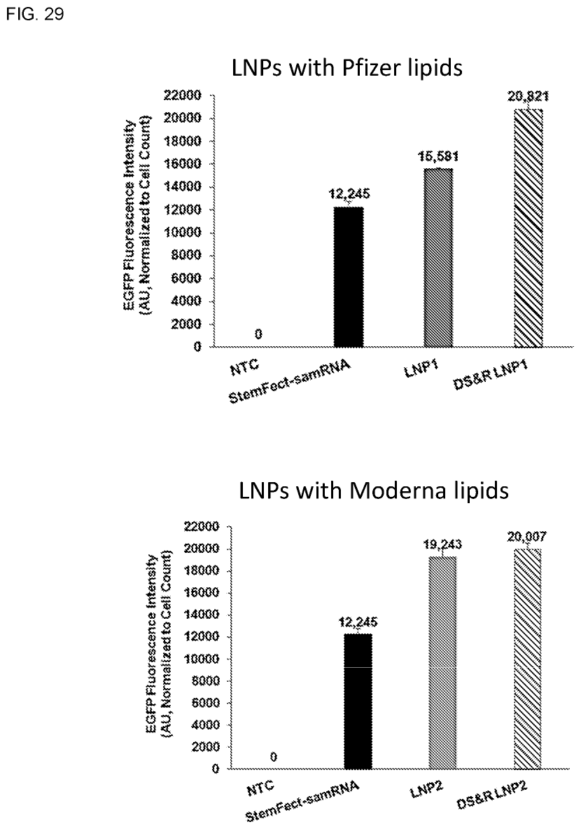

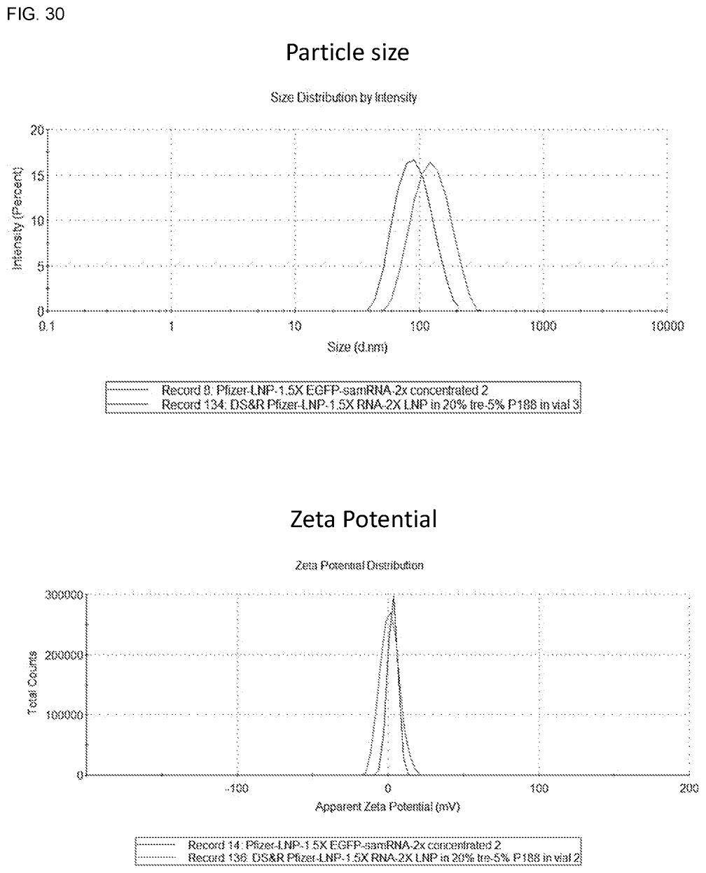

Exemplary samRNA LNPs were analyzed for their activity after drying, storage and reconstitution (DS&R). As described in the previous Examples, samRNA replicons expressing EGFP were prepared with the lipids used in the COVID-19 vaccine produced by Pfizer (e.g., ALC-0315) or Moderna (e.g., SM-102) were dried, stored, and then reconstituted for testing the RNA stability. As shown in FIG. 29, LNPs with either Pfizer (the top panel) or Moderna (the bottom panel) lipids recovered full activity after DS&R. DLS (Dynamic Light Scattering) shows that the exemplary Pfizer-LNP formulation following DS&R grew to 152 nm from 86 nm and remained neutral without significant change in the particle surface charge (FIG. 30 and the table below).

| | | | Zeta |
|---|---|---|---|
| | Size | | Potential |
| LNP Formulation | (nm) | PDI | (mV) |
| Pfizer-EGFP-samRNA-LNPs- | 86 | 0.14 | +3 |
| ALC-0315 | | | |
| After DS&R | 152 | 0.12 | +1 |
| Pfizer-EGFP-samRNA-LNPs - | | | |
| ALC-0315 | | | |

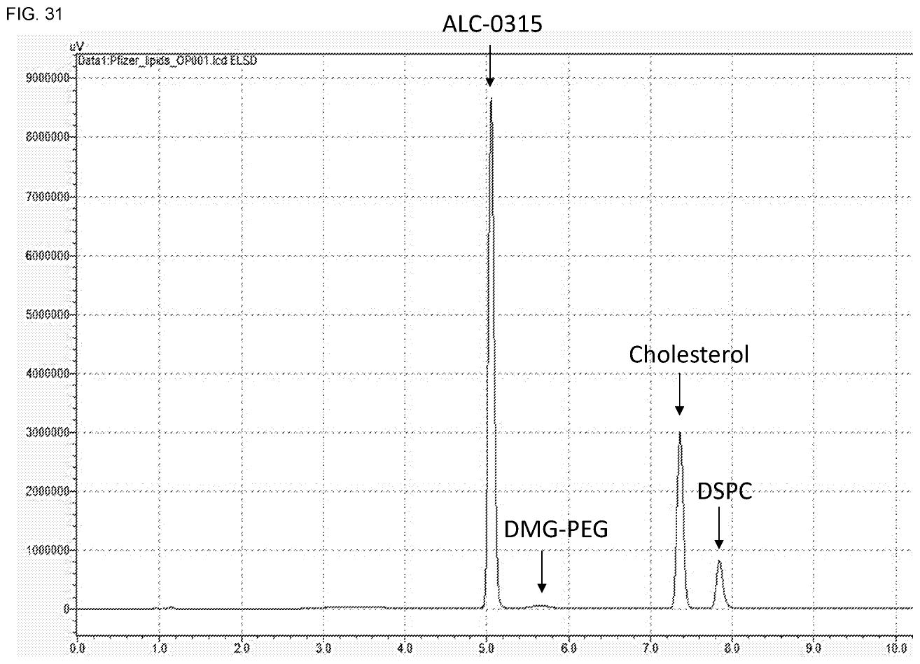

The existence of components in the Pfizer LNP formulations is shown by HPLC (FIG. 31).

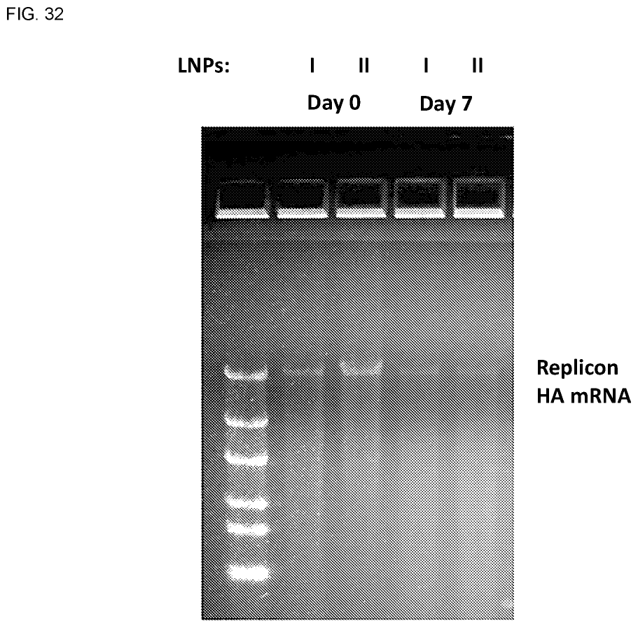

Similar experiments were performed with HA-expressing mRNA LNP formulations. HA mRNA encapsulated in LNPs were dried, stored at room temperature (25° C. for 7 days), and reconstituted, as described in the previous Examples. After reconstitution, mRNA were extracted and analyzed by gel electrophoresis, showing that the mRNA was physically intact after 7 days of storage at room temperature (FIG. 32). LNPs printed on VaxiPatch were further tested and found to be stable under the DS&R process after 28 days of storage at 25° C.

RNAse protection assay was used to test the stability of LNP formulation containing the Pfizer lipids. The main steps for such assay include: Add 3.8 mAU RNase per 1 μg RNA;
Incubate 30 minutes at RT;
Add Proteinase K to remove RNase;
Incubate for 15 minutes at 55 degrees Celsius;
Extract the RNA with an excess of Ethanol;
Centrifuge at 14,000×g for 10 minutes;
Remove supernatant and dry with argon stream;
Resuspend RNA pellet;
Denature RNA in 2× formaldehyde loading buffer;
Incubate at 65 degrees Celsius for 10 minutes; and
Load 200 ng RNA per lane for gel electrophoresis.

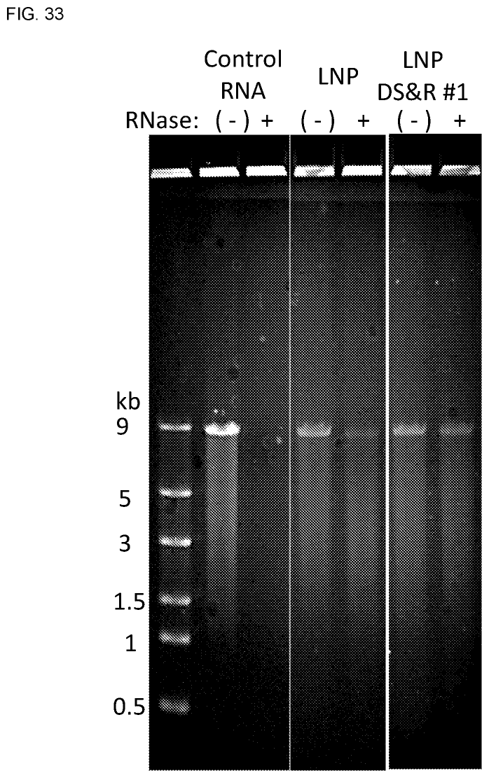

As shown in FIG. 33, the Pfizer LNP formulations were stable and protected after the DS&R process.

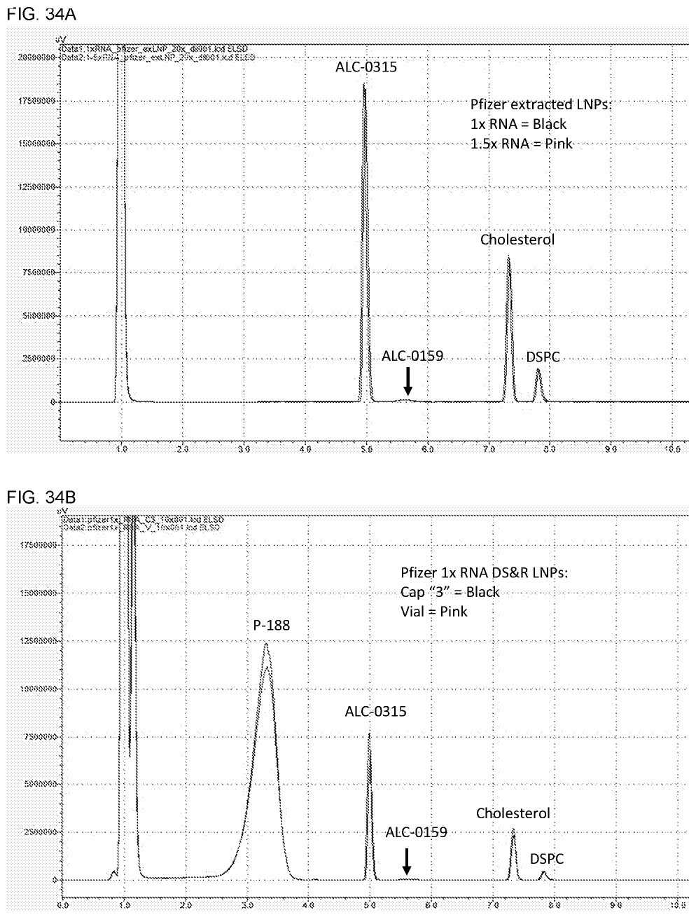

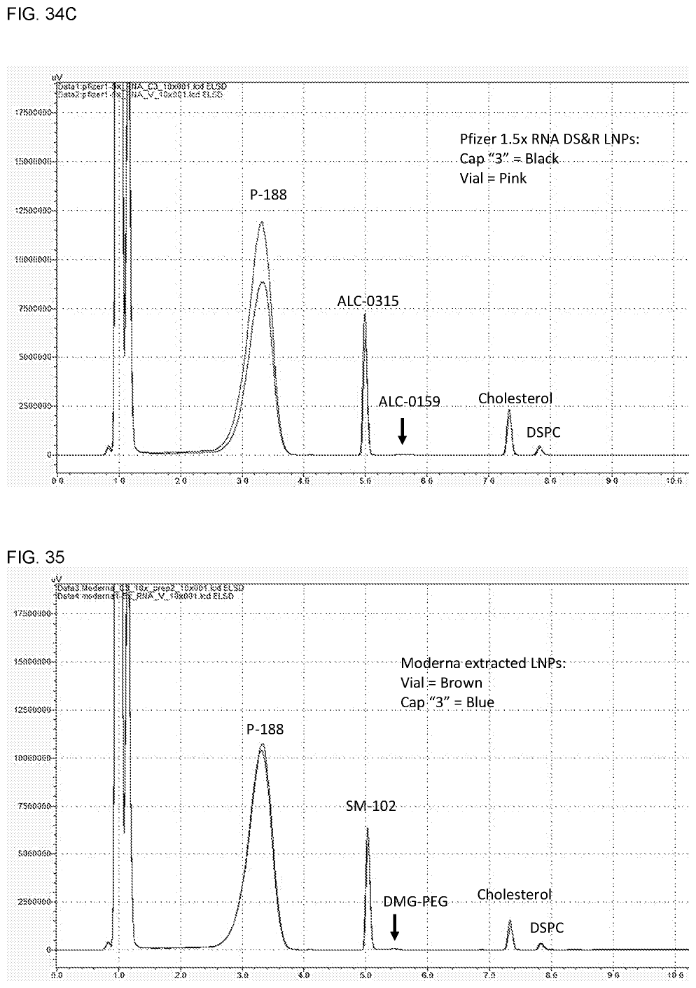

The existence of components in various Pfizer LNP formulations is shown by HPLC (FIGS. 34A-34C and the table below).

Pfizer LNPs HPLC analysis (μg/mL)

| | | | | | Total |
|---|---|---|---|---|---|
| | | | ALC- | ALC- | lipid |
| | Cholesterol | DSPC | 0159 | 0315 | concentration |
| Organic Phase | 1573.796 | 1123.915 | 369.975 | 4887.753 | 7955.439 |
| 1.5X RNA post | 434.283 | 297.482 | 118.582 | 1375.496 | 2225.845 |
| NanoAssemblr | | | | | |
| 1X RNA post | 424.803 | 298.508 | 81.444 | 1364.642 | 2169.397 |
| NanoAssemblr | | | | | |
| 1.5X RNA pfizer | 4849.220 | 2552.994 | 1093.220 | 13193.308 | 21688.742 |
| LNP | | | | | |
| 1X RNA pfizer | 4662.144 | 2556.859 | 1109.997 | 12964.065 | 21293.065 |
| LNP | | | | | |
| DS&R LNP Cap 3 | 806.875 | 573.922 | 200.859 | 2735.539 | 4317.195 |
| 1.5x RNA | | | | | |
| DS&R LNP Cap 3 | 806.585 | 563.068 | 189.850 | 2506.700 | 4066.203 |
| 1x RNA | | | | | |
| DS&R LNP Vial | 721.329 | 536.932 | 181.680 | 2295.358 | 3735.299 |
| 1.5x RNA | | | | | |
| DS&R LNP Vial | 911.584 | 579.472 | 214.342 | 2861.768 | 4567.166 |
| 1x RNA | | | | | |

Exemplary modern LNPs were prepared similarly in process stages, including Organic Phase (lipids in ethanol before NanoAssemlbr), Post NanoAssemblr (after the NanoAssemblr reaction, but before removal of ethanol by either TFF or Amicon), After Amicon buffer exchange and concentration, and After DS&R in a vial or under Cap 3 conditions. Both of vial and Cap 3 samples were dried in 20% trehalose and 5% P-188. The existence of components in these Moderna LNP formulations is shown by HPLC (FIG. 35 and the table below). The percentage of P-188 (w/v) is 3.51% in vial and 3.59% in Cap 3.

Moderna LNP preparation HPLC concentration (μg/mL)

| | | | | | | RNA |
|---|---|---|---|---|---|---|
| | Cholesterol | DSPC | DMG-PEG | SM-102 | Total lipid | concentration |
| Organic Phase | 1362.027 | 1156.855* | 724.726 | 4164.151 | 7407.759 | N/A |
| post NanoAssemblr | 442.680 | 335.423 | 224.275 | 1307.912 | 2310.290 | Not measured |
| Extracted LNP | 3526.250 | 2228.520 | 1487.571 | 10234.809 | 17477.150 | 820 |
| DS&R vial | 586.358 | 515.867* | 323.440 | 1833.328 | 3258.993 | 205 |
| DS&R Cap 3 | 508.370 | 521.980* | 330.210 | 1654.320 | 3014.880 | 205 |

*below lowest standard

While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

What is claimed is:

1. A composition comprising a RNA molecule and a lipid nanoparticle (LNP), wherein the RNA molecule is encapsulated in the LNP, wherein the composition is in the form of sugar glass.

2. The composition of claim 1, wherein the RNA molecule is a self-amplifying RNA (samRNA).

3. The composition of claim 1, wherein the RNA molecule is a messenger RNA (mRNA), a small interfering RNA (siRNA), a short hairpin RNAs or small hairpin RNA (shRNA), a microRNA (miRNA), a miRNA inhibitor (antagomirs/antimirs), or a messenger-RNA-interfering complementary RNA (micRNA).

4. The composition of any one of claims 1 to 3, wherein the RNA molecule encodes a viral polypeptide or a fragment thereof.

5. The composition of claim 4, wherein the viral polypeptide is or is derived from a spike protein of a virus.

6. The composition of claim 5, wherein the virus is a coronavirus or an influenza virus.

7. The composition of claim 6, wherein the coronavirus is SARS-COV-2.

8. The composition of any one of claims 4 to 7, wherein the RNA molecule is a vaccine against a virus comprising the viral polypeptide or fragment thereof to be administered to a subject in need of.

9. The composition of any one of claims 1 to 8, further comprising a second RNA molecule encapsulated in the LNP.

10. The composition of any one of claims 1 to 9, wherein the sugar glass comprises one or more of sucrose, glucose, galactose, fructose, trehalose, and maltose.

11. The composition of claim 10, wherein the sugar glass comprises trehalose.

12. The composition of any one of claims 1 to 11, further comprising a PEG molecule, an ionizable lipid, and/or a structural lipid.

13. The composition of any one of claims 1 to 12, wherein the LNP comprises an ionizable lipid.

14. The composition of claim 13, wherein the ionizable lipid comprises one or more of DLin-MC3-DMA, C12-200, ALC-0315, ALC-0519, A9, and SM-102.

15. The composition of any one of claims 12-14, wherein the PEG molecule comprises one or more of polyethylene glycol 2000 (PEG 2000), DMG-PEG2000 (polyethylene glycol 2000 dimyristoyl glycerol), ALC-0159 (2 [(polyethylene glycol)-2000]-N,N-ditetradecylacetamide) and DSPE-PEG2000.

16. The composition of claim 15, wherein the PEG molecule comprises DSPE-PEG2000.

17. The composition of any one of claims 1 to 16, wherein the structural lipid comprises one or more of cholesterol, 1,2-distearoyl-sn-glycero-3-phosphocholine (DSPC), 1,2-distearoyl-sn-glycero-3-phosphorylethanolamine (DSPE), and DSPE derivatives.

18. The composition of any one of claims 1 to 17, comprising a structural lipid, an ionizable lipid and a PEG molecule in a molar ratio of about 27%-about 40% ionizable lipid, about 45%-about 70% structural lipid and about 0.1%-about 3% PEG molecule.

19. The composition of claim 18, comprising a molar ratio of about 35%-about 37% ionizable lipid, about 62%-about 64% structural lipid and about 0.3%-about 1% PEG molecule.

20. The composition of claim 19, comprising a molar ratio of about 36.5% ionizable lipid, about 62% structural lipid and about 0.5% PEG molecule.

21. The composition of any one of claims 1 to 20, wherein sugar is present in the composition in amount of about 5%-about 20% (w/v).

22. The composition of claim 21, wherein sugar is present in the composition in amount of about 12%-about 17% (w/v).

23. The composition of claim 22, wherein the sugar is present in the composition in amount of about 15% (w/v).

24. The composition of any one of claims 1 to 23, wherein the water content in the sugar glass is about 0.1 to 10, 0.1 to 5, 0.1 to 3, 0.5 to 10, 0.5 to 5, 0.5 to 3, 1 to 10, 1 to 5, or 1 to 3 g·water/g·solid.

25. The composition of any one of claims 1 to 24, having an increased stability compared to a similar composition not in the form of sugar glass.

26. The composition of claim 25, wherein the increased stability is at least about 10%, 20%, 30%, 40%, 50%, 60%, 70%, 80%, 90%, 100%, 150%, 200%, 250%, 300%, 350%, 400%, 450%, 500%, 600%, 700%, 800%, 900%, 1000%, 2000%, 5000%, 10000%, or more.

27. The composition of claim 25 or 26, having an increased stability at a non-freezing temperature, or the room temperature.

28. The composition of any one of claims 1 to 27, capable of being reconstituted after a time period of storage at the non-freezing temperature or room temperature for administration to a subject in need of.

29. The composition of claim 28, wherein the time period of storage is about 1 day, 3 days, one week, 10 days, two weeks, three weeks, 28 days, one month, or longer.

30. The composition of any one of claims 1 to 29, further comprising an agent capable of facilitating the encapsulation.

31. The composition of claim 30, wherein the agent comprises P188 or HES.

32. The composition of claim 30 or 31, wherein the concentration of the agent in the composition is about 1%, 3%, 5%, 10%, 15%, 20%, 25%, 30%, 35%, 40%, or more.

33. The composition of any one of claims 1 to 32, wherein the composition has undergone vitrification.

34. A formulation of the composition of any one of claims 1 to 33, comprising a pharmaceutically acceptable excipient.

35. A vial or ampoule containing a sugar glass composition or formulation according to any one of claims 1-34.

36. A microneedle device for administering the composition of any one of claims 1 to 33 or the formulation of claim 34, comprising: (a) the composition of any one of claims 1 to 33 or the formulation of claim 34; and (b) a substrate comprising a sheet and a plurality of microneedles extending therefrom, each of said microneedles comprising a tip, a base, a hinge at the base connecting the microneedle to the sheet, and a well comprising the dehydrated composition.

37. A reconstituted composition for administration to a subject in need of, comprising the composition of any one of claims 1 to 33, or the formulation of claim 34.

38. The reconstituted composition of claim 37, wherein the reconstituted composition is in the vial or ampoule of claim 35.

39. A method of increasing stability of an RNA molecule in a composition, comprising formulating the RNA molecule with LNPs and forming the formulation into a sugar glass composition.

40. The method of claim 39, wherein the sugar glass composition comprising a composition according to any one of claims 1 to 33 or comprising the formulation of claim 34.

41. The method of claim 39 or 40, wherein the sugar glass is formed by vitrification.

42. The method of any of claims 39 to 41, wherein the increased stability is a stability at a non-freezing temperature or the room temperature.

43. A method of producing a composition of any one of claims 1 to 33 or the formulation of claim 34 for administering to a subject in need of, comprising
i) vitrifying the composition or formulation into sugar glass; and
ii) after a time period of storage at a non-freezing temperature or the room temperature, reconstituting the composition for administering.

44. A method of generating an immune response a subject in need thereof, comprising administering a therapeutically effective amount of the composition of any one of claims 1 to 33 or the formulation of claim 34 to the subject.

45. The method of claim 44, wherein the immune response is generated against an infectious agent.

46. The method of claim 45, wherein the infectious agent is a virus, a bacterium, a fungi or a protozoa.

47. A method of treating a virus-related disease or disorder in a subject in need of, comprising administering a therapeutically effective amount of the composition of any one of claims 1 to 33 or the formulation of claim 34 to the subject.

48. The method of any one of claims 44 to 47, wherein the composition or the formulation is reconstituted, after a time period of storage at a non-freezing temperature or the room temperature, prior to the administering.
